(19) United States
(12) Patent Application Publication
(10) Pub. No.: US 2008/0207616 A1
(43) Pub. Date: Aug. 28, 2008

(54) Quinoxalines as B Baf Inhhibitors

(75) Inventors: Brian Aquila, Marlborough, MA (US); Les Dakin, Natick, MA (US); Tracy Deegan, Salem, MA (US); Stephanos Ioannidis, Cambridge, MA (US); Stephen Lee, Waltham, MA (US); Paul Lyne, Arlington, MA (US); Timothy Pontz, Cambridge, MA (US); Mei Su, Marlborough, MA (US)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 11/577,132

(22) PCT Filed: Oct. 13, 2005
(86) PCT No.: PCT/GB05/03953

§ 371 Date: Apr. 12, 2007

Related U.S. Application Data
(60) Provisional application No. 60/619,373, filed on Oct. 15, 2004.

(51) Int. Cl. A61K 31/498; C07D 413/12; C07D 403/12; C07D 401/12; A61K 31/5377; A61P 35/00; C07D 407/12; C07D 409/12

(52) U.S. Cl. 514/234.8; 544/119; 544/353; 544/295; 514/249

(57) ABSTRACT

The invention relates to chemical compounds of the formula (I) or pharmaceutically acceptable salts thereof, which possess B Raf inhibitory activity and are accordingly useful for their anti cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm blooded animal such as man.

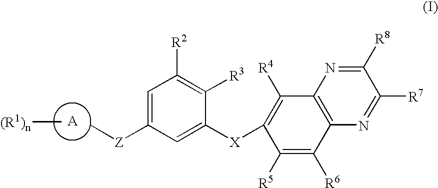

The invention relates to chemical compounds, or pharmaceutically acceptable salts thereof, which possess B-Raf inhibitory activity and are accordingly useful for their anti-cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm-blooded animal such as man.

The classical Ras, Raf, MAP protein kinase/extracellular signal-regulated kinase kinase (MEK), extracellular signal-regulated kinase (ERK) pathway plays a central role in the regulation of a variety of cellular functions dependent upon cellular context, including cellular proliferation, differentiation, survival, immortalization and angiogenesis (reviewed in Peyssonnaux and Eychene, Biology of the Cell, 2001, 93, 3-62). In this pathway, Raf family members are recruited to the plasma membrane upon binding to guanosine triphosphate (GTP) loaded Ras resulting in the phosphorylation and activation of Raf proteins. Activated Rafs then phosphorylate and activate MEKs, which in turn phosphorylate and activate ERKs. Upon activation, ERKs translocate from the cytoplasm to the nucleus resulting in the phosphorylation and regulation of activity of transcription factors such as Elk-1 and Myc.

The Ras/Raf/MEK/ERK pathway has been reported to contribute to the tumorigenic phenotype by inducing immortalisation, growth factor-independent growth, insensitivity to growth-inhibitory signals, ability to invade and metastasis, stimulating angiogenesis and inhibition of apoptosis (reviewed in Kolch et al., Exp. Rev. Mol. Med., 2002, 25 Apr., http://www.expertreviews.org/02004386h.htm). In fact, ERK phosphorylation is enhanced in approximately 30% of all human tumours (Hoshino et al., Oncogene, 1999, 18, 813-822). This may be a result of overexpression and/or mutation of key members of the pathway.

Three Raf serine/threonine protein kinase isoforms have been reported Raf-1/c-Raf, B-Raf and A-Raf (reviewed in Mercer and Pritchard, Biochim. Biophys. Acta, 2003, 1653, 25-40), the genes for which are thought to have arisen from gene duplication. All three Raf genes are expressed in most tissues with high-level expression of B-Raf in neuronal tissue and A-Raf in urogenital tissue. The highly homologous Raf family members have overlapping but distinct biochemical activities and biological functions (Hagemann and Rapp, Expt. Cell Res. 1999, 253, 34-46). Expression of all three Raf genes is required for normal murine development however both c-Raf and B-Raf are required to complete gestation. B-Raf −/− mice die at E12.5 due to vascular hemorrhaging caused by increased apoptosis of endothelial cells (Wojnowski et al., Nature Genet., 1997, 16, 293-297). B-Raf is reportedly the major isoform involved in cell proliferation and the primary target of oncogenic Ras. Activating somatic missense mutations have been identified exclusively for B-Raf, occurring with a frequency of 66% in malignant cutaneous melanomas (Davies et al., Nature, 2002, 417, 949-954) and also present in a wide range of human cancers, including but not limited to papillary thyroid tumours (Cohen et al., J. Natl. Cancer Inst., 2003, 95, 625-627), cholangiocarcinomas (Tannapfel et al., Gut, 2003, 52, 706-712), colon and ovarian cancers (Davies et al., Nature, 2002, 417, 949-954). The most frequent mutation in B-Raf (80%) is a glutamic acid for valine substitution at position 600. These mutations increase the basal kinase activity of B-Raf and are thought to uncouple Raf/MEK/ERK signalling from upstream proliferation drives including Ras and growth factor receptor activation resulting in constitutive activation of ERK. Mutated B-Raf proteins are transforming in NIH3T3 cells (Davies et al., Nature, 2002, 417, 949-954) and melanocytes (Wellbrock et al., Cancer Res., 2004, 64, 2338-2342) and have also been shown to be essential for melanoma cell viability and transformation (Hingorani et al., Cancer Res., 2003, 63, 5198-5202). As a key driver of the Raf/MEK/ERK signalling cascade, B-Raf represents a likely point of intervention in tumours dependent on this pathway.

AstraZeneca application WO 00/07991 discloses certain benzene-1,3-aminocarbonyl compounds which are inhibitors of the production of cytokines such as TNF, in particular of TNFα, and various interleukins, in particular IL-1. The present inventors have surprisingly found that certain benzene-1,3-aminocarbonyl compounds are potent B-Raf inhibitors and are accordingly expected to be useful in the treatment of neoplastic disease.

Accordingly, the present invention provides a compound of formula (I):

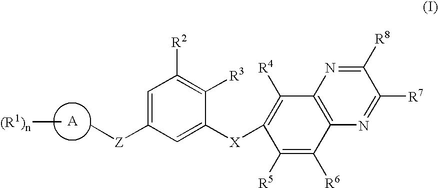

wherein:

Ring A is carbocyclyl or heterocyclyl; wherein if said heterocyclyl contains an —NH— moiety that nitrogen may be optionally substituted by a group selected from R⁹;

R¹ is a substituent on carbon and is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R¹⁰— or heterocyclyl-R¹¹—; wherein R¹ may be optionally substituted on carbon by one or more R¹²; and wherein if said heterocyclyl contains an —NH— moiety that nitrogen may be optionally substituted by a group selected from R¹³;

n is selected from 0-4; wherein the values of R¹ may be the same or different;

Z is —C(O)NH—, —NHC(O)— or —CH₂NH—;

R² is selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁴— or heterocyclyl-R¹⁵—; wherein R² may be optionally substituted on carbon by one or more R¹⁶; and wherein if said heterocyclyl contains an —NH— moiety that nitrogen may be optionally substituted by a group selected from R¹⁷;

R³ is selected from halo, hydroxy, methyl, methoxy or hydroxymethyl;

X is —NR¹⁸C(O)—, —NR¹⁹— or —NR²⁰CH₂—;

R⁴, R⁵, R⁶, R⁷ and R⁸ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R²¹— or heterocyclyl-R²²—; wherein R⁴, R⁵, R⁶, R⁷ and R⁸ independently of each other may be optionally substituted on carbon by one or more R²³; and wherein if said heterocyclyl contains an —NH— moiety that nitrogen may be optionally substituted by a group selected from R²⁴;

R¹⁸, R¹⁹ and R²⁰ are independently selected from hydrogen, C₁₋₆alkyl, C₁₋₆alkanoyl, C₁₋₆alkylsulphonyl, C₁₋₆alkoxycarbonyl, carbamoyl, N—(C₁₋₆alkyl)carbamoyl and N,N—(C₁₋₆alkyl)carbamoyl; wherein R¹⁸, R¹⁹ and R²⁰ independently of each other may be optionally substituted on carbon by one or more R²⁵;

R¹², R¹⁶, R²³ and R²⁵ are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R²⁶— or heterocyclyl-R²⁷—; wherein R¹², R¹⁶, R²³ and R²⁵ independently of each other may be optionally substituted on carbon by one or more R²⁸; and wherein if said heterocyclyl contains an —NH— moiety that nitrogen may be optionally substituted by a group selected from R²⁹;

R¹⁰, R¹¹, R¹⁴, R¹⁵, R²¹, R²², R²⁶ and R²⁷ are independently selected from a direct bond, —O—, —N(R³⁰)—, —C(O)—, —N(R³¹)C(O)—, —C(O)N(R³²)—, —S(O)ₛ—, —SO₂N(R³³)— or —N(R³⁴)SO₂—; wherein R³⁰, R³¹, R³², R³³ and R³⁴ is hydrogen or C₁₋₆alkyl and s is 0-2;

R⁹, R¹³, R¹⁷, R²⁴ and R²⁹ are independently selected from C₁₋₄alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R²⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;
with the proviso that said compound is not N-(5-{[3-(dimethylamino)benzoyl]amino}-2-methylphenyl)quinoxaline-6-carboxamide.

Accordingly, the present invention provides a compound of formula (I) which is a compound of formula (Ia):

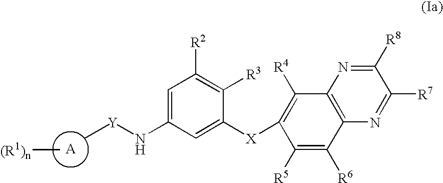

wherein:

Ring A is carbocyclyl or heterocyclyl; wherein if said heterocyclyl contains an —NH— moiety that nitrogen may be optionally substituted by a group selected from R⁹;

R¹ is a substituent on carbon and is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R¹⁰— or heterocyclyl-R¹¹—; wherein R¹ may be optionally substituted on carbon by one or more R¹²; and wherein if said heterocyclyl contains an —NH— moiety that nitrogen may be optionally substituted by a group selected from R¹³;

n is selected from 0-4; wherein the values of R¹ may be the same or different;

Y is —C(O)— or —CH₂—;

R² is selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₄alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁴— or heterocyclyl-R¹⁵—; wherein R² may be optionally substituted on carbon by one or more R¹⁶; and wherein if said heterocyclyl contains an —NH— moiety that nitrogen may be optionally substituted by a group selected from R¹⁷;

R³ is selected from halo, hydroxy, methyl, methoxy or hydroxymethyl;

X is —NR¹⁸C(O)—, —NR¹⁹— or —NR²⁰CH₂—;

R⁴, R⁵, R⁶, R⁷ and R⁸ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R²¹— or heterocyclyl-R²²—; wherein R⁴, R⁵, R⁶, R⁷ and R⁸ independently of each other may be optionally substituted on carbon by one or more R²³; and wherein if said heterocyclyl contains an —NH— moiety that nitrogen may be optionally substituted by a group selected from R²⁴;

R¹⁸, R¹⁹ and R²⁰ are independently selected from hydrogen, C₁₋₆alkyl, C₁₋₆alkanoyl, C₁₋₆alkylsulphonyl, C₁₋₆alkoxycarbonyl, carbamoyl, N—(C₁₋₆alkyl)carbamoyl and N,N—(C₁₋₆alkyl)carbamoyl; wherein R¹⁸, R¹⁹ and R²⁰ independently of each other may be optionally substituted on carbon by one or more R²⁵;

R¹², R¹⁶, R²³ and R²⁵ are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R²⁶— or heterocyclyl-R²⁷—; wherein R¹², R¹⁶, R²³ and R²⁵ independently of each other may be optionally substituted on carbon by one or more R²⁸; and wherein if said heterocyclyl contains an —NH— moiety that nitrogen may be optionally substituted by a group selected from R²⁹;

R¹⁰, R¹¹, R¹⁴, R¹⁵, R²¹, R²², R²⁶ and R²⁷ are independently selected from a direct bond, —O—, —N(R³⁰)—, —C(O)—, —N(R³¹)C(O)—, —C(O)N(R³²)—, —S(O)ₛ—, —SO₂N(R³³)— or —N(R³⁴)SO₂—; wherein R³⁰, R³¹, R³², R³³ and R³⁴ is hydrogen or C₁₋₆alkyl and s is 0-2;

R⁹, R¹³, R¹⁷, R²⁴ and R²⁹ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R²⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;
with the proviso that said compound is not N-(5-{[3-(dimethylamino)benzoyl]amino}-2-methylphenyl)quinoxaline-6-carboxamide.

Compounds of formula (Ia) are compounds of formula (I), therefore, unless otherwise stated all aspects of this invention that refer to compounds of formula (I) also refer to compounds of formula (Ia).

In this specification the term “alkyl” includes both straight and branched chain alkyl groups. References to individual alkyl groups such as “propyl” are specific for the straight chain version only and references to individual branched chain alkyl groups such as ‘isopropyl’ are specific for the branched chain version only. For example, “C₁₋₆alkyl” includes C₁₋₄alkyl, C₁₋₃alkyl, propyl, isopropyl and t-butyl. A similar convention applies to other radicals, for example “phenylC₁₋₆alkyl” includes phenylC₁₋₄alkyl, benzyl, 1-phenylethyl and 2-phenylethyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)—, and a ring sulphur atom may be optionally oxidised to form the S-oxides. Examples and suitable values of the term “heterocyclyl” are morpholino, piperidyl, pyridyl, pyranyl, pyrrolyl, pyrazolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. A particular example of the term “heterocyclyl” is pyrazolyl. In one aspect of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen-linked, a —CH₂— group can optionally be replaced by a —C(O)— and a ring sulphur atom may be optionally oxidised to form the S-oxides.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. A particular example of “carbocyclyl” is phenyl.

An example of “C₁₋₆alkanoyloxy” is acetoxy. Examples of “C₁₋₆alkoxycarbonyl” include methoxycarbonyl, ethoxycarbonyl, n- and 1-butoxycarbonyl. Examples of “C₁₋₆alkoxy” include methoxy, ethoxy and propoxy. Examples of “C₁₋₆alkanoylamino” include formamido, acetamido and propionylamino. Examples of “CC₁₋₆alkylS(O)ₐ wherein a is 0 to 2” include methylthio, ethylthio, methylsulphinyl, ethylsulphinyl, mesyl and ethylsulphonyl. Examples of “C₁₋₆alkanoyl” include propionyl and acetyl. Examples of “N—(C₁₋₆alkyl)amino” include methylamino and ethylamino. Examples of “N,N—(C₁₋₆alkyl)₂amino” include di-N-methylamino, di-(N-ethyl)amino and N-ethyl-N-methylamino. Examples of “C₂₋₆alkenyl” are vinyl, allyl and 1-propenyl. Examples of “C₂₋₆alkynyl” are ethynyl, 1-propynyl and 2-propynyl. Examples of “N—(C₁₋₆alkyl)sulphamoyl” are N-(methyl)sulphamoyl and N-(ethyl)sulphamoyl. Examples of “N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are N—(C₁₋₄alkyl)carbamoyl, methylaminocarbonyl and ethylaminocarbonyl. Examples of “N,N—(C₁₋₆alkyl)₂carbamoyl” are N,N—(C₁₋₄alkyl)₂carbamoyl, dimethylaminocarbonyl and methylethylaminocarbonyl. Examples of “C₁₋₆alkylsulphonyl” are mesyl, ethylsulphonyl and isopropylsulphonyl. Examples of “C₁₋₆alkylsulphonylamino” are mesylamino, ethylsulphonylamino and isopropylsulphonylamino. Examples of “C₁₋₆alkoxycarbonylamino” are methoxycarbonylamino and t-butoxycarbonylamino.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (I) may have chiral centres and/or geometric isomeric centres (E- and Z-isomers), and it is to be understood that the invention encompasses all such optical, diastereoisomers and geometric isomers that possess B-Raf inhibitory activity. The invention further relates to any and all tautomeric forms of the compounds of the formula (I) that possess B-Raf inhibitory activity.

It is also to be understood that certain compounds of the formula (I) can exist in solvated as well as unsolvated forms such as, for example, hydrated forms. It is to be understood that the invention encompasses all such solvated forms which possess B-Raf inhibitory activity.

Particular values of variable groups are as follows. Such values may be used where appropriate with any of the definitions, claims or embodiments defined hereinbefore or hereinafter.

It is to be understood that hereinbelow where particular values are described, with the exception of Y and Z which refer to formula (Ia) and (I) respectively, these particular values refer to both compounds of formula (I) and formula (Ia).

Ring A is carbocyclyl.

Ring A is heterocyclyl; wherein if said heterocyclyl contains an —NH— moiety that nitrogen may be optionally substituted by a group selected from R⁹.

Ring A is carbocyclyl or heterocyclyl; wherein if said heterocyclyl contains an —NH— moiety that nitrogen may be optionally substituted by a group selected from R⁹; wherein R⁹ is selected from C₁₋₆alkyl.

Ring A is carbocyclyl or heterocyclyl; wherein if said heterocyclyl contains an —NH— moiety that nitrogen may be optionally substituted by a group selected from R⁹; wherein R⁹ is selected from methyl or t-butyl.

Ring A is phenyl, pyrazolyl, benzimidazolyl, pyridyl, thienyl, furyl, 2,3-dihydro-1,4-benzodioxinyl, 2,3-dihydro-1-benzofuranyl, pyrimidinyl, imidazolyl, indolyl, pyrrolyl or pyrazinyl; wherein said pyrrolyl, pyrazolyl or imidazolyl may be optionally substituted on nitrogen by a group selected from R⁹; wherein R⁹ is selected from C₁₋₆alkyl.

Ring A is phenyl, pyrazolyl, benzimidazolyl, pyridyl, thienyl, 2,3-dihydro-1,4-benzodioxinyl, 2,3-dihydro-1-benzofuranyl, pyrimidinyl, imidazolyl, indolyl, pyrrolyl or pyrazinyl; wherein said pyrrolyl, pyrazolyl or imidazolyl may be optionally substituted on nitrogen by a group selected from R⁹; wherein R⁹ is selected from C₁₋₆alkyl.

Ring A is phenyl, pyrazol-3-yl, pyrazol-5-yl, benzimidazol-2-yl, pyrid-2-yl, pyrid-3-yl, pyrid-4-yl, thien-2-yl, thien-3-yl, fur-2-yl, 2,3-dihydro-1,4-benzodioxin-5-yl, 2,3-dihydro-1-benzofuran-7-yl, pyrimidin-4-yl, pyrimidin-5-yl, imidazol-2-yl, indol-4-yl, indol-7-yl, pyrrol-2-yl or pyrazin-2-yl; wherein said pyrrol-2-yl, pyrazol-3-yl, pyrazol-5-yl or imidazol-2-yl may be optionally substituted on nitrogen by a group selected from R⁹; wherein R⁹ is selected from methyl or t-butyl.

Ring A is phenyl, pyrazol-5-yl, benzimidazol-2-yl, pyrid-2-yl, pyrid-3-yl, thien-2-yl, 2,3-dihydro-1,4-benzodioxin-5-yl, 2,3-dihydro-1-benzofuran-7-yl, pyrimidin-5-yl, imidazol-2-yl, indol-4-yl, indol-7-yl, pyrrol-2-yl or pyrazin-2-yl; wherein said pyrrol-2-yl, pyrazol-5-yl or imidazol-2-yl may be optionally substituted on nitrogen by a group selected from R⁹; wherein R⁹ is selected from methyl or t-butyl.

Ring A is phenyl, 1-methylpyrazol-3-yl, 1-methylpyrazol-5-yl, 1-t-butylpyrazol-5-yl, benzimidazol-2-yl, pyrid-2-yl, pyrid-3-yl, pyrid-4-yl, thien-2-yl, thien-3-yl, fur-2-yl, 2,3-dihydro-1,4-benzodioxin-5-yl, 2,3-dihydro-1-benzofuran-7-yl, pyrimidin-4-yl, pyrimidin-5-yl, 1-methylimidazol-2-yl, indol-4-yl, indol-7-yl, 1-methylpyrrol-2-yl or pyrazin-2-yl.

Ring A is phenyl, 1-t-butylpyrazol-5-yl, benzimidazol-2-yl, pyrid-2-yl, pyrid-3-yl, thien-2-yl, 2,3-dihydro-1,4-benzodioxin-5-yl, 2,3-dihydro-1-benzofuran-7-yl, pyrimidin-5-yl, 1-methylimidazol-2-yl, indol-4-yl, indol-7-yl, 1-methylpyrrol-2-yl or pyrazin-2-yl.

R¹ is not N,N—(C₁₋₆alkyl)₂amino.

R¹ is a substituent on carbon and is selected from halo, nitro, hydroxy, amino, sulphamoyl, C₁₋₆alkyl, C₂₋₆alkynyl, C₁₋₆alkoxy, C₁₋₆alkanoyl, N,N—(C₁₋₆alkyl)₂amino, C₁₋₆alkanoylamino, C₁₋₆alkylS(O)ₐ wherein a is 0 to 2, C₁₋₆alkoxycarbonylamino, N,N—(C₁₋₆alkyl)₂sulphamoyl, C₁₋₆alkylsulphonylamino, carbocyclyl-R¹⁰— or heterocyclyl-R¹¹—; wherein R¹ may be optionally substituted on carbon by one or more R¹²;

R¹² is selected from halo, cyano, hydroxy, C₁₋₆alkyl, C₁₋₆alkoxy, carbocyclyl-R²⁶— or heterocyclyl-R²⁷—; wherein R¹² may be optionally substituted on carbon by one or more R²⁸; and wherein if said heterocyclyl contains an —NH— moiety that nitrogen may be optionally substituted by a group selected from R²⁹;

R¹⁰, R¹¹, R²⁶ and R²⁷ are a direct bond;

R²⁹ is C₁₋₆alkyl;

R²⁸ is selected from hydroxy and methyl.

R¹ is a substituent on carbon and is selected from halo, nitro, hydroxy, amino, sulphamoyl, C₁₋₆alkyl, C₁₋₆alkoxy, C₁₋₆alkanoyl, N,N—(C₁₋₆alkyl)₂amino, C₁₋₆alkanoylamino, C₁₋₆alkylS(O)ₐ wherein a is 0, C₁₋₆alkoxycarbonylamino, C₁₋₆alkylsulphonylamino, carbocyclyl-R¹⁰— or heterocyclyl-R¹¹—; wherein R¹ may be optionally substituted on carbon by one or more R¹²; wherein

R¹² is selected from halo, cyano, C₁₋₆alkyl or carbocyclyl-R²⁶—;

R¹⁰, R¹¹ and R²⁶ are a direct bond.

R¹ is a substituent on carbon and is selected from fluoro, chloro, iodo, nitro, hydroxy, amino, sulphamoyl, methyl, ethyl, propyl, isopropyl, t-butyl, ethynyl, propynyl, 3,3-dimethylprop-1-yn-1-yl, methoxy, ethoxy, propoxy, isopropoxy, butoxy, isobutoxy, acetyl, 2,2-dimethylpropionylamino, dimethylamino, N-methyl-N-ethylamino, acetylamino, methylthio, mesyl, N,N-dimethylsulphamoyl, mesylamino, t-butoxycarbonylamino, cyclopropyl-R¹⁰—, cyclobutyl-R¹⁰—, thienyl-R¹¹—, pyrrolyl-R¹¹—, pyridyl-R¹¹—, piperidinyl-R¹¹—, morpholino-R¹¹—, thiazolyl-R¹¹— or tetrahydro-2H-pyranyl-R¹¹—; wherein R¹ may be optionally substituted on carbon by one or more R¹²;

R¹² is selected from fluoro, cyano, hydroxy, methyl, methoxy, cyclopropyl-R²⁶—, cyclopentyl-R²⁶—, phenyl-R²⁶—, pyrrolidinyl-R²⁷—, piperazinyl-R²⁷— or pyrazolyl-R²⁷—; wherein R¹² may be optionally substituted on carbon by one or more R²⁸; and wherein if said heterocyclyl contains an —NH— moiety that nitrogen may be optionally substituted by a group selected from R²⁹;

R¹⁰, R¹¹, R²⁶ and R²⁷ are a direct bond;

R²⁹ is methyl;

R²⁸ is selected from hydroxy and methyl.

R¹ is a substituent on carbon and is selected from fluoro, chloro, nitro, hydroxy, amino, sulphamoyl, methyl, ethyl, isopropyl, t-butyl, methoxy, propoxy, isopropoxy, isobutoxy, acetyl, dimethylamino, acetylamino, methylthio, t-butoxycarbonylamino, mesylamino, cyclopropyl, cyclobutyl, thienyl, pyrrolyl, pyridinyl, thiazolyl or tetrahydropyranyl; wherein R¹ may be optionally substituted on carbon by one or more R¹²; wherein

R¹² is selected from fluoro, cyano, methyl or phenyl.

R¹ is a substituent on carbon and is selected from fluoro, chloro, iodo, nitro, hydroxy, amino, sulphamoyl, methyl, trifluoromethyl, cyanomethyl, 3,5-dimethylpyrazol-1-ylmethyl, ethyl, 1-methyl-1-cyanoethyl, propyl, isopropyl, t-butyl, (1-hydroxycyclopentyl)ethynyl, cyclopropylethynyl, 3-hydroxyprop-1-yn-1-yl, 3-(1-methylpiperazin-4-yl)prop-1-yn-1-yl, 3-(cyclopentyl)prop-1-yn-1-yl, 3,3-dimethylprop-1-yn-1-yl, benzyloxy, 2-pyrrolidin-1-ylethoxy, propoxy, isopropoxy, butoxy, isobutoxy, methylthio, difluoromethylthio, mesyl, dimethylamino, N-methyl-N-(2-methoxyethyl)amino, acetyl, N,N-dimethylsulphamoyl, acetylamino, t-butoxycarbonylamino, 2,2-dimethylpropionylamino, mesylamino, cyclopropyl, 1-cyanocyclopropyl, 1-cyanocyclobutyl, 1-cyano-tetrahydro-2H-pyran-4-yl, thien-2-yl, pyrrol-1-yl, 2,5-dimethylpyrrol-1-yl, pyrid-3-yl, 2-methylthiazol-4-yl, morpholino andpiperidin-1-yl.

R¹ is a substituent on carbon and is selected from fluoro, chloro, nitro, hydroxy, amino, sulphamoyl, methyl, ethyl, isopropyl, t-butyl, trifluoromethyl, cyanomethyl, 1-methyl-cyanoethyl, propoxy, isopropoxy, isobutoxy, methylthio, acetyl, 1-cyanocyclobutyl, 1-cyanotetrahydropyranyl, 1-cyanocyclopropyl, thien-2-yl, pyrrol-1-yl, pyrid-3-yl, 2-methyl-1,3-thiazol-4-yl, benzyloxy or acetylamino, mesylamino, dimethylamino, t-butoxycarbonylamino.

n is selected from 0-2; wherein the values of R¹ may be the same or different.

n is 0.

n is 1.

n is selected from 2; wherein the values of R¹ may be the same or different.

Y is —C(O)—.

Y is —CH₂—

Z is —C(O)NH—.

Z is —NHC(O)—.

Z is —CH₂NH—.

Z is —C(O)NH— or —CH₂NH—.

R² is selected from hydrogen or halo.

R² is selected from hydrogen or bromo.

R² is selected from hydrogen.

R³ is selected from halo, methyl or methoxy.

R³ is selected from fluoro, chloro, bromo, methyl or methoxy.

R³ is selected from methyl.

X is —NR¹⁸C(O)—.

X is —NHC(O)—.

X is —NR¹⁹—.

X is —NH—.

X is —NR²⁰CH₂—;

X is —NHCH₂—;

X is —NHC(O)—, —NH— or —NHCH₂—.

R⁴, R⁵, R⁶, R⁷ and R⁸ are independently selected from hydrogen, halo, C₁₋₆alkyl, N—(C₁₋₆alkyl)amino, N,N—(C₁₋₆alkyl)₂amino or heterocyclyl-R²²—; wherein R⁴, R⁵, R⁶, R⁷ and R⁸ independently of each other may be optionally substituted on carbon by one or more R²³; wherein

R²³ is selected from hydroxy, amino, N—(C₁₋₆alkyl)amino, N,N—(C₁₋₆alkyl)₂amino or heterocyclyl-R²⁷—; and

R²² and R²⁷ are selected from a direct bond.

R⁴, R⁵, R⁶, R⁷ and R⁸ are independently selected from hydrogen or C₁₋₆alkyl.

R⁴, R⁵, R⁶, R⁷ and R⁸ are independently selected from hydrogen, chloro, methyl, methylamino, ethylamino, propylamino, N-methyl-N-ethylamino, N-methyl-N-propylamino or morpholino-R²²—; wherein R⁴, R⁵, R⁶, R⁷ and R⁸ independently of each other may be optionally substituted on carbon by one or more R²³; wherein

R²³ is selected from hydroxy, amino, methylamino, dimethylamino, morpholino-R²⁷— or piperidin-1-yl-R²⁷—;

R²² and R²⁷ are selected from a direct bond.

R⁴, R⁵, R⁶, R⁷ and R⁸ are independently selected from hydrogen or methyl.

R⁴, R⁵, R⁶, R⁷ and R⁸ are independently selected from hydrogen, chloro, methyl, 3-(piperidin-1-yl)propylamino, 2-hydroxyethylamino, 2-(dimethylamino)ethylamino, 2-(morpholino)ethylamino, methylamino, N-methyl-N-ethylamino, N-methyl-N-(2-methylaminoethyl)amino, morpholino, 3-aminopropylamino or N-methyl-N-(3-dimethylaminopropyl)amino.

R⁴, R⁵ and R⁶ are hydrogen.

R⁷ and R⁸ are independently selected from hydrogen or methyl.

R⁴, R⁵ and R⁶ are hydrogen and R⁷ and R⁸ are independently selected from hydrogen, chloro, methyl, 3-(piperidin-1-yl)propylamino, 2-hydroxyethylamino, 2-(dimethylamino)ethylamino, 2-(morpholino)ethylamino, methylamino, N-methyl-N-ethylamino, N-methyl-N-(2-methylaminoethyl)amino, morpholino, 3-aminopropylamino or N-methyl-N-(3-dimethylaminopropyl)amino.

R⁴, R⁵ and R⁶ are hydrogen and R⁷ and R⁸ are independently selected from hydrogen or methyl.

Therefore in a further aspect of the invention there is provided a compound of formula (Ia) wherein:

Ring A is carbocyclyl or heterocyclyl; wherein if said heterocyclyl contains an —NH— moiety that nitrogen may be optionally substituted by a group selected from R⁹;

R¹ is a substituent on carbon and is selected from halo, nitro, hydroxy, amino, sulphamoyl, C₁₋₆alkyl, C₁₋₆alkoxy, C₁₋₆alkanoyl, N,N—(C₁₋₆alkyl)₂amino, C₁₋₆alkanoylamino, C₁₋₆alkylS(O)ₐ wherein a is 0, C₁₋₆alkoxycarbonylamino, C₁₋₆alkylsulphonylamino, carbocyclyl-R¹⁰— or heterocyclyl-R¹¹—; wherein R¹ may be optionally substituted on carbon by one or more R¹²;

R¹² is selected from halo, cyano, C₁₋₆alkyl or carbocyclyl-R²⁶—;

R¹⁰, R¹¹ and R²⁶ are a direct bond;

n is selected from 0-2; wherein the values of R¹ may be the same or different;

Y is —C(O)— or —CH₂—.

R² is selected from hydrogen or halo;

R² is selected from hydrogen or bromo;

R³ is selected from halo, methyl or methoxy;

X is —NHC(O)—, —NH— or —NHCH₂—;

R⁴, R⁵, R⁶, R⁷ and R⁸ are independently selected from hydrogen or C₁₋₆alkyl.

or a pharmaceutically acceptable salt thereof; with the proviso that said compound is not N-(5-{[3-(dimethylamino)benzoyl]amino}-2-methylphenyl)quinoxaline-6-carboxamide.

Therefore in a further aspect of the invention there is provided a compound of formula (I) wherein:

Ring A is carbocyclyl or heterocyclyl; wherein if said heterocyclyl contains an —NH— moiety that nitrogen may be optionally substituted by a group selected from R⁹;

R¹ is a substituent on carbon and is selected from halo, nitro, hydroxy, amino, sulphamoyl, C₁₋₆alkyl, C₂₋₆alkynyl, C₁₋₆alkoxy, C₁₋₆alkanoyl, N,N—(C₁₋₆alkyl)₂amino, C₁₋₆alkanoylamino, C₁₋₆alkylS(O)ₐ wherein a is 0 to 2, C₁₋₆alkoxycarbonylamino, N,N—(C₁₋₆alkyl)₂sulphamoyl, C₁₋₆alkylsulphonylamino, carbocyclyl-R¹⁰— or heterocyclyl-R¹¹—; wherein R¹ may be optionally substituted on carbon by one or more R¹²;

n is selected from 0-2; wherein the values of R¹ may be the same or different;

Z is —C(O)NH—, —NHC(O)— or —CH₂NH—;

R² is selected from hydrogen or halo;

R³ is selected from halo, methyl or methoxy;

X is —NHC(O)—, —NH— or —NHCH₂—;

R⁴, R⁵, R⁶, R⁷ and R⁸ are independently selected from hydrogen, halo, C₁₋₆alkyl, N—(C₁₋₆alkyl)amino, N,N—(C₁₋₆alkyl)₂amino or heterocyclyl-R²²—; wherein R⁴, R⁵, R⁶, R⁷ and R⁸ independently of each other may be optionally substituted on carbon by one or more R²³;

R⁹ is selected from C₁₋₆alkyl;

R¹² is selected from halo, cyano, hydroxy, C₁₋₆alkyl, C₁₋₆alkoxy, carbocyclyl-R²⁶— or heterocyclyl-R²⁷—; wherein R¹² may be optionally substituted on carbon by one or more R²⁸; and wherein if said heterocyclyl contains an —NH— moiety that nitrogen may be optionally substituted by a group selected from R²⁹;

R²³ is selected from hydroxy, amino, N—(C₁₋₆alkyl)amino, N,N—(C₁₋₆alkyl)₂amino or heterocyclyl-R²⁷—;

R¹⁰, R¹¹, R²², R²⁶ and R²⁷ are a direct bond;

R²⁸ is selected from hydroxy and methyl;

R²⁹ is C₁₋₆alkyl;

or a pharmaceutically acceptable salt thereof; with the proviso that said compound is not N-(5-{[3-(dimethylamino)benzoyl]amino}-2-methylphenyl)quinoxaline-6-carboxamide.

Therefore in a further aspect of the invention there is provided a compound of formula (Ia) wherein:

Ring A is phenyl, 1-t-butylpyrazol-5-yl, benzimidazol-2-yl, pyrid-2-yl, pyrid-3-yl, thien-2-yl, 2,3-dihydro-1,4-benzodioxin-5-yl, 2,3-dihydro-1-benzofuran-7-yl, pyrimidin-5-yl, 1-methylimidazol-2-yl, indol-4-yl, indol-7-yl, 1-methylpyrrol-2-yl or pyrazin-2-yl;

R¹ is a substituent on carbon and is selected from fluoro, chloro, nitro, hydroxy, amino, sulphamoyl, methyl, ethyl, isopropyl, t-butyl, trifluoromethyl, cyanomethyl, 1-methyl-cyanoethyl, propoxy, isopropoxy, isobutoxy, methylthio, acetyl, 1-cyanocyclobutyl, 1-cyanotetrahydropyranyl, 1-cyanocyclopropyl, thien-2-yl, pyrrol-1-yl, pyrid-3-yl, 2-methyl-1,3-thiazol-4-yl, benzyloxy or acetylamino, mesylamino, dimethylamino, t-butoxycarbonylamino;

n is selected from 0-2; wherein the values of R¹ may be the same or different;

Y is —C(O)— or —CH₂—;

R² is selected from hydrogen or bromo;

R³ is selected from fluoro, chloro, bromo, methyl or methoxy;

X is —NHC(O)—, —NH— or —NHCH₂—;

R⁴, R⁵ and R⁶ are hydrogen and R⁷ and R⁸ are independently selected from hydrogen or methyl;

or a pharmaceutically acceptable salt thereof; with the proviso that said compound is not N-(5-{[3-(dimethylamino)benzoyl]amino}-2-methylphenyl)quinoxaline-6-carboxamide.

Therefore in a further aspect of the invention there is provided a compound of formula (I) wherein:

Ring A is phenyl, 1-methylpyrazol-3-yl, 1-methylpyrazol-5-yl, 1-t-butylpyrazol-5-yl, benzimidazol-2-yl, pyrid-2-yl, pyrid-3-yl, pyrid-4-yl, thien-2-yl, thien-3-yl, fur-2-yl, 2,3-dihydro-1,4-benzodioxin-5-yl, 2,3-dihydro-1-benzofuran-7-yl, pyrimidin-4-yl, pyrimidin-5-yl, 1-methylimidazol-2-yl, indol-4-yl, indol-7-yl, 1-methylpyrrol-2-yl or pyrazin-2-yl;

R¹ is a substituent on carbon and is selected from fluoro, chloro, iodo, nitro, hydroxy, amino, sulphamoyl, methyl, trifluoromethyl, cyanomethyl, 3,5-dimethylpyrazol-1-ylmethyl, ethyl, 1-methyl-1-cyanoethyl, propyl, isopropyl, t-butyl, (1-hydroxycyclopentyl)ethynyl, cyclopropylethynyl, 3-hydroxyprop-1-yn-1-yl, 3-(1-methylpiperazin-4-yl)prop-1-yn-1-yl, 3-(cyclopentyl)prop-1-yn-1-yl, 3,3-dimethylprop-1-yn-1-yl, benzyloxy, 2-pyrrolidin-1-ylethoxy, propoxy, isopropoxy, butoxy, isobutoxy, methylthio, difluoromethylthio, mesyl, dimethylamino, N-methyl-N-(2-methoxyethyl)amino, acetyl, N,N-dimethylsulphamoyl, acetylamino, t-butoxycarbonylamino, 2,2-dimethylpropionylamino, mesylamino, cyclopropyl, 1-cyanocyclopropyl, 1-cyanocyclobutyl, 1-cyano-tetrahydro-2H-pyran-4-yl, thien-2-yl, pyrrol-1-yl, 2,5-dimethylpyrrol-1-yl, pyrid-3-yl, 2-methylthiazol-4-yl, morpholino and piperidin-1-yl;

n is selected from 0-2; wherein the values of R¹ may be the same or different;

Z is —C(O)NH—, —NHC(O)— or —CH₂NH—;

R² is selected from hydrogen or bromo;

R³ is selected from fluoro, chloro, bromo, methyl or methoxy;

X is —NHC(O)—, —NH— or —NHCH₂—;

R⁴, R⁵, R⁶, R⁷ and R⁸ are independently selected from hydrogen, chloro, methyl, 3-(piperidin-1-yl)propylamino, 2-hydroxyethylamino, 2-(dimethylamino)ethylamino, 2-(morpholino)ethylamino, methylamino, N-methyl-N-ethylamino, N-methyl-N-(2-methylaminoethyl)amino, morpholino, 3-aminopropylamino or N-methyl-N-(3-dimethylaminopropyl)amino; or a pharmaceutically acceptable salt thereof; with the proviso that said compound is not N-(5-{[3-(dimethylamino)benzoyl]amino}-2-methylphenyl)quinoxaline-6-carboxamide.

In another aspect of the invention, preferred compounds of the invention are any one of the Examples or a pharmaceutically acceptable salt thereof.

In another aspect of the invention, preferred compounds of the invention are any one of Examples 18, 27, 31, 36, 38, 51, 70, 71, 72, 75 or a pharmaceutically acceptable salt thereof.

Another aspect of the present invention provides a process for preparing a compound of formula (I) or a pharmaceutically acceptable salt thereof which process (wherein variable are, unless otherwise specified, as defined in formula (I)) comprises of:

Process a) for compounds of formula (Ia) wherein Y is —C(O)— or compounds of formula (I) wherein Z is —C(O)NH—; reacting an amine of the formula (II)

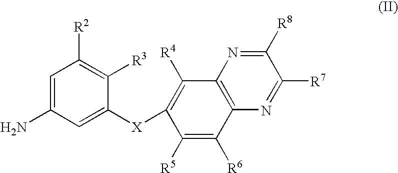

with an acid of formula (III):

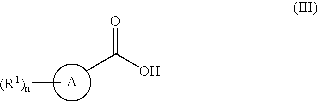

or an activated acid derivative thereof;
Process b) for compounds of formula (I) wherein X is —NR¹⁸C(O)— and R¹⁸ is hydrogen or C₁₋₆alkyl; reacting an amine of formula (IVa) (for compounds of formula (Ia)) or (IV) (for compounds of formula (I)):

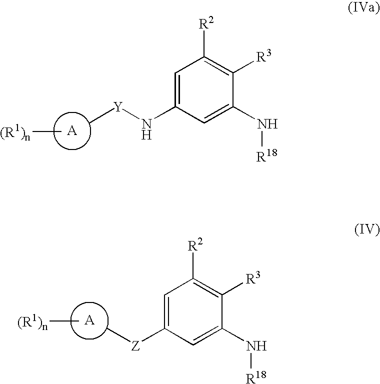

with an acid of formula (V):

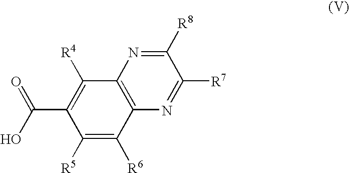

or an activated acid derivative thereof;
Process c) for compounds of formula (Ia) wherein Y is —CH₂— or (I) wherein Z is —CH₂NH—; reacting an amine of the formula (II) with a compound of formula (VI):

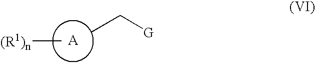

wherein G is a displaceable group;
Process d) for compounds of formula (Ia) wherein Y is —CH₂— or (I) wherein Z is —CH₂NH—; reacting an amine of the formula (VII):

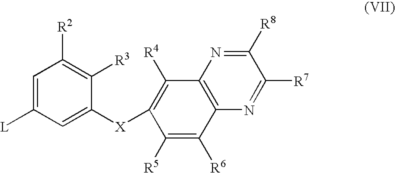

wherein L is a displaceable group; with a compound of formula (VIII):

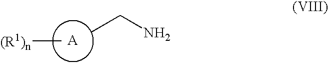

Process e) for compounds of formula (I) wherein X is —NR¹⁹— and R¹⁹ is hydrogen or C₁₋₆alkyl; reacting an amine of formula (IXa) (for compounds of formula (Ia)) or (IX) (for compounds of formula (I)):

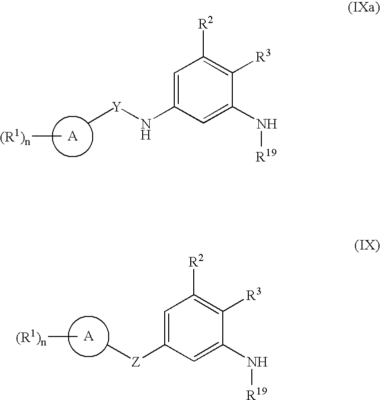

with a compound of formula (X):

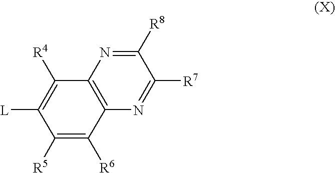

wherein L is a displaceable group
Process f) for compounds of formula (I) wherein X is —NR¹⁹— and R¹⁹ is hydrogen or C₁₋₆alkyl; reacting an amine of formula (XIa) (for compounds of formula (Ia)) or (XI) (for compounds of formula (I)):

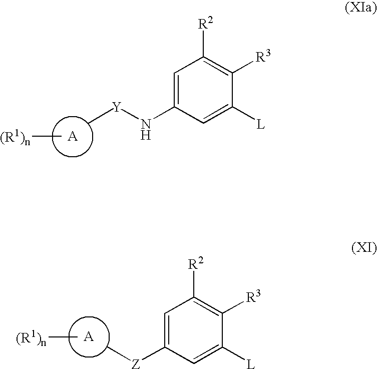

wherein L is a displaceable group; with a compound of formula (XII):

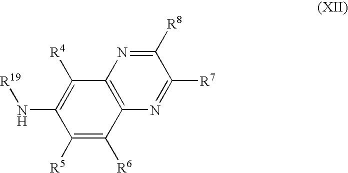

Process g) for compounds of formula (I) wherein X is —NR²⁰CH₂—R²⁰ is hydrogen or C₁₋₆alkyl; reacting an amine of formula (XIIIa) (for compounds of formula (Ia)) or (XIII) (for compounds of formula (I)):

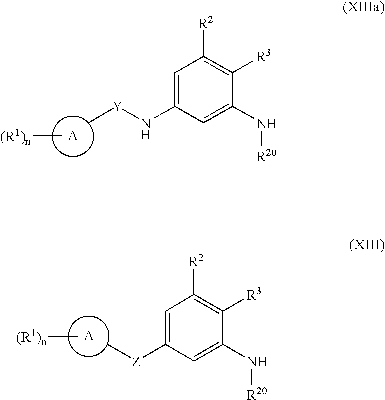

with a compound of formula (XIV):

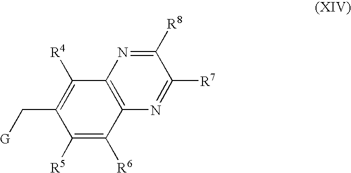

wherein G is a displaceable group;
Process h) for compounds of formula (I) wherein X is —NR²⁰CH₂— wherein R²⁰ is hydrogen or C₁₋₆alkyl; reacting an amine of formula (XIII) (for compounds of formula (I) or (XIIIa) for compounds of formula (Ia) with a compound of formula (XVI):

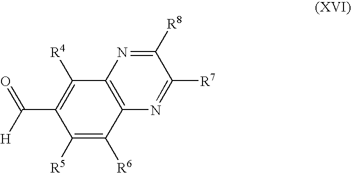

wherein L is a displaceable group
Process i) for compounds of formula (I) wherein Y is —CH₂—; reacting an amine of the formula (II) with a compound of formula (XVII):

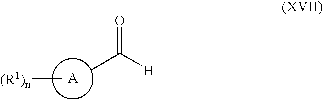

Process j) for compounds of formula (I) (only) where Z is —NHC(O)— reacting a compound of formula (XVIII):

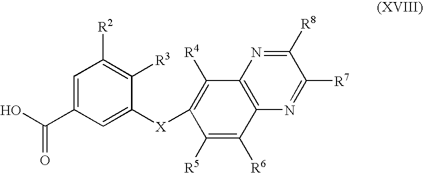

or an activated derivative thereof; with a compound of formula (XIX):

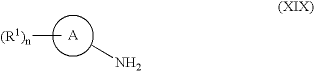

and thereafter if necessary:
i) converting a compound of the formula (I) into another compound of the formula (I);
ii) removing any protecting groups;
iii) forming a pharmaceutically acceptable salt.

L is a displaceable group, suitable values for L are for example, a halo, for example a chloro, bromo or iodo.

G is a displaceable group, suitable values for G are for example, a halo, for example a chloro, bromo or iodo; tosyl or mesyl.

Specific reaction conditions for the above reactions are as follows.

Process a) and Process b) and Process j) Amines of formula (II) and acids of formula (III) and amines of formula (IV) and acids of formula (V) and amines of formula (XIX) and acids of formula (XVIII) may be coupled together in the presence of a suitable coupling reagent. Standard peptide coupling reagents known in the art can be employed as suitable coupling reagents, or for example carbonyldiimidazole and dicyclohexyl-carbodiimide, optionally in the presence of a catalyst such as dimethylaminopyridine or 4-pyrrolidinopyridine, optionally in the presence of a base for example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 50° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 50° C.

Amines of formula (II) may be prepared according to Scheme 1:

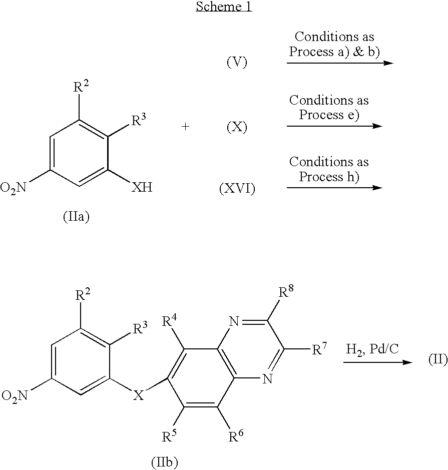

Amines of formula (IV) may be prepared according to Scheme 2:

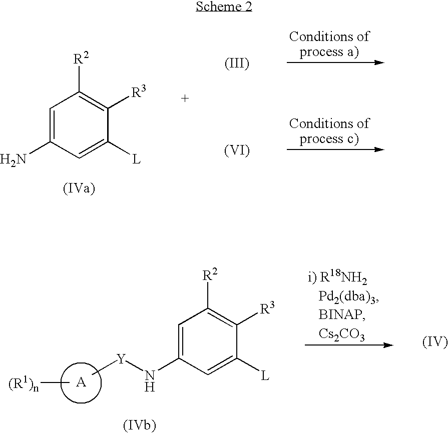

Wherein L is a displaceable group as defined above.

Acids of formula (XVIII) may be prepared according to Scheme 3:

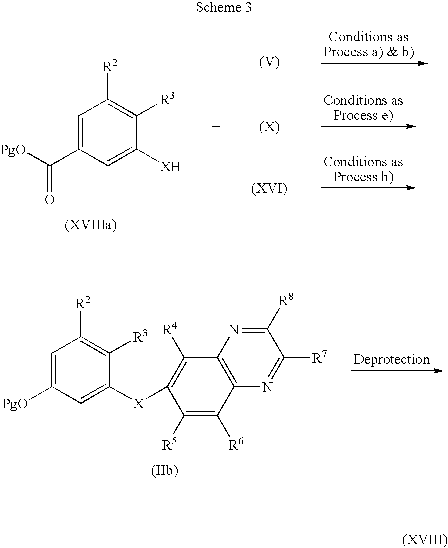

Compounds of formula (IIa), (III), (IVa), (XVIIIa), (XIX) and (V) are commercially available compounds, or they are known in the literature or they may be prepared by standard processes known in the art.

Process c) and Process g) Compounds of formula (II) and (VI) and compounds of formula (XIII) and (XIV) can be reacted together in solvents such as DMF or CH₃CN in the presence of a base such as K₂CO₃ or Cs₂CO₃. The reaction usually requires thermal conditions in the range of 50° C. to 100° C.

Compound (XIII) may be prepared by the process outline for compound (IV) but wherein R¹⁸ is substituted for R²⁰.

Compounds of formula (VI) and (XV) are commercially available compounds, or they are known in the literature or they may be prepared by standard processes known in the art.

Process d), Process e) and Process f) Compounds of formula (VII) and (VIII) and compounds of formula (IX) and (X) and compounds of formula (XI) and (XII) can be reacted together by coupling chemistry utilizing an appropriate catalyst and ligand such as Pd₂(dba)₃ and BINAP respectively and a suitable base such as sodium tert-butoxide. The reaction usually requires thermal conditions often in the range of 80° C. to 100° C.

Compounds of formula (VII) may be prepared according to Scheme 4:

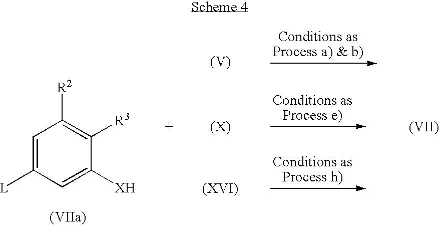

Compound (IX) may be prepared by the process outline for compound (IV) but wherein R¹⁸ is substituted for R¹⁹.

Compounds of formula (XI) may be prepared according to Scheme 5:

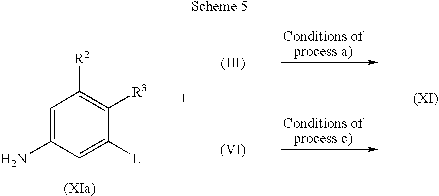

Compounds of formula (VIIa), (VIII), (X), (XIa) and (XII) are commercially available compounds, or they are known in the literature or they may be prepared by standard processes known in the art.

Process h) and Process i) Compounds of the formula (XV) and (XVI) and compounds of the formula (I) and (XVII) in solvents such as THF or 1,2-dichloroethane in the presence of a reducing agent such as sodium triacetoxyborohydride or sodium cyanoborohydride.

Compound (XV) may be prepared by the process outline for compound (IV) but wherein R¹⁸ is substituted for hydrogen.

Compounds of formula (XVI) and (XVII) are commercially available compounds, or they are known in the literature or they may be prepared by standard processes known in the art.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possesses anti-cancer activity which is believed to arise from the B-Raf inhibitory activity of the compound. These properties may be assessed, for example, using the procedure set out below:—

### B-Raf In Vitro ELISA Assay

Activity of human recombinant, purified wild type His-B-Raf protein kinase was determined in vitro using an enzyme-linked immunosorbent assay (ELISA) assay format, which measures phosphorylation of the B-Raf substrate, human recombinant, purified His-derived (detagged) MEK1. The reaction utilized 2.5 nM B-Raf, 0.15 μM MEK1 and 10 μM adenosine triphosphate (ATP) in 40 mM N-(2-Hydroxyethyl)piperazine-N′-(2-ethanesulfonic acid hemisodium salt (HEPES), 5 nM 1,4-Dithio-DL-threitol (DTT), 10 mM MgCl₂, 1 mM ethylenediaminetetraacetic acid (EDTA) and 0.2 M NaCl (1×HEPES buffer), with or without compound at various concentrations, in a total reaction volume of 25 μl in 384 well plates. B-Raf and compound were preincubated in 1×HEPES buffer for 1 hour at 25° C. Reactions were initiated with addition of MEK1 and ATP in 1× HEPES buffer and incubated at 25° C. for 50 minutes and reactions stopped by addition of 10 μl 75 mM EDTA (final concentration 50 mM) in 1×HEPES buffer. 5 μl of the assay mix was then diluted 1:20 into 50 mM EDTA in 1×HEPES buffer, transferred to 384 well black high protein binding plates and incubated overnight at 4° C. Plates were washed in tris buffered saline containing 0.1% Tween20 (TBST), blocked with 50 μl Superblock (Pierce) for 1 hour at 25° C., washed in TBST, incubated with 50 μl rabbit polyclonal anti-phospho-MEK antibody (Cell Signaling) diluted 1:1000 in TBS for 2 hours at 25° C., washed with TBST, incubated with 50 μl goat anti-rabbit horseradish peroxidase-linked antibody (Cell Signaling) diluted 1:2000 in TBS for 1 hour at 25° C. and washed with TBST. 50 μl of fluorogenic peroxidase substrate (Quantablu-Pierce) was added and following incubation for 45-60 minutes, 50 μl QuantabluSTOP (Pierce) was added. Blue fluorescent product was detected at excitation 325 nm and emission 420 nm using a TECAN Ultra plate reader. Data was graphed and IC₅₀s calculated using Excel Fit (Microsoft).

When tested in the above in vitro assay, the compounds of the present invention exhibited activity less than 30 μM. For example the following results were obtained:

| Example No | IC₅₀ (μM) |
|---|---|
| 11 | 742 nM |
| 12 | 20 nM |

According to a further aspect of the invention there is provided a pharmaceutical composition which comprises a compound of the formula (I), or a pharmaceutically acceptable salt thereof, as defined hereinbefore, in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 1-1000 mg/kg, and this normally provides a therapeutically-effective dose. Preferably a daily dose in the range of 10-100 mg/kg is employed. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula (I), or a pharmaceutically acceptable salt thereof, as defined hereinbefore for use in a method of treatment of the human or animal body by therapy.

We have found that the compounds defined in the present invention, or a pharmaceutically acceptable salt thereof, are effective anti-cancer agents which property is believed to arise from their B-Raf inhibitory properties. Accordingly the compounds of the present invention are expected to be useful in the treatment of diseases or medical conditions mediated alone or in part by B-Raf, i.e. the compounds may be used to produce a B-Raf inhibitory effect in a warm-blooded animal in need of such treatment.

Thus the compounds of the present invention provide a method for treating cancer characterised by inhibition of B-Raf, i.e. the compounds may be used to produce an anti-cancer effect mediated alone or in part by the inhibition of B-Raf.

Such a compound of the invention is expected to possess a wide range of anti-cancer properties as activating mutations in B-Raf have been observed in many human cancers, including but not limited to, melanoma, papillary thyroid tumors, cholangiocarcinomas, colon, ovarian and lung cancers. Thus it is expected that a compound of the invention will possess anti-cancer activity against these cancers. It is in addition expected that a compound of the present invention will possess activity against a range of leukaemias, lymphoid malignancies and solid tumours such as carcinomas and sarcomas in tissues such as the liver, kidney, bladder, prostate, breast and pancreas. In particular such compounds of the invention are expected to slow advantageously the growth of primary and recurrent solid tumours of, for example, the skin, colon, thyroid, lungs and ovaries. More particularly such compounds of the invention, or a pharmaceutically acceptable salt thereof, are expected to inhibit the growth of those primary and recurrent solid tumours which are associated with B-Raf, especially those tumours which are significantly dependent on B-Raf for their growth and spread, including for example, certain tumours of the skin, colon, thyroid, lungs and ovaries. Particularly the compounds of the present invention are useful in the treatment of melanomas.

Thus according to this aspect of the invention there is provided a compound of the formula (I), or a pharmaceutically acceptable salt thereof, as defined hereinbefore for use as a medicament.

According to a further aspect of the invention there is provided the use of a compound of the formula (I), or a pharmaceutically acceptable salt thereof, as defined hereinbefore in the manufacture of a medicament for use in the production of a B-Raf inhibitory effect in a warm-blooded animal such as man.

According to this aspect of the invention there is provided the use of a compound of the formula (I), or a pharmaceutically acceptable salt thereof, as defined hereinbefore in the manufacture of a medicament for use in the production of an anti-cancer effect in a warm-blooded animal such as man.

According to a further feature of the invention, there is provided the use of a compound of the formula (I), or a pharmaceutically acceptable salt thereof, as defined herein before in the manufacture of a medicament for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

According to a further feature of this aspect of the invention there is provided a method for producing a B-Raf inhibitory effect in a warm-blooded animal, such as man, in need of such treatment which comprises administering to said animal an effective amount of a compound of formula (I), or a pharmaceutically acceptable salt thereof, as defined above.

According to a further feature of this aspect of the invention there is provided a method for producing an anti-cancer effect in a warm-blooded animal, such as man, in need of such treatment which comprises administering to said animal an effective amount of a compound of formula (I), or a pharmaceutically acceptable salt thereof, as defined above.

According to an additional feature of this aspect of the invention there is provided a method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof as defined herein before.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of a B-Raf inhibitory effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries in a warm-blooded animal such as man.

According to a further aspect of the invention there is provided the use of N-(5-{[3-(dimethylamino)benzoyl]amino}-2-methylphenyl)quinoxaline-6-carboxamide, or a pharmaceutically acceptable salt thereof, in the manufacture of a medicament for use in the production of a B-Raf inhibitory effect in a warm-blooded animal such as man.

According to this aspect of the invention there is provided the use of N-(5-{[3-(dimethylamino)benzoyl]amino}-2-methylphenyl)quinoxaline-6-carboxamide, or a pharmaceutically acceptable salt thereof, in the manufacture of a medicament for use in the production of an anti-cancer effect in a warm-blooded animal such as man.

According to a further feature of the invention, there is provided the use of N-(5-{[3-(dimethylamino)benzoyl]amino}-2-methylphenyl)quinoxaline-6-carboxamide, or a pharmaceutically acceptable salt thereof, in the manufacture of a medicament for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

According to a further feature of this aspect of the invention there is provided a method for producing a B-Raf inhibitory effect in a warm-blooded animal, such as man, in need of such treatment which comprises administering to said animal an effective amount of N-(5-{[3-(dimethylamino)benzoyl]amino}-2-methylphenyl)quinoxaline-6-carboxamide, or a pharmaceutically acceptable salt thereof.

According to a further feature of this aspect of the invention there is provided a method for producing an anti-cancer effect in a warm-blooded animal, such as man, in need of such treatment which comprises administering to said animal an effective amount of N-(5-{[3-(dimethylamino)benzoyl]amino}-2-methylphenyl)quinoxaline-6-carboxamide, or a pharmaceutically acceptable salt thereof.

According to an additional feature of this aspect of the invention there is provided a method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of N-(5-{[3-(dimethylamino)benzoyl]amino}-2-methylphenyl)quinoxaline-6-carboxamide or a pharmaceutically acceptable salt thereof.

In a further aspect of the invention there is provided a pharmaceutical composition which comprises N-(5-{[3-(dimethylamino)benzoyl]amino}-2-methylphenyl)quinoxaline-6-carboxamide, or a pharmaceutically acceptable salt thereof, as defined herein before in association with a pharmaceutically-acceptable diluent or carrier for use in the production of a B-Raf inhibitory effect in a warm-blooded animal such as man.

In a further aspect of the invention there is provided a pharmaceutical composition which comprises N-(5-{[3-(dimethylamino)benzoyl]amino}-2-methylphenyl)quinoxaline-6-carboxamide, or a pharmaceutically acceptable salt thereof, as defined herein before in association with a pharmaceutically-acceptable diluent or carrier for use in the production of an anti-cancer effect in a warm-blooded animal such as man.

In a further aspect of the invention there is provided a pharmaceutical composition which comprises N-(5-{[3-(dimethylamino)benzoyl]amino}-2-methylphenyl)quinoxaline-6-carboxamide, or a pharmaceutically acceptable salt thereof, as defined herein before in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries in a warm-blooded animal such as man.

The B-Raf inhibitory treatment defined hereinbefore may be applied as a sole therapy or may involve, in addition to the compound of the invention, conventional surgery or radiotherapy or chemotherapy. Such chemotherapy may include one or more of the following categories of anti-tumour agents

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutaride, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies;
(x) cell cycle inhibitors including for example CDK inhibitors (eg flavopiridol) and other inhibitors of cell cycle checkpoints (eg checkpoint kinase); inhibitors of aurora kinase and other kinases involved in mitosis and cytokinesis regulation (eg mitotic kinesins); and histone deacetylase inhibitors; and
(xi) endothelin antagonists, including endothelin A antagonists, endothelin B antagonists and endothelin A and B antagonists; for example ZD4054 and ZD1611 (WO 96 40681), atrasentan and YM598.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

In addition to their use in therapeutic medicine, the compounds of formula (I) and their pharmaceutically acceptable salts are also useful as pharmacological tools in the development and standardisation of in vitro and in vivo test systems for the evaluation of the effects of inhibitors of B-Raf in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non limiting examples in which, unless stated otherwise:

(i) temperatures are given in degrees Celsius (° C.); operations were carried out at room or ambient temperature, that is, at a temperature in the range of 18-25° C.;
(ii) organic solutions were dried over anhydrous sodium sulphate; evaporation of solvent was carried out using a rotary evaporator under reduced pressure (600-4000 Pascals; 4.5-30 mmHg) with a bath temperature of up to 60° C.;
(iii) in general, the course of reactions was followed by TLC and reaction times are given for illustration only;
(iv) final products had satisfactory proton nuclear magnetic resonance (NMR) spectra and/or mass spectral data;
(v) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 400 MHz using perdeuterio dimethyl sulphoxide (DMSO-d₆) as solvent unless otherwise indicated;
(vii) chemical symbols have their usual meanings; SI units and symbols are used;
(viii) solvent ratios are given in volume:volume (v/v) terms; and
(ix) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺;
(x) where a synthesis is described as being analogous to that described in a previous example the amounts used are the millimolar ratio equivalents to those used in the previous example;
(xi) the following abbreviations have been used:
HATU O-(7-Azabenzotriazol-1-yl)-N,N,N′,N′-tetramethyluronium hexafluorophosphate;
THF tetrahydrofuran;
DMF N,N-dimethylformamide;
EtOAc ethyl acetate;
Pd₂(dba)₃ Tris(dibenzylideneacetone)dipalladium (0)
BINAP (+/−)-2,2′-Bis(diphenylphosphino)-1,1′-binaphthyl
DIEA N,N-diisopropylethylamine;
DCM dichloromethane; and
DMSO dimethylsulphoxide;
   (xii) “ISCO” refers to normal phase flash column chromatography using 12 g and 40 g pre-packed silica gel cartridges used according to the manufacturers instruction obtained from ISCO, Inc, 4700 superior street Lincoln, Nebr., USA.; and

### Example 1

### N-(2-Methyl-5-{[3-(methylthio)benzoyl]amino}phenyl)quinoxaline-6-carboxamide

N-(5-Amino-2-methylphenyl)quinoxaline-6-carboxamide hydrochloride (Method 4; 100 mg, 0.317 mmol), 3-(methylthio)benzoic acid (59 mg, 0.348 mmol), HATU (145 mg, 0.380 mmol); anhydrous DMF (2 ml) and DIEA (275 μL, 1.585 mmol) were added to a 20 ml scintillation vial. The reaction mixture was shaken overnight at 25° C. Water (10 ml) was added slowly to precipitate the product. The resulting precipitate was washed with water (10 ml), isolated and dried overnight in a vacuum oven at 70° C. to give the title compound 98.4 mg, (73%) as a solid. NMR: 2.25 (s, 3H), 2.54 (s, 3H), 7.27 (d, 1H), 7.46 (d, 2H), 7.62 (d, 1H), 7.71 (t, 1H), 7.79 (s, 1H), 7.89 (s, 1H), 8.25 (d, 1H), 8.37 (d, 1H), 8.77 (s, 1H), 9.17 (d, 2H), 10.32 (d, 2H); m/z: 429.

### Examples 2-75

The following compounds were prepared by the procedure Example 1 using N-(5-amino-2-methylphenyl)quinoxaline-6-carboxamide hydrochloride (Method 4) and the appropriate SM. In some cases, further purification was required (supercritical fluid and/or reverse phase preparatory HPLC).

| Ex. | Compound | NMR | m/z | SM |
|---|---|---|---|---|
| 2 | N-{5-[(3- | 10.34 (s, 1H), 10.21 (s, 1H), | 441 | 3-isopropoxy |
| | Isopropoxy | 9.13-8.98 (m, 2H), 8.77 (s, 1H), 8.37 (d, | | benzoic acid |
| | benzoyl)amino]-2- | 1H), 8.24 (d, 1H), 7.90 (s, 1H), | | |
| | methylphenyl} | 7.61 (d, 1H), 7.55-7.35 (m, 3H), 7.26 (d, | | |
| | quinoxaline-6- | 1H), 7.12 (d, 1H), 4.76-4.64 (m, | | |
| | carboxamide | 1H), 2.24 (s, 3H), 1.24 (d, 6H) | | |
| 3 | N-{5-[(1H-Indol-4- | 11.35 (s, 1H), 10.35 (s, 1H), | 422 | 1H-indole-4- |
| | ylcarbonyl)amino]- | 10.19 (s, 1H), 9.14-8.97 (m, 2H), 8.77 (s, | | carboxylic acid |
| | 2-methylphenyl} | 1H), 8.39 (d, 1H), 8.23 (d, 1H), | | |
| | quinoxaline-6- | 7.95 (s, 1H), 7.67-7.51 (m, 3H), 7.44 (t, | | |
| | carboxamide | 1H), 7.31-7.14 (m, 2H), 6.82 (s, | | |
| | | 1H), 2.25 (s, 3H) | | |
| 4 | N-{5-[(1H-Indol-7- | 11.24 (s, 1H), 10.31 (d, 2H), | 422 | 1H-indole-7- |
| | ylcarbonyl)amino]- | 9.19-8.95 (m, 2H), 8.80 (s, 1H), 8.40 (d, | | carboxylic acid |
| | 2-methylphenyl} | 1H), 8.24 (d, 1H), 8.01 (s, 1H), | | |
| | quinoxaline-6- | 7.88 (d, 1H), 7.80 (d, 1H), 7.67 (d, 1H), | | |
| | carboxamide | 7.42-7.23 (m, 2H), 7.13 (t, 1H), | | |
| | | 6.51 (d, 1H), 2.27 (s, 3H) | | |
| 5 | N-(2-Methyl-5- | 10.34 (d, 2H), 9.14-9.00 (m, 2H), | 387 | 1-methyl-1H- |
| | {[(1-methyl-1H- | 8.76 (s, 1H), 8.37 (d, 1H), 8.24 (d, | | imidazole-2- |
| | imidazol-2-yl) | 1H), 7.98 (s, 1H), 7.55 (d, 1H), | | carboxylic acid |
| | carbonyl]amino} | 7.42 (s, 1H), 7.24 (d, 1H), 7.07 (s, 1H), | | |
| | phenyl)quinoxaline- | 3.98 (s, 3H), 2.23 (s, 3H) | | |
| | 6-carboxamide | | | |
| 6 | N-{5-[(3,5- | 10.33 (s, 1H), 10.19 (s, 1H), | 411 | 3,5-dimethyl |
| | Dimethyl | 9.07 (d, 2H), 8.77 (s, 1H), 8.38 (d, 1H), | | benzoic acid |
| | benzoyl)amino]-2- | 8.25 (d, 1H), 7.88 (s, 1H), 7.64 (d, | | |
| | methylphenyl} | 1H), 7.56 (s, 2H), 7.28 (d, 1H), | | |
| | quinoxaline-6- | 7.19 (s, 1H), 2.35 (s, 6H), 2.25 (s, 3H) | | |
| | carboxamide | | | |
| 7 | N-{5-[(2,3- | 10.44 (s, 1H), 9.87 (s, 1H), 9.17 (d, | 425 | 2,3-dihydro-1- |
| | Dihydro-1- | 2H), 8.88 (s, 1H), 8.48 (d, 1H), | | benzofuran-7- |
| | benzofuran-7-yl | 8.34 (d, 1H), 7.91 (s, 1H), 7.74-7.62 (m, | | carboxylic acid |
| | carbonyl)amino]-2- | 2H), 7.54 (d, 1H), 7.36 (d, 1H), | | |
| | methylphenyl} | 7.07 (t, 1H), 4.84 (t, 2H), 3.36 (t, 2H), | | |
| | quinoxaline-6- | 2.35 (s, 3H) | | |
| | carboxamide | | | |
| 8 | N-(5-{[(2,2- | 10.40 (s, 1H), 9.73 (s, 1H), | 453 | 2,2-dimethyl-2,3- |
| | Dimethyl-2,3- | 9.17-8.96 (m, 2H), 8.77 (s, 1H), 8.38 (d, | | dihydro-1- |
| | dihydro-1- | 1H), 8.23 (d, 1H), 7.83 (s, 1H), | | benzofuran-7- |
| | benzofuran-7-yl) | 7.63 (d, 1H), 7.49 (d, 1H), 7.40 (d, 1H), | | carboxylic acid |
| | carbonyl]amino}- | 7.28 (d, 1H), 6.97 (t, 1H), 3.11 (s, | | |
| | 2-methylphenyl) | 2H), 2.24 (s, 3H), 1.53 (s, 6H) | | |
| | quinoxaline-6- | | | |
| | carboxamide | | | |
| 9 | N-{5-[(3-Acetyl | 10.50 (s, 1H), 10.38 (s, 1H), | 425 | 3-acetylbenzoic |
| | benzoyl)amino]-2- | 9.16-8.97 (m, 2H), 8.78 (s, 1H), | | acid |
| | methylphenyl} | 8.53-8.47 (m, 1H), 8.35 (d, 1H), | | |
| | quinoxaline-6- | 8.28-8.13 (m, 3H), 7.91 (d, 1H), | | |
| | carboxamide | 7.73-7.60 (m, 2H), 7.99 (d, 1H), 2.66 (s, | | |
| | | 3H), 2.27 (s, 3H) | | |
| 10 | N-(2-Methyl-5- | 10.44 (s, 1H), 10.36 (s, 1H), | 398 | 5-methylnicotinic |
| | {[(5-methyl | 9.16-9.00 (m, 2H), 8.92 (s, 1H), 8.78 (d, | | acid |
| | pyridin-3-yl) | 1H), 8.59 (s, 1H), 8.43-8.33 (m, | | |
| | carbonyl]amino} | 1H), 8.24 (d, 1H), 8.12 (s, 1H), | | |
| | phenyl)quinoxaline- | 7.89 (d, 1H), 7.68-7.55 (m, 1H), 7.29 (d, | | |
| | 6-carboxamide | 1H), 2.28 (s, 3H), 2.25 (s, 3H) | | |
| 11 | N-{5-[(3-Ethyl | 10.35 (s, 1H), 10.23 (s, 1H), | 411 | 3-ethylbenzoic |
| | benzoyl)amino]-2- | 9.19-8.96 (m, 2H), 8.77 (s, 1H), 8.36 (d, | | acid |
| | methylphenyl} | 1H), 8.24 (d, 1H), 7.96-7.75 (m, | | |
| | quinoxaline-6- | 3H), 7.61 (d, 1H), 7.42 (d, 2H), | | |
| | carboxamide | 7.27 (d, 1H), 2.75-2.63 (m, 2H), 2.25 (s, | | |
| | | 3H), 1.23 (t, 3H) | | |
| 12 | N-{2-Methyl-5- | 10.34 (s, 1H), 10.19 (s, 1H), | 441 | 3-propoxybenzoic |
| | [(3-propoxy | 9.07 (d, 2H), 8.76 (s, 1H), 8.39 (d, 1H), | | acid |
| | benzoyl)amino] | 8.24 (d, 1H), 7.90 (s, 1H), 7.63 (d, | | |
| | phenyl} | 1H), 7.56-7.47 (m, 2H), 7.42 (t, | | |
| | quinoxaline-6- | 1H), 7.27 (d, 1H), 7.13 (d, 1H), | | |
| | carboxamide | 3.99 (t, 2H), 2.24 (s, 3H), 1.82-1.69 (m, | | |
| | | 2H), 0.99 (t, 3H) | | |
| 13 | N-{2-Methyl-5- | 10.66 (s, 1H), 10.35 (s, 1H), 9.40 (s, | 385 | pyrimidine-5- |
| | [(pyrimidin-5- | 1H), 9.26 (s, 2H), 9.14-8.99 (m, | | carboxylic acid |
| | ylcarbonyl)amino] | 2H), 8.77 (s, 1H), 8.38 (d, 1H), | | |
| | phenyl} | 8.20 (d, 1H), 7.89 (s, 1H), 7.60 (d, 1H), | | |
| | quinoxaline-6- | 7.32 (d, 1H), 2.28 (s, 3H) | | |
| | carboxamide | | | |
| 14 | N-(2-Methyl-5- | 10.37 (s, 2H), 9.10 (d, 2H), 8.78 (s, | 448 | 3-(1H-pyrrol-1- |
| | {[3-(1H-pyrrol-1- | 1H), 8.38 (d, 1H), 8.24 (d, 1H), | | yl)benzoic acid |
| | yl)benzoyl] | 8.11 (s, 1H), 7.91 (s, 1H), 7.80 (d, 2H), | | |
| | amino}phenyl) | 7.68-7.55 (m, 2H), 7.48 (s, 2H), | | |
| | quinoxaline-6- | 7.30 (d, 1H), 6.30 (s, 2H), 2.27 (s, | | |
| | carboxamide | 3H) | | |
| 15 | N-{2-Methyl-5- | 10.33 (d, 2H), 9.14-8.84 (m, 3H), | 460 | 3-pyridin-3- |
| | [(3-pyridin-3- | 8.72 (s, 1H), 8.56 (d, 1H), | | ylbenzoic acid |
| | ylbenzoyl)amino] | 8.38-8.10 (m, 4H), 7.99-7.84 (m, 3H), | | |
| | phenyl} | 7.67-7.54 (m, 2H), 7.52-7.43 (m, 1H), | | |
| | quinoxaline-6- | 7.22 (d, 1H), 2.21 (s, 3H) | | |
| | carboxamide | | | |
| 16 | N-(2-Methyl-5- | 10.38 (d, 2H), 9.16-8.96 (m, 2H), | 480 | 3-(2-methyl-1,3- |
| | {[3-(2-methyl-1,3- | 8.80 (s, 1H), 8.49 (s, 1H), 8.38 (d, | | thiazol-4- |
| | thiazol-4- | 1H), 8.24 (d, 1H), 8.14 (d, 1H), | | yl)benzoic acid |
| | yl)benzoyl]amino} | 8.06 (d, 1H), 7.91 (d, 2H), 7.68-7.53 (m, | | |
| | phenyl)quinoxaline- | 2H), 7.28 (d, 1H), 2.73 (s, 3H), | | |
| | 6-carboxamide | 2.26 (s, 3H) | | |
| 17 | N-(5-{[3-(Amino | 10.56 (s, 1H), 10.37 (s, 1H), | 462 | 3-(aminosulfonyl) |
| | sulfonyl)benzoyl] | 9.18-8.99 (m, 2H), 8.78 (s, 1H), | | benzoic acid |
| | amino}-2-methyl | 8.44-8.34 (m, 2H), 8.29-8.15 (m, 2H), | | |
| | phenyl)quinoxaline- | 8.00 (d, 1H), 7.90 (s, 1H), 7.73 (t, | | |
| | 6-carboxamide | 1H), 7.63 (d, 1H), 7.50 (s, 2H), | | |
| | | 7.30 (d, 1H), 2.26 (s, 3H) | | |
| 18 | N-{5-[(3,5-Di-tert- | 10.36 (s, 1H), 10.20 (s, 1H), | 495 | 3,5-di-tert- |
| | butylbenzoyl) | 9.14-8.99 (m, 2H), 8.77 (s, 1H), 8.38 (d, | | butylbenzoic acid |
| | amino]-2-methyl | 1H), 8.24 (d, 1H), 7.85 (s, 1H), | | |
| | phenyl} | 7.75 (d, 2H), 7.68-7.57 (m, 2H), 7.29 (d, | | |
| | quinoxaline-6- | 1H), 2.26 (s, 3H), 1.33 (s, 18H) | | |
| | carboxamide | | | |
| 19 | N-{5-[(3-Isobutoxy | 10.33 (s, 1H), 10.23 (s, 1H), | 455 | 3-isobutoxy |
| | benzoyl)amino]-2- | 9.13-9.01 (m, 2H), 8.78 (s, 1H), 8.38 (d, | | benzoic acid |
| | methylphenyl} | 1H), 8.24 (d, 1H), 7.88 (s, 1H), | | |
| | quinoxaline-6- | 7.66-7.37 (m, 4H), 7.28 (d, 1H), | | |
| | carboxamide | 7.14 (d, 1H), 3.81 (d, 2H), 2.27 (s, | | |
| | | 3H), 2.32-2.22 (m, 1H), 1.00 (d, | | |
| | | 6H) | | |
| 20 | N-{5-[(1H- | 13.26 (s, 1H), 10.77 (s, 1H), | 421 | 1H- |
| | Benzimidazol-2- | 10.29 (s, 1H), 8.93 (d, 2H), 8.66 (s, 1H), | | benzimidazole-2- |
| | ylcarbonyl)amino]- | 8.27 (d, 1H), 8.12 (d, 1H), 7.93 (s, | | carboxylic acid |
| | 2-methylphenyl} | 1H), 7.73-7.54 (m, 2H), 7.45 (d, | | |
| | quinoxaline-6- | 1H), 7.27-7.13 (m, 3H), 2.13 (s, 3H) | | |
| | carboxamide | | | |
| 21 | N-{2-Methyl-5- | 10.48 (s, 1H), 10.36 (s, 1H), 9.16 (s, | 38 | nicotinic acid |
| | [(pyridin-3-yl | 1H), 9.04 (d, 2H), 8.82-8.68 (m, | | |
| | carbonyl)amino] | 2H), 8.44-8.20 (m, 3H), 7.90 (s, | | |
| | phenyl} | 1H), 7.67-7.52 (m, 2H), 7.29 (d, | | |
| | quinoxaline-6- | 1H), 2.26 (s, 3H) | | |
| | carboxamide | | | |
| 22 | N-{5-[(2,2′- | 10.30 (d, 2H), 9.05 (d, 2H), 8.77 (s, | 471 | 2,2′-bithiophene- |
| | Bithien-5- | 1H), 8.38 (d, 1H), 8.24 (d, 1H), | | 5-carboxylic acid |
| | ylcarbonyl)amino]- | 8.00 (d, 1H), 7.83 (s, 1H), 7.63-7.56 (m, | | |
| | 2-methylphenyl} | 2H), 7.46 (d, 1H), 7.40 (d, 1H), | | |
| | quinoxaline-6- | 7.28 (d, 1H), 7.13 (d, 1H), 2.27 (s, 3H) | | |
| | carboxamide | | | |
| 23 | N-{5-[(2,3- | 10.34 (s, 1H), 10.11 (s, 1H), | 441 | 2,3-dihydro-1,4- |
| | Dihydro-1,4- | 9.05 (d, 2H), 8.77 (s, 1H), 8.39 (d, 1H), | | benzodioxine-5- |
| | benzodioxin-5- | 8.22 (d, 1H), 7.84 (s, 1H), 7.54 (d, | | carboxylic acid |
| | ylcarbonyl)amino]- | 1H), 7.25 (d, 1H), 7.13 (d, 1H), | | |
| | 2-methylphenyl} | 7.04-6.87 (m, 2H), 4.36 (t, 2H), | | |
| | quinoxaline-6- | 4.29 (t, 2H), 2.25 (s, 3H) | | |
| | carboxamide | | | |
| 24 | N-(2-Methyl-5- | 10.30 (s, 1H), 9.75 (s, 1H), 9.07 (d, | 386 | 1-methyl-1H- |
| | {[(1-methyl-1H- | 2H), 8.77 (s, 1H), 8.38 (d, 1H), | | pyrrole-2- |
| | pyrrol-2-yl) | 8.23 (d, 1H), 7.84 (s, 1H), 7.53 (d, 1H), | | carboxylic acid |
| | carbonyl]amino} | 7.23 (d, 1H), 7.04 (d, 1H), 6.97 (s, | | |
| | phenyl)quinoxaline- | 1H), 6.08 (t, 1H), 3.87 (s, 3H), | | |
| | 6-carboxamide | 2.25 (s, 3H) | | |
| 25 | N-{2-Methyl-5- | 10.70 (s, 1H), 10.37 (s, 1H), 9.30 (s, | 385 | pyrazine-2- |
| | [(pyrazin-2-yl | 1H), 9.07 (d, 2H), 8.92 (d, 1H), | | carboxylic acid |
| | carbonyl)amino] | 8.78 (d, 2H), 8.38 (d, 1H), 8.22 (d, 1H), | | |
| | phenyl}quinoxaline- | 8.05 (s, 1H), 7.72 (d, 1H), 7.30 (d, | | |
| | 6-carboxamide | 1H), 2.25 (s, 3H) | | |
| 26 | N-(5-{[3-(2,5- | 10.32 (d, 2H), 9.05 (d, 2H), 8.77 (s, | 476 | 3-(2,5-dimethyl- |
| | Dimethyl-1H- | 1H), 8.36 (d, 1H), 8.24 (d, 1H), | | 1H-pyrrol-1- |
| | pyrrol-1-yl) | 8.06 (d, 1H), 7.91 (t, 2H), 7.72-7.60 (m, | | yl)benzoic acid |
| | benzoyl]amino}-2- | 2H), 7.50 (d, 1H), 7.27 (d, 1H), | | |
| | methylphenyl) | 5.83 (s, 2H), 2.25 (s, 3H), 1.98 (s, 6H) | | |
| | quinoxaline-6- | | | |
| | carboxamide | | | |
| 27 | N-(5-{[3-(1-Cyano | 10.36 (s, 1H), 10.34 (s, 1H), 9.07 (s, | 462 | Method 74 |
| | cyclobutyl) | 1H), 9.06 (s, 1H), 8.78 (s, 1H), | | |
| | benzoyl]amino}-2- | 8.38 (d, 1H), 8.24 (d, 1H), 7.99 (s, 1H), | | |
| | methylphenyl) | 7.95 (d, 1H), 7.88 (s, 1H), 7.64 (m, | | |
| | quinoxaline-6- | 3H), 7.29 (d, 1H), 2.74 (m, 4H), | | |
| | carboxamide | 2.32 (m, 1H), 2.27 (s, 3H), 2.03 (m, | | |
| | | 1H) | | |
| 28 | N-(5-{[3-(4- | 10.34 (s, 2H), 9.07 (s, 1H), 9.06 (s, | 492 | Method 75 |
| | Cyanotetrahydro- | 1H), 8.77 (s, 1H), 8.37 (d, 1H), | | |
| | 2H-pyran-4- | 8.24 (d, 1H), 8.08 (s, 1H), 7.98 (d, 1H), | | |
| | yl)benzoyl] | 7.87 (s, 1H), 7.78 (d, 1H), 7.63 (m, | | |
| | amino}-2-methyl | 3H), 7.29 (d, 1H), 4.05 (m, 2H), | | |
| | phenyl)quinoxaline- | 3.68 (m, 2H), 2.27 (s, 3H), 2.15 (m, | | |
| | 6-carboxamide | 4H) | | |
| 29 | N-(5-{[3-(1-Cyano | 10.35 (m, 2H), 9.07 (s, 1H), 9.06 (s, | 448 | Method 76 |
| | cyclopropyl) | 1H), 8.77 (s, 1H), 8.37 (d, 1H), | | |
| | benzoyl]amino}-2- | 8.24 (d, 1H), 7.87 (m, 3H), 7.62 (d, 1H), | | |
| | methylphenyl) | 7.56 (m, 2H), 7.28 (d, 1H), 2.26 (s, | | |
| | quinoxaline-6- | 3H), 1.80 (m, 2H), 1.63 (m, 2H) | | |
| | carboxamide | | | |
| 30 | N-{5-[(3-Isopropyl | 10.34 (s, 1H), 10.23 (s, 1H), 9.07 (s, | 425 | Method 72 |
| | benzoyl)amino]-2- | 1H), 9.06 (s, 1H), 8.77 (s, 1H), | | |
| | methylphenyl} | 8.38 (d, 1H), 8.24 (d, 1H), 7.89 (s, 1H), | | |
| | quinoxaline-6- | 7.82 (s, 1H), 7.77 (d, 1H), 7.63 (d, | | |
| | carboxamide | 1H), 7.45 (m, 2H), 7.27 (d, 1H), | | |
| | | 2.98 (m, 1H), 2.26 (s, 3H), 1.25 (d, | | |
| | | 6H) | | |
| 31 | N-(5-{[3-(1- | 10.30 (s, 2H), 8.96-9.05 (m, 2H), | 450 | Method 73 |
| | Cyano-1-methyl | 8.72 (d, 1H), 8.32 (dd, 1H), 8.19 (d, | | |
| | ethyl)benzoyl] | 1H), 7.95-8.02 (m, 1H), 7.89 (d, | | |
| | amino}-2-methyl | 1H), 7.82 (d, 1H), 7.69 (d, 1H), | | |
| | phenyl)quinoxaline- | 7.48-7.62 (m, 2H), 7.23 (d, 1H), 2.21 (s, | | |
| | 6-carboxamide | 3H), 1.69 (s, 6H) | | |
| 32 | N-[5-({[5-(1- | 10.34 (s, 1H), 10.32 (s, 1H), 9.07 (s, | 456 | Method 48 |
| | Cyano-1-methyl | 2H), 8.77 (d, 1H), 8.30 (dd, 1H), | | |
| | ethyl)-2-thienyl] | 8.26 (dd, 1H), 7.96 (s, 1H), 7.83 (s, | | |
| | carbonyl}amino)- | 1H), 7.57 (d, 1H), 7.29 (m, 2H), | | |
| | 2-methylphenyl] | 2.26 (s, 3H), 1.77 (s, 6H) | | |
| | quinoxaline-6- | | | |
| | carboxamide | | | |
| 33 | N-(5-{[4-Chloro-3- | 10.43 (s, 1H), 10.35 (s, 1H), | 484 | Method 79 |
| | (1-cyano-1-methyl | 9.07 (q, 2H), 8.77 (d, 1H), 8.37 (dd, 1H), | | |
| | ethyl)benzoyl] | 8.25 (d, 1H), 8.02 (s, 1H), 7.98 (d, | | |
| | amino}-2-methyl | 1H), 7.86 (s, 1H), 7.74 (d, 1H), | | |
| | phenyl)quinoxaline- | 7.60 (dd, 1H), 7.30 (d, 1H), 2.27 (s, 3H), | | |
| | 6-carboxamide | 1.87 (s, 6H) | | |
| 34 | N-(5-{[4-Chloro-3- | 10.65 (s, 1H), 10.57 (s, 1H), 9.29 (s, | 456 | Method 78 |
| | (cyanomethyl) | 2H), 8.98 (s, 1H), 8.57 (d, 1H), | | |
| | benzoyl]amino}-2- | 8.47 (d, 1H), 8.33 (s, 1H), 8.21 (d, 1H), | | |
| | methylphenyl) | 8.09 (s, 1H), 7.95 (d, 1H), 7.81 (dd, | | |
| | quinoxaline-6- | 1H), 7.52 (d, 1H), 4.41 (s, 2H), | | |
| | carboxamide | 2.27 (s, 3H) | | |
| 35 | N-[5-({[6-(1- | 10.40 (s, 1H), 10.32 (s, 1H), | 451 | Method 52 |
| | Cyano-1-methyl | 9.07 (d, 2H), 8.37 (s, 1H), 8.26 (d, 1H), | | |
| | ethyl)pyridin-2-yl] | 8.13 (d, 1H), 8.11 (m, 2H), 7.90 (m, | | |
| | carbonyl}amino)- | 2H), 7.70 (d, 1H), 7.35 (d, 1H), | | |
| | 2-methylphenyl] | 2.27 (s, 3H), 1.83 (s, 6H) | | |
| | quinoxaline-6- | | | |
| | carboxamide | | | |
| 36 | N-(5-{[3-(Benzyloxy)- | 8.87 (s, 2H), 8.60 (s, 1H), | 556 | Method 77 |
| | 5-(1-cyano-1- | 8.11-8.24 (m, 3H), 8.02 (s, 1H), 7.62 (d, | | |
| | methylethyl) | 1H), 7.45 (s, 1H), 7.27-7.38 (m, | | |
| | benzoyl]amino}-2- | 7H), 7.19 (s, 1H), 7.13 (d, 1H), | | |
| | methylphenyl) | 5.04 (s, 2H), 2.24 (s, 3H), 1.66 (s, 6H) | | |
| | quinoxaline-6- | | | |
| | carboxamide | | | |
| 37 | N-(5-{[3-(1- | 10.15 (s, 1H), 10.06 (s, 1H), 9.83 (s, | 466 | Method 82 |
| | Cyano-1-methyl | 1H), 8.86-8.89 (m, 2H), 8.57 (s, | | |
| | ethyl)-5-hydroxy | 1H), 8.03-8.20 (m, 2H), | | |
| | benzoyl]amino}-2- | 7.65-7.67 (m, 1H), 7.41 (d, 1H), 7.28 (s, | | |
| | methylphenyl) | 1H), 7.06-7.10 (m, 2H), 6.91 (s, | | |
| | quinoxaline-6- | 1H), 2.06 (s, 3H), 1.51 (s, 6H) | | |
| | carboxamide | | | |
| 38 | N-(5-{[3-(1- | 8.72 (s, 1H), 8.58-8.60 (m, 2H), | 464 | Method 81 |
| | Cyano-1-methyl | 8.26 (d, 2H), 8.04 (s, 1H), 7.97 (d, | | |
| | ethyl)-5-methyl | 1H), 7.70-7.75 (m, 3H), 7.50 (t, | | |
| | benzoyl]amino}-2- | 1H), 7.40-7.44 (m, 1H), | | |
| | methylphenyl) | 7.22-7.26 (m, 1H), 7.08 (d, 1H), 2.72 (s, 3H), | | |
| | quinoxaline-6- | 2.33 (s, 6H), 1.98 (s, 3H) | | |
| | carboxamide | | | |
| 39 | N-(5-{[3-(1- | 8.89 (s, 2H), 8.61 (s, 1H), | 468 | Method 80 |
| | Cyano-1-methyl | 8.17-8.25 (m, 2H), 8.14 (bs, 1H), 8.03 (bs, | | |
| | ethyl)-4-fluoro | 1H), 7.98 (bs, 1H), 7.93-7.96 (m, | | |
| | benzoyl]amino}-2- | 2H), 7.76-7.80 (m, 1H), 7.58 (d, | | |
| | methylphenyl) | 1H), 7.11-7.19 (m, 2H), 2.28 (s, | | |
| | quinoxaline-6- | 3H), 1.77 (s, 6H) | | |
| | carboxamide | | | |
| 40 | N-(5-{[4-Fluoro-3- | 8.94 (s, 2H), 8.68 (s, 1H), | 469 | 4-fluoro-3- |
| | (trifluoromethyl) | 8.17-8.35 (m, 5H), 7.82 (s, 1H), 7.41-7.55 (m, | | (trifluoromethyl)benzoic |
| | benzoyl]amino}-2- | 2H), 7.26 (d, 1H), 1.97 (s, 3H) | | acid |
| | methylphenyl) | | | |
| | quinoxaline-6- | | | |
| | carboxamide | | | |
| 41 | N-(5-{[3-(1- | 10.35 (s, 1H), 10.21 (s, 1H), | 493 | Method 83 |
| | Cyano-1-methyl | 9.12-9.00 (m, 2H), 8.78 (s, 1H), | | |
| | ethyl)-5- | 8.43-8.32 (m, 1H), 8.26 (d, 1H), 7.85 (s, | | |
| | (dimethylamino) | 1H), 7.68-7.59 (m, 1H), | | |
| | benzoyl]amino}-2- | 7.35-7.25 (m, 2H), 7.23-7.18 (m, 1H), 6.96 (d, | | |
| | methylphenyl) | 1H), 3.33 (d, 6H), 2.28 (s, 3H), | | |
| | quinoxaline-6- | 1.74 (s, 6H) | | |
| | carboxamide | | | |
| 42 | N-[5-({3-(1- | 10.31 (s, 1H), 10.29 (s, 1H), | 543 | Method 85 |
| | Cyano-1- | 10.05 (s, 1H), 8.97-9.04 (m, 2H), 8.72 (d, | | |
| | methylethyl)-5- | 1H), 8.32 (dd, 1H), 8.19 (d, 1H), | | |
| | [(methylsulfonyl) | 7.78 (d, 1H), 7.69-7.74 (m, 1H), | | |
| | amino]benzoyl} | 7.63-7.68 (m, 1H), 7.56 (dd, 1H), | | |
| | amino)-2-methyl | 7.46-7.53 (m, 1H), 7.22 (d, 1H), | | |
| | phenyl]quinoxaline- | 3.02 (s, 3H), 2.22 (s, 3H), 1.68 (s, | | |
| | 6-carboxamide | 6H) | | |
| 43 | N-(5-{[3- | 10.36 (s, 1H), 10.34 (s, 1H), | 507 | Method 86 |
| | (Acetylamino)-5- | 10.27 (s, 1H), 9.03-9.10 (m, 1.88, 2H), | | |
| | (1-cyano-1- | 8.77 (d, 1H), 8.33-8.41 (m, 1H), | | |
| | methylethyl) | 8.24 (d, 1H), 8.07-8.13 (m, 1H), | | |
| | benzoyl]amino}-2- | 7.96-8.01 (m, 1H), 7.81-7.87 (m, | | |
| | methylphenyl) | 1H), 7.66-7.71 (m, 1H), 7.61 (dd, | | |
| | quinoxaline-6- | 1H), 7.25 (d, 1H), 2.27 (s, 3H), | | |
| | carboxamide | 2.07 (s, 3H), 1.73 (s, 6H) | | |
| 44 | tert-Butyl {3-(1- | 8.98 (s, 2H), 8.62 (s, 1H), | 565 | Method 84 |
| | cyano-1-methyl | 8.31-8.40 (m, 1H), 8.18-8.30 (m, 3H), | | |
| | ethyl)-5-[({4- | 7.91 (s, 1H), 7.70 (s, 1H), 7.65 (s, | | |
| | methyl-3- | 2H), 7.19-7.32 (m, 2H), 6.87 (s, | | |
| | [(quinoxalin-6- | 1H), 2.84 (s, 3H), 1.78 (s, 6H), | | |
| | ylcarbonyl)amino] | 1.55 (s, 9H) | | |
| | phenyl}amino) | | | |
| | carbonyl]phenyl} | | | |
| | carbamate | | | |
| 45 | N-[5-({[4-(1- | 10.33 (s, 2H), 9.07 (d, 2H), 8.77 (d, | 456 | Method 96 |
| | Cyano-1-methyl | 1H), 8.36 (dd, 1H), 8.26-8.20 (m, | | |
| | ethyl)-2-thienyl] | 2H), 7.84-7.83 (m, 2H), 7.59 (dd, | | |
| | carbonyl}amino)- | 1H), 7.29 (d, 1H), 2.27 (s, 3H), | | |
| | 2-methylphenyl] | 1.71 (s, 6H) | | |
| | quinoxaline-6- | | | |
| | carboxamide | | | |
| 46 | N-[5-({[5-(1- | 10.15 (s, 1H), 9.93 (s, 1H), | 456 | Method 49 |
| | Cyano-1-methyl | 8.89-8.87 (m, 2H), 8.58 (d, 1H), | | |
| | ethyl)-3-thienyl] | 8.20 (dd, 1H), 8.06 (d, 1H), 7.75 (s, 1H), | | |
| | carbonyl}amino)- | 7.64 (d, 1H), 7.52 (d, 1H), 7.42 (dd, | | |
| | 2-methylphenyl] | 1H), 7.08 (d, 1H), 2.07 (s, 3H), | | |
| | quinoxaline-6- | 1.61 (s, 6H) | | |
| | carboxamide | | | |
| 47 | N-(5-{[5-(1- | 10.16 (s, 1H), 9.98 (s, 1H), 8.88 (d, | 441 | Method 95 |
| | Cyano-1-methyl | 2H), 8.58 (s, 1H), 8.20 (d, 1H), | | |
| | ethyl)-2-furoyl] | 8.06 (d, 1H), 7.62 (s, 1H), 7.44 (d, 1H), | | |
| | amino}-2-methyl | 7.15 (d, 1H), 7.10 (d, 1H), 6.50 (d, | | |
| | phenyl)quinoxaline- | 1H), 2.08 (s, 3H), 1.56 (s, 6H) | | |
| | 6-carboxamide | | | |
| 48 | N-[5-({[3-(1- | 10.34 (s, 1H), 10.31 (s, 1H), | 455 | Method 93 |
| | Cyano-1-methyl | 9.07 (q, 2H), 8.77 (d, 1H), 8.37 (dd, 1H), | | |
| | ethyl)-1-methyl- | 8.25 (d, 1H), 7.88 (d, 1H), 7.57 (dd, | | |
| | 1H-pyrazol-5-yl] | 1H), 7.29 (d, 1H), 7.17 (s, 1H), | | |
| | carbonyl}amino)- | 4.07 (s, 3H), 2.26 (s, 3H), 1.68 (s, 6H) | | |
| | 2-methylphenyl] | | | |
| | quinoxaline-6- | | | |
| | carboxamide | | | |
| 49 | N-[5-({[5-(1- | 10.18 (s, 1H), 9.94 (s, 1H), 8.88 (d, | 455 | Method 94 |
| | Cyano-1-methyl | 2H), 8.58 (s, 1H), 8.20 (d, 1H), | | |
| | ethyl)-1-methyl- | 8.05 (d, 1H), 7.73 (d, 1H), 7.41 (dd, 1H), | | |
| | 1H-pyrazol-3-yl] | 7.05 (d, 1H), 6.63 (d, 1H), 3.92 (s, | | |
| | carbonyl}amino)- | 3H), 2.04 (s, 3H), 1.60 (s, 6H) | | |
| | 2-methylphenyl] | | | |
| | quinoxaline-6- | | | |
| | carboxamide | | | |
| 50 | N-{5-[(3- | 10.35 (s, 1H), 10.22 (s, 1H), | 424 | Method 92 |
| | Cyclopropyl | 9.07 (q, 2H), 8.77 (d, 1H), 8.37 (dd, 1H), | | |
| | benzoyl)amino]-2- | 8.25 (d, 1H), 7.88 (d, 1H), | | |
| | methylphenyl} | 7.71-7.60 (m, 3H), 7.29 (t, 1H), 7.28-7.25 (m, | | |
| | quinoxaline-6- | 2H), 2.25 (s, 3H), 1.19-1.16 (m, | | |
| | carboxamide | 1H), 1.00-0.97 (m, 2H), | | |
| | | 0.78-0.75 (m, 2H) | | |
| 51 | N-{5-[(3-tert-Butyl | 10.35 (s, 1H), 10.25 (s, 1H), | 440 | Method 124 |
| | benzoyl)amino]-2- | 9.06 (q, 2H), 8.77 (d, 1H), 8.37 (dd, 1H), | | |
| | methylphenyl} | 8.25 (d, 1H), 7.93 (s, 1H), 7.88 (d, | | |
| | quinoxaline-6- | 1H), 7.77 (d, 1H), 7.62-7.60 (m, | | |
| | carboxamide | 2H), 7.44 (t, 1H), 7.27 (d, 1H), | | |
| | | 2.26 (s, 3H), 1.33 (s, 9H) | | |
| 52 | N-(5-{[2-(1- | 10.60 (s, 1H), 10.36 (s, 1H), | 451 | Method 123 |
| | Cyano-1-methyl | 9.07 (q, 2H), 8.81 (d, 1H), 8.77 (d, 1H), | | |
| | ethyl) | 8.37 (dd, 1H), 8.23 (d, 1H), 8.01 (s, | | |
| | isonicotinoyl] | 1H), 7.88 (s, 2H), 7.62 (dd, 1H), | | |
| | amino}-2-methyl | 7.31 (d, 1H), 2.28 (s, 3H), 1.76 (s, | | |
| | phenyl)quinoxaline- | 6H) | | |
| | 6-carboxamide | | | |
| 53 | N-[5-({3-[(1- | 10.18 (s, 1H), 10.17 (s, 1H), 8.88 (s, | 492 | Method 87 |
| | Hydroxycyclopentyl) | 2H), 8.59 (s, 1H), 8.10 (q_{AB}, 2H), | | |
| | ethynyl] | 7.81-7.72 (m, 3H), 7.44-7.33 (m, | | |
| | benzoyl}amino)-2- | 3H), 7.10 (d, 1H), 5.19 (s, 1H), | | |
| | methylphenyl] | 2.07 (s, 3H), 1.80-1.70 (m, 4H), | | |
| | quinoxaline-6- | 1.69-1.49 (m, 4H) | | |
| | carboxamide | | | |
| 54 | N-(5-{[3-(3- | 10.35 (s, 2H), 9.06 (q, 2H), 8.77 (d, | 490 | Method 88 |
| | Cyclopentylprop- | 1H), 8.38 (dd, 1H), 8.25 (d, 1H), | | |
| | 1-yn-1-yl)benzoyl] | 7.97 (s, 1H), 7.90-7.87 (m, 2H), | | |
| | amino}-2-methyl | 7.63-7.47 (m, 3H), 7.28 (d, 1H), | | |
| | phenyl)quinoxaline- | 2.26 (s, 3H), 2.12-2.07 (m, 2H), | | |
| | 6-carboxamide | 1.80-1.77 (m, 2H), 1.65-1.50 (m, | | |
| | | 4H), 1.37-1.29 (m, 3H) | | |
| 55 | N-(5-{[3-(3,3- | 10.35 (s, 2H), 9.06 (q, 2H), 8.77 (d, | 463 | Method 102 |
| | Dimethylbut-1-yn- | 1H), 8.38 (dd, 1H), 8.25 (d, 1H), | | |
| | 1-yl)benzoyl] | 7.95 (s, 1H), 7.90-7.88 (m, 2H), | | |
| | amino}-2-methyl | 7.63 (dd, 1H), 7.60-7.46 (m, 2H), | | |
| | phenyl)quinoxaline- | 7.27 (d, 1H), 2.26 (s, 3H), 1.31 (s, | | |
| | 6-carboxamide | 9H) | | |
| 56 | N-[5-({3-[(2- | 10.36 (s, 1H), 10.33 (s, 1H), | 470 | Method 90 |
| | Methoxyethyl) | 9.05 (q, 2H), 8.72 (d, 1H), 8.39 (dd, 1H), | | |
| | (methyl)amino] | 8.24 (d, 1H), 7.90 (s, 1H), 7.88 (d, | | |
| | benzoyl}amino)-2- | 2H), 7.60 (dd, 1H), 7.59 (dd, 2H), | | |
| | methylphenyl] | 7.20 (d, 1H), 3.40 (d, 2H), 3.35 (s, | | |
| | quinoxaline-6- | 3H), 3.22-3.18 (m, 2H), 2.82 (s, | | |
| | carboxamide | 3H), 2.21 (s, 3H) | | |
| 57 | N-(5-{[3- | 10.36 (s, 1H), 10.33 (s, 1H), | 447 | Method 89 |
| | (Cyclopropyl | 9.07 (q, 2H), 8.78 (d, 1H), 8.39 (dd, 1H), | | |
| | ethynyl)benzoyl] | 8.29 (d, 1H), 7.97 (s, 1H), | | |
| | amino}-2-methyl | 7.89-7.85 (m, 2H), 7.62 (d, 1H), 7.58 (dd, | | |
| | phenyl)quinoxaline- | 2H), 7.26 (d, 1H), 2.28 (s, 3H), | | |
| | 6-carboxamide | 1.22-1.16 (m, 1H), 1.00-0.95 (m, | | |
| | | 2H), 0.81-0.74 (m, 2H) | | |
| 58 | N-(2-Methyl-5- | 10.35 (s, 1H), 10.34 (s, 1H), | 468 | Method 91 |
| | {[(5-piperidin-1-yl | 9.06 (q, 2H), 8.77 (d, 1H), 8.46-8.39 (m, | | |
| | pyridin-3-yl) | 2H), 8.37 (dd, 1H), 8.25 (d, 1H), | | |
| | carbonyl]amino} | 7.88 (d, 1H), 7.10 (t, 1H), 7.60 (dd, | | |
| | phenyl)quinoxaline- | 1H), 7.27 (d, 1H), 3.32-3.26 (m, | | |
| | 6-carboxamide | 4H), 2.26 (s, 3H), 1.70-1.58 (m, 6H) | | |
| 59 | N-{2-Methyl-5- | 10.39 (d, 2H), 9.07 (s, 2H), 8.77 (s, | 464 | 3-thien-2- |
| | [(3-thien-2-yl | 1H), 8.38 (d, 1H), 8.18-8.27 (m, | | ylbenzoic acid |
| | benzoyl)amino] | 2H), 7.83-7.94 (m, 3H), | | |
| | phenyl} | 7.54-7.66 (m, 4H), 7.29 (d, 1H), | | |
| | quinoxaline-6- | 7.14-7.22 (m, 1H), 2.27 (s, 3H) | | |
| | carboxamide | | | |
| 60 | N-{5-[(3-Hydroxy | 10.34 (s, 1H), 10.19 (s, 1H), 9.73 (s, | 398 | 3-hydroxybenzoic |
| | benzoyl)amino]-2- | 1H), 9.06 (d, 2H), 8.77 (s, 1H), | | acid |
| | methylphenyl} | 8.37 (d, 1H), 8.24 (d, 1H), 7.89 (s, 1H), | | |
| | quinoxaline-6- | 7.59 (d, 1H), 7.38 (d, 1H), | | |
| | carboxamide | 7.24-7.34 (m, 3H), 6.96 (d, 1H), 2.24 (s, | | |
| | | 3H) | | |
| 61 | N-[5-({3- | 10.40 (s, 1H), 10.34 (s, 1H), 9.07 (s, | 464 | 3- |
| | [(Difluoromethyl) | 2H), 8.77 (s, 1H), 8.38 (d, 1H), | | [(difluoromethyl) |
| | thio]benzoyl} | 8.24 (d, 1H), 8.17 (s, 1H), 8.07 (d, 1H), | | thio]benzoic acid |
| | amino)-2-methyl | 7.90 (s, 1H), 7.79 (d, 1H), | | |
| | phenyl]quinoxaline- | 7.43-7.71 (m, 3H), 7.29 (d, 1H), 2.27 (s, | | |
| | 6-carboxamide | 3H) | | |
| 62 | N-{5-[(3-Iodo | 10.34 (s, 2H), 9.02-9.11 (m, 2H), | 508 | 3-iodobenzoic |
| | benzoyl)amino]-2- | 8.77 (s, 1H), 8.38 (d, 1H), 8.30 (s, | | acid |
| | methylphenyl} | 1H), 8.24 (d, 1H), 7.95 (t, 2H), | | |
| | quinoxaline-6- | 7.88 (s, 1H), 7.61 (d, 1H), 7.26-7.36 (m, | | |
| | carboxamide | 2H), 2.26 (s, 3H) | | |
| 63 | N-[5-({3-[(3,5- | 10.35 (d, 2H), 9.06 (s, 2H), 8.77 (s, | 490 | 3-[(3,5-dimethyl- |
| | Dimethyl-1H- | 1H), 8.38 (d, 1H), 8.24 (d, 1H), | | 1H-pyrazol-1-yl) |
| | pyrazol-1-yl) | 7.89 (s, 2H), 7.75 (s, 1H), 7.61 (d, 1H), | | methyl]benzoic |
| | methyl]benzoyl} | 7.48 (t, 1H), 7.27 (d, 2H), 5.95 (s, | | acid |
| | amino)-2-methyl | 1H), 5.32 (s, 2H), 2.26 (s, 3H), | | |
| | phenyl]quinoxaline- | 2.21 (s, 3H), 2.13 (s, 3H) | | |
| | 6-carboxamide | | | |
| 64 | N-{2-Methyl-5- | 10.56 (s, 1H), 10.36 (s, 1H), 9.07 (s, | 468 | 2-morpholin-4- |
| | [(2-morpholin-4- | 2H), 8.78 (s, 1H), 8.38 (d, 1H), | | ylisonicotinic acid |
| | ylisonicotinoyl) | 8.21-8.30 (m, 2H), 7.89 (s, 1H), 7.63 (d, | | |
| | amino]phenyl} | 1H), 7.51 (s, 1H), 7.30 (d, 1H), | | |
| | quinoxaline-6- | 7.20 (d, 1H), 3.74 (d, 4H), 3.64 (d, 4H), | | |
| | carboxamide | 2.27 (s, 3H) | | |
| 65 | N-[5-({3-[(2,2- | 10.35 (s, 1H), 10.27 (s, 1H), 9.41 (s, | 481 | 3-[(2,2-dimethyl |
| | Dimethyl | 1H), 9.04-9.08 (m, 2H), 8.77 (s, | | propanoyl)amino] |
| | propanoyl)amino] | 1H), 8.38 (d, 1H), 8.24 (d, 1H), | | benzoic acid |
| | benzoyl}amino)-2- | 8.15 (s, 1H), 7.86-7.92 (m, 2H), 7.62 (t, | | |
| | methylphenyl] | 2H), 7.43 (t, 1H), 7.27 (d, 1H), | | |
| | quinoxaline-6- | 2.25 (s, 3H), 1.23 (s, 9H) | | |
| | carboxamide | | | |
| 66 | N-{5-[(3-Butoxy | 10.34 (s, 1H), 10.22 (s, 1H), | 454 | 3-butoxybenzoic |
| | benzoyl)amino]-2- | 9.06 (d, 2H), 8.77 (s, 1H), 8.38 (d, 1H), | | acid |
| | methylphenyl} | 8.24 (d, 1H), 7.89 (s, 1H), 7.62 (d, | | |
| | quinoxaline-6- | 1H), 7.48-7.54 (m, 2H), 7.42 (t, | | |
| | carboxamide | 1H), 7.27 (d, 1H), 7.14 (d, 1H), | | |
| | | 4.04 (t, 2H), 2.26 (s, 3H), 1.67-1.76 (m, | | |
| | | 2H), 1.45 (qt, 2H), 0.94 (t, 3H) | | |
| 67 | N-[5-({[2,6-Bis | 10.64 (s, 1H), 10.38 (s, 1H), 9.07 (s, | 470 | 2,6-bis |
| | (dimethylamino) | 2H), 8.78 (s, 1H), 8.38 (d, 1H), | | (dimethylamino) |
| | pyrimidin-4-yl] | 8.24 (d, 1H), 7.92 (s, 1H), 7.68 (d, 1H), | | pyrimidine-4- |
| | carbonyl}amino)- | 7.31 (d, 1H), 6.89 (s, 1H), 3.18 (s, | | carboxylic acid |
| | 2-methylphenyl] | 12H), 2.27 (s, 3H) | | |
| | quinoxaline-6- | | | |
| | carboxamide | | | |
| 68 | N-(5-{[(2,6- | 10.38 (s, 1H), 10.23 (s, 1H), 9.07 (s, | 554 | 2,6-dimorpholin- |
| | Dimorpholin-4- | 2H), 8.78 (s, 1H), 8.37 (d, 1H), | | 4-ylpyrimidine-4- |
| | ylpyrimidin-4- | 8.24 (d, 1H), 7.89 (s, 1H), 7.70 (d, 1H), | | carboxylic acid |
| | yl)carbonyl] | 7.29 (d, 1H), 6.78 (s, 1H), | | |
| | amino}-2-methyl | 3.63-3.76 (m, 16H), 2.26 (s, 3H) | | |
| | phenyl)quinoxaline- | | | |
| | 6-carboxamide | | | |
| 69 | N-(5-{[3,5-Bis | 10.70 (s, 1H), 10.36 (s, 1H), 9.07 (s, | 518 | 3,5-bis |
| | (trifluoromethyl) | 2H), 8.78 (s, 1H), 8.62 (s, 2H), | | (trifluoromethyl) |
| | benzoyl]amino}-2- | 8.37 (d, 2H), 8.25 (d, 1H), 7.89 (s, 1H), | | benzoic acid |
| | methylphenyl) | 7.66 (d, 1H), 7.32 (d, 1H), 2.28 (s, | | |
| | quinoxaline-6- | 3H) | | |
| | carboxamide | | | |
| 70 | N-(5-{[3-(1- | 8.92-9.00 (m, 2H), 8.70 (s, 1H), | 503 | Method 98 |
| | Cyano-1- | 8.34 (d, 1H), 8.21 (d, 1H), 7.99 (s, | | |
| | methylethyl)-5-(3- | 1H), 7.92 (s, 1H), 7.81 (s, 1H), | | |
| | hydroxyprop-1-yn- | 7.75 (s, 1H), 7.55 (d, 1H), 7.29 (d, 1H), | | |
| | 1-yl)benzoyl] | 4.39 (s, 2H), 2.31 (s, 3H), 1.74 (s, | | |
| | amino}-2-methyl | 6H) | | |
| | phenyl)quinoxaline- | | | |
| | 6-carboxamide | | | |
| 71 | N-[5-({3-(1- | 8.95-9.02 (m, 2H), 8.73 (s, 1H), | 585 | Method 100 |
| | Cyano-1-methyl | 8.37 (d, 1H), 8.23 (d, 1H), 8.04 (s, | | |
| | ethyl)-5-[3-(4- | 1H), 7.97 (s, 1H), 7.85 (s, 1H), | | |
| | methylpiperazin-1- | 7.78 (s, 1H), 7.58 (d, 1H), 7.31 (d, 1H), | | |
| | yl)prop-1-yn-1- | 3.68 (s, 2H), 3.00 (s, 4H), 2.85 (s, | | |
| | yl]benzoyl}amino)- | 4H), 2.63 (s, 3H), 2.33 (s, 3H), | | |
| | 2-methylphenyl] | 1.77 (s, 6H) | | |
| | quinoxaline-6- | | | |
| | carboxamide | | | |
| 72 | N-(5-{[3-(1- | 8.97-9.05 (m, 2H), 8.75 (s, 1H), | 491 | Method 99 |
| | Cyano-1-methyl | 8.40 (d, 1H), 8.25 (d, 1H), | | |
| | ethyl)-5-propyl | 7.83-7.93 (m, 2H), 7.75 (s, 1H), 7.55-7.62 (m, | | |
| | benzoyl]amino}-2- | 2H), 7.33 (d, 1H), 2.69-2.79 (m, | | |
| | methylphenyl) | 2H), 2.36 (s, 3H), 1.68-1.82 (m, | | |
| | quinoxaline-6- | 8H), 0.99 (t, 3H) | | |
| | carboxamide | | | |
| 73 | N-[5-({3- | 10.56 (s, 1H), 10.35 (s, 1H), | 490 | Method 128 |
| | [(Dimethylamino) | 9.07 (d, 2H), 8.78 (s, 1H), 8.38 (d, 1H), | | |
| | sulfonyl]benzoyl} | 8.23-8.34 (m, 3H), 7.87-7.98 (m, | | |
| | amino)-2-methyl | 2H), 7.82 (t, 1H), 7.64 (d, 1H), | | |
| | phenyl]quinoxaline- | 7.30 (d, 1H), 2.65 (s, 6H), 2.27 (s, 3H) | | |
| | 6-carboxamide | | | |
| 74 | N-(2-Methyl-5- | 10.57 (s, 1H), 10.36 (s, 1H), 9.07 (s, | 461 | 3-(methyl |
| | {[3-(methyl | 2H), 8.77 (s, 1H), 8.48 (s, 1H), | | sulfonyl)benzoic |
| | sulfonyl)benzoyl] | 8.35-8.40 (m, 1H), 8.23-8.32 (m, 2H), | | acid |
| | amino}phenyl) | 8.13 (d, 1H), 7.89 (s, 1H), 7.82 (t, | | |
| | quinoxaline-6- | 1H), 7.63 (d, 1H), 7.30 (d, 1H), | | |
| | carboxamide | 3.29 (s, 3H), 2.27 (s, 3H) | | |
| 75 | N-(5-{[3-(1- | 10.35 (s, br, 1H), 10.20 (s, 1H), | 562 | Method 101 |
| | Cyano-1-methyl | 10.16 (s, 1H), 8.86 (s, 2H), 8.60 (s, | | |
| | ethyl)-5-(2- | 1H), 8.20 (d, 1H), 8.05 (d, 1H), | | |
| | pyrrolidin-1- | 7.65 (s, 1H), 7.50 (s, 1H), 7.45 (d, 1H), | | |
| | ylethoxy)benzoyl] | 7.37 (s, 1H), 7.10 (m, 2H), 4.25 (t, | | |
| | amino}-2-methyl | 2H), 3.42 (m, 2h), 2.99 (m, 4H), | | |
| | phenyl)quinoxaline- | 2.10 (s, j3H), 1.88 (m, 2H), 1.73 (m, | | |
| | 6-carboxamide | 2H), 1.60 (s, 6H) | | |

### Example 76

### N-(5-{[3-(1-Cyano-1-methylethyl)benzoyl]amino}-2-fluorophenyl)quinoxaline-6-carboxamide

A solution of quinoxaline-6-carboxylic acid (141 mg, 0.81 mmol) in thionyl chloride (3 ml) was heated at 80° C. for 1 h. The volatile components were removed under reduced pressure. To the resultant residue in DMF (3 ml) was added DIEA (0.28 ml, 1.60 mmol) and N-(3-amino-4-fluorophenyl)-3-(1-cyano-1-methylethyl)benzamide (Method 57; 118 mg, 0.40 mmol) in DMF (1 ml) and the reaction mixture was allowed to stir at 25° C. for 30 min. The reaction mixture was partitioned between EtOAc and H₂O and the organics were washed with H₂O and dried (MgSO₄(s)). The solvent was removed under reduced pressure and product was purified by preparative HPLC to give 75.0 mg of the title compound. (42.0%) NMR (300 MHz): 10.63 (s, 1H), 10.47 (s, 1H), 9.08 (s, 2H), 8.79 (s, 1H), 8.43 (d, 1H), 8.27 (d, 1H), 7.88-8.19 (m, 3H), 7.50-7.86 (m, 3H), 7.36 (t, 1H), 1.76 (s, 6H); m/z 454.

### Examples 77-80

The following compounds were prepared by the procedure of Example 76 using the appropriate SM and quinoxaline-6-carboxylic acid.

| Ex. | Compound | NMR | m/z | SM |
|---|---|---|---|---|
| 77 | N-(2-Bromo-5-{[3-(1- | 10.56 (s, 2H), 9.09 (s, 2H), 8.81 (s, | 515 | Method 58 |
| | cyano-1-methylethyl) | 1H), 8.41 (d, 1H), 8.27 (d, 1H), | | |
| | benzoyl]amino}phenyl) | 8.02-8.16 (m, 2H), 7.96 (d, 1H), | | |
| | quinoxaline-6- | 7.70-7.87 (m, 3H), 7.63 (t, 1H), | | |
| | carboxamide | 1.76 (s, 6H) | | |
| 78 | N-(3-Bromo-5-{[3-(1- | 10.75 (s, 1H), 10.47 (s, 1H), | 529 | Method 59 |
| | cyano-1-methylethyl) | 9.02-9.25 (m, 2H), 8.82 (s, 1H), 8.40 (d, | | |
| | benzoyl]amino}-2- | 1H), 8.15-8.33 (m, 2H), 8.05 (s, | | |
| | methylphenyl)quinoxaline- | 1H), 7.94 (s, 1H), 7.87 (d, 1H), | | |
| | 6-carboxamide | 7.43 (t, 1H), 7.28 (d, 1H), 2.42 (s, | | |
| | | 3H), 1.53-1.84 (m, 6H) | | |
| 79 | N-(2-Chloro-5-{[3-(1- | 10.55 (s, 1H), 9.12 (s, 2H), 8.81 (s, | 470 | Method 63 |
| | cyano-1-methylethyl) | 1H), 8.39 (d, 1H), 8.27 (d, 1H), | | |
| | benzoyl]amino} | 8.14 (s, 1H), 8.05 (s, 1H), 7.97 (d, | | |
| | phenyl)quinoxaline-6- | 1H), 7.74-7.85 (m, 2H), | | |
| | carboxamide | 7.52-7.67 (m, 2H), 1.75 (s, 6H) | | |
| 80 | N-(5-{[3-(1-Cyano-1- | 10.33 (s, 1H), 10.01 (s, 1H), | 566 | Method 64 |
| | methylethyl)benzoyl] | 8.95-9.20 (m, 2H), 8.75 (s, 1H), 8.37 (d, | | |
| | amino}-2-methoxyphenyl) | 1H), 8.14-8.33 (m, 2H), 8.08 (s, | | |
| | quinoxaline-6- | 1H), 7.97 (d, 1H), 7.66-7.83 (m, | | |
| | carboxamide | 2H), 7.61 (t, 1H), 7.15 (d, 1H), | | |
| | | 3.87 (s, 3H), 1.75 (s, 6H) | | |

### Example 81

### 3-(1-Cyano-1-methylethyl)-N-[4-methyl-3-(quinoxalin-6-ylamino)phenyl]benzamide

N-(3-Amino-4-methylphenyl)-3-(1 cyano-1-methylethyl)benzamide (Method 60; 0.150 g, 0.51 mmol), 6-bromoquinoxaline (0.109 g, 0.51 mmol), Pd₂(dba)₃ (0.024 g, 0.026 mmol), BINAP (0.032 g, 0.051 mmol), and sodium tert-butoxide (0.147 g, 1.53 mmol) were combined in toluene (3 ml) in a sealed tube under an argon atmosphere and heated to 100° C. for 15 hours. The reaction mixture was filtered over diatomaceous earth, concentrated and purified by reverse phase preparative HPLC. NMR (300 MHz): 10.24 (s, 1H), 8.62 (d, 1H), 8.51 (d, 1H), 8.31 (s, 1H), 7.94 (t, 1H), 7.77-7.90 (m, 3H), 7.62-7.72 (m, 1H), 7.48-7.58 (m, 2H), 7.45 (dd, 1.98, 1H), 7.23 (d, 1H), 7.02 (d, 1H), 2.16 (s, 3H), 1.67 (s, 6H); m/z 422.

### Example 82

### N-(2-Methyl-5-{[3-(trifluoromethyl)benzyl]amino}phenyl)quinoxaline-6-carboxamide

N-(5-Amino-2-methylphenyl)quinoxaline-6-carboxamide hydrochloride (Method 4; 0.080 g, 0.253 mmol), 3-(trifluoromethyl)benzaldehyde (0.044 g, 0.253 mmol) and sodium triacetoxyborohydride (0.059 g, 0.278 mmol) were combined in 1,2-dichloroethane (4 ml) and allowed to stir for 5 h at 25° C. The reaction mixture was concentrated under reduced pressure and purified by reverse phase preparative HPLC. NMR (300 Mz): 10.05 (s, 1H), 8.94-9.05 (m, 2H), 8.65 (s, 1H), 8.27 (dd, 1H), 8.10-8.22 (m, 1H), 7.68 (s, 1H), 7.58-7.65 (m, 1H), 7.45-7.57 (m, 2H), 6.94 (d, 1H), 6.71 (s, 1H), 6.44 (dd, 1H), 4.33 (s, 2H), 2.05 (s, 3H); m/z 437.

### Example 83

The following compound was prepared by the procedure of Example 82 using the appropriate SM and N-(3-amino-4-methylphenyl)-3-(trifluoromethyl)benzamide hydrochloride (Method 65).

| Ex. | Compound | NMR | m/z | SM |
|---|---|---|---|---|
| 83 | N-{4-Methyl-3- | 10.03 (s, 1H), 8.78-8.85 (m, 2H), | 437 | quinoxaline-6- |
| | [(quinoxalin-6-yl | 8.03-8.09 (m, 2H), 8.01 (d, 2H), | | carbaldehyde |
| | methyl)amino]phenyl}- | 7.90-7.95 (m, 1H), 7.84 (dd, 2H), | | |
| | 3-(trifluoromethyl) | 7.64 (t, 1H), 6.92 (s, 2H), 6.87 (s, | | |
| | benzamide | 1H), 4.56 (s, 2H), 2.14 (s, 3H) | | |

### Example 84

### N-(5-{[(1-tert-Butyl-3-methyl-1H-pyrazol-5-yl)carbonyl]amino}-2-methylphenyl)quinoxaline-6-carboxamide

N-(5-Amino-2-methylphenyl)quinoxaline-6-carboxamide hydrochloride (Method 4; 0.080 g, 0.253 mmol), 1-tert-butyl-3-methyl-1H-pyrazole-5-carbonyl chloride (0.071, 0.351 mmol) and triethylamine (0.115 ml, 0.759 mmol) were combined in 4 ml anhydrous DCM and allowed to stir for 1 hour at 25° C. The reaction mixture was concentrated under reduced pressure and purified by reverse phase preparative HPLC. NMR (300 MHz): 10.28 (s, 1H), 9.57 (s, 1H), 9.01 (d, 2H), 8.71 (s, 1H), 8.24-8.36 (m, 1H), 8.18 (d, 1H), 7.81 (s, 1H), 7.58 (dd, 1H), 7.19 (d, 1H), 6.51 (s, 1H), 2.41 (s, 3H), 2.18 (s, 3H), 1.57 (s, 9H); m/z 443.

### Example 85

### 2,3-Dimethyl-N-(2-methyl-5-{[3-(trifluoromethyl)benzoyl]amino}phenyl)quinoxaline-6-carboxamide

A solution of N-(3-amino-4-methylphenyl)-3-(trifluoromethyl)benzamide hydrochloride (Method 65; 0.080 g, 0.27 mmol), 2,3-dimethylquinoxaline-6-carboxylic acid (0.055 g, 0.27 mmol) and diisopropylethylamine (141 μl, 0.81 mmol) in 2 ml of DMF was treated with HATU (0.123 g, 0.32 mmol). The reaction mixture was allowed to stir at 50° C. for 15 hours. The reaction was quenched with H₂O and extracted with EtOAc. The organics were dried with NaCl (sat) and then Na₂SO₄ (s) and removed under reduced pressure. The resulting solid was purified by reverse phase preparative chromatography. NMR (300 MHz): 10.45 (s, 1H), 10.16 (s, 1H), 8.58 (d, 1H), 8.14-8.28 (m, 3H), 8.02 (d, 1H), 7.91 (d, 1H), 7.84 (d, 1H), 7.73 (t, 1H), 7.58 (dd, 1H), 7.23 (d, 1H), 2.67 (s, 6H), 2.21 (8, 3H); m/z 479.

### Example 86

The following compounds were prepared by the procedure of Example 85 using the appropriate SM and Method 65.

| Ex. | Compound | NMR | m/z | SM |
|---|---|---|---|---|
| 86 | N-(2-Methyl-5-{[3- | 10.45 (s, 1H), 10.29 (s, 1H), | 451 | quinoxaline- |
| | (trifluoromethyl) | 8.95-9.07 (m, 2H), 8.72 (d, 1H), 8.32 (dd, 1H), | | 6-carboxylic |
| | benzoyl]amino} | 8.24-8.28 (m, 1H), 8.16-8.24 (m, 2H), | | acid |
| | phenyl)quinoxaline- | 7.90 (d, 1H), 7.84 (d, 1H), 7.73 (t, 1H), | | |
| | 6-carboxamide | 7.58 (dd, 1H), 7.24 (d, 1H), 2.22 (s, 3H) | | |

### Example 87

### N-{2-Methyl-5-[(3-nitrobenzyl)amino]phenyl}quinoxaline-6-carboxamide

To a solution of 50 mg (0.18 mmol) of N-(5-amino-2-methylphenyl)quinoxaline-6-carboxamide hydrochloride (Method 4; 50 mg, 0.18 mmol) and triethylamine (35 μl) in DMF (2 ml) was added 1-(bromomethyl)-3-nitrobenzene (54 mg, 0.25 mmol) and the mixture was shaken at 60° C. for 3 h. The reaction mixture was poured onto H₂O (20 ml) and the resultant solids were collected by filtration and washed with water. The solid was chromatographed on silica gel to give 8 mg of the title compound. NMR: 10.09 (s, 1H), 9.06 (s, 2H), 8.71 (s, 1H), 8.32 (s, 1H), 8.22 (s, 2H), 8.09 (s, 1H), 7.84 (s, 1H), 7.63 (s, 1H), 6.96 (s, 1H), 6.69 (s, 1H), 6.42 (s, 2H), 4.42 (s, 2H), 3.31 (s, 2H), 2.08 (s, 3H); m/z 414.

### Example 88

### N-(5-{[3-Amino-5-(1-cyano-1-methylethyl)benzoyl]amino}-2-methylphenyl)quinoxaline-6-carboxamide

tert-Butyl {3-(1-cyano-1-methylethyl)-5-[({4-methyl-3-[(quinoxalin-6-ylcarbonyl)amino]phenyl}amino)carbonyl]phenyl}carbamate (Example 44; 314 mg, 0.556 mmol) in 4 N HCl in dioxane (14 ml) was stirred for 2 hours. The solvent was removed under reduced pressure and the brown crude was purified on reverse phase preparative HPLC to give 36 mg (14%) of the title compound as white solid. NMR (300 MHz): 10.28 (s, 1H), 10.08 (s, 1H), 9.07-8.93 (m, 2H), 8.71 (s, 1H), 8.39-8.24 (m, 1H), 8.19 (d, 1H), 7.78 (s, 1H), 7.53 (d, 1H), 7.21 (d, 1H), 7.09-7.03 (m, 1H), 7.02-6.94 (m, 1H), 6.90-6.78 (m, 1H), 5.47 (s, 2H), 2.20 (s, 3H), 1.62 (s, 6H); m/z 464.

### Example 89

### 2-Chloro-N-(5-{[3-(1-cyano-1-methylethyl)benzoyl]amino}-2-methylphenyl)quinoxaline-6-carboxamide

N-(3-Amino-4-methylphenyl)-3-(1-cyano-1-methylethyl)benzamide (Method 60; 0.694 g, 2.37 mmol) was added to 2-chloroquinoxaline-6-carbonyl chloride (Method 106; 0.537 g, 2.37 mmol) and triethylamine (1.65 ml, 11.85 mmol) in 30 ml DCM and stirred for 1 h at 25° C. The solvents were removed under reduced pressure and the resultant product was used without further purification; m/z 484.

### Example 90

### N-(5-{[3-(1-Cyano-1-methylethyl)benzoyl]amino}-2-methylphenyl)-2-[(3-piperidin-1-ylpropyl)amino]quinoxaline-6-carboxamide

3-Piperidin-1-ylpropan-1-amine (1 ml) was added to a stirring solution of 2-chloro-N-(5-{[3-(1-cyano-1-methylethyl)benzoyl]amino}-2-methylphenyl)quinoxaline-6-carboxamide (Example 89; 0.060 g, 0.123 mmol) in MeOH (3 ml) and the reaction mixture was stirred for 2 h at 60° C. The solvent was removed under reduced pressure and product was purified by reverse phase semi-preparative HPLC. NMR (300 MHz): 10.35 (s, 1H), 10.06 (s, 1H), 8.49 (s, 1H), 8.40 (s, 1H), 8.01-8.20 (, 2H), 7.80-7.99 (m, 2H), 1.71-7.79 (m, 1H), 7.52-7.70 (m, 2H), 7.21-7.36 (m, 2H), 7.14 (s, 1H), 3.33-3.61 (m, 4H), 2.99-3.25 (m, 2H), 2.77-2.96 (m, 2H), 2.25 (s, 3H), 1.96-2.10 (m, 2H), 1.76 (s, 6H), 1.58-1.89 (m, 4H), 1.28-1.53 (m, 2H); m/z 590.

### Examples 91-99

The following compounds were prepared by the procedure of Example 90 using the appropriate SM and 2-chloro-N-(5-{[3-(1-cyano-1-methylethyl)benzoyl]amino}-2-methylphenyl)quinoxaline-6-carboxamide (Example 89).

| Ex. | Compound | NMR | m/z | SM |
|---|---|---|---|---|
| 91 | N-(5-{[3-(1-Cyano-1- | 10.29 (s, 1H), 9.99 (s, 1H), | 535 | Morpholine |
| | methylethyl)benzoyl] | 8.87 (s, 1H), 8.48 (s, 1H), 8.10 (d, | | |
| | amino}-2-methyl | 1H), 7.99 (s, 1H), 7.88 (d, 1H), | | |
| | phenyl)-2-morpholin- | 7.78 (s, 1H), 7.59-7.73 (m, 2H), | | |
| | 4-ylquinoxaline-6- | 7.48-7.59 (m, 2H), 7.21 (d, | | |
| | carboxamide | 1H), 3.73-3.80 (m, 4H), | | |
| | | 3.66-3.73 (m, 4H), 2.19 (s, 3H), | | |
| | | 1.69 (s, 6H) | | |
| 92 | 2-[(3-Amino | 10.29 (s, 1H), 9.99 (s, 1H), | 522 | Propane-1,3- |
| | propyl)amino]-N-(5- | 8.42 (s, 1H), 8.36 (s, 1H), 8.08 (d, | | diamine |
| | {[3-(1-cyano-1- | 1H), 7.97-8.01 (m, 1H), | | |
| | methylethyl)benzoyl] | 7.88 (d, 1H), 7.75-7.83 (m, 2H), | | |
| | amino}-2-methyl | 7.69 (d, 1H), 7.60 (d, 1H), | | |
| | phenyl)quinoxaline-6- | 7.47-7.57 (m, 2H), 7.21 (d, 1H), | | |
| | carboxamide | 3.55-3.70 (m, 2H), 2.79-2.91 (m, | | |
| | | 2H), 2.18 (s, 3H), 1.79-1.93 (m, | | |
| | | 2H), 1.69 (s, 6H) | | |
| 93 | N-(5-{[3-(1-Cyano-1- | 10.27 (s, 1H), 9.99 (s, 1H), | 507 | Ethyl(methyl)amine |
| | methylethyl)benzoyl] | 8.72 (s, 1H), 8.45 (s, 1H), 8.08 (d, | | |
| | amino}-2-methyl | 1H), 7.99 (s, 1H), 7.88 (d, 1H), | | |
| | phenyl)-2-[ethyl | 7.78 (s, 1H), 7.64-7.71 (m, 1H), | | |
| | (methyl)amino] | 7.48-7.63 (m, 3H), 7.21 (d, | | |
| | quinoxaline-6- | 1H), 3.71 (q, 2H), 3.17 (s, 3H), | | |
| | carboxamide | 2.19 (s, 3H), 1.69 (s, 6H), | | |
| | | 1.14 (t, 3H) | | |
| 94 | N-(5-{[3-(1-Cyano-1- | 10.28 (s, 1H), 9.99 (s, 1H), | 479 | Methylamine |
| | methylethyl)benzoyl] | 8.39 (s, 1H), 8.30 (s, 1H), 8.05 (dd, | | |
| | amino}-2-methyl | 1H), 7.98 (s, 1H), 7.88 (d, 1H), | | |
| | phenyl)-2-(methyl | 7.77 (s, 1H), 7.68 (dd, 1H), | | |
| | amino)quinoxaline-6- | 7.51-7.61 (m, 2H), 7.14-7.26 (m, | | |
| | carboxamide | 2H), 7.01 (s, 1H), 2.85-2.92 (m, | | |
| | | 1H), 2.18 (s, 3H), 1.69 (s, 6H) | | |
| 95 | N-(5-{[3-(1-Cyano-1- | 10.30 (s, 1H), 10.02 (s, 1H), | 536 | N,N- |
| | methylethyl)benzoyl] | 8.45 (s, 1H), 8.37 (s, 1H), | | Dimethylethane- |
| | amino}-2-methyl | 8.27-8.33 (m, 1H), 8.07-8.13 (m, 1H), | | 1,2-diamine |
| | phenyl)-2-{[2- | 7.99 (d, 1H), 7.85-7.93 (m, | | |
| | (dimethylamino)ethyl] | 1H), 7.80 (s, 1H), 7.59-7.71 (m, | | |
| | amino}quinoxaline-6- | 2H), 7.48-7.58 (m, 2H), | | |
| | carboxamide | 7.21 (d, 1H), 3.68-3.80 (m, 2H), | | |
| | | 3.24-3.33 (m, 2H), | | |
| | | 2.77-2.83 (m, 6H), 2.18 (s, 3H), 1.69 (s, | | |
| | | 6H) | | |
| 96 | N-(5-{[3-(1-Cyano-1- | 10.26 (s, 1H), 9.96 (s, 1H), | 509 | 2-Aminoethanol |
| | methylethyl)benzoyl] | 8.33-8.42 (m, 2H), 8.05 (dd, 1H), | | |
| | amino}-2-methyl | 7.98 (s, 1H), 7.88 (d, 1H), | | |
| | phenyl)-2-[(2- | 7.77 (d, 1H), 7.63-7.71 (m, 1H), | | |
| | hydroxyethyl)amino] | 7.52-7.58 (m, 2H), | | |
| | quinoxaline-6- | 7.16-7.25 (m, 2H), 7.03 (s, 1H), | | |
| | carboxamide | 3.53-3.61 (m, 2H), 3.40-3.51 (m, 2H), | | |
| | | 2.18 (s, 3H), 1.69 (s, 6H) | | |
| 97 | N-(5-{[3-(1-Cyano-1- | 10.28 (s, 1H), 10.03 (s, 1H), | 536 | N,N′- |
| | methylethyl)benzoyl] | 8.75 (s, 1H), 8.50 (s, 1H), 8.12 (d, | | Dimethylethane- |
| | amino}-2-methyl | 1H), 7.98 (s, 1H), 7.88 (d, 1H), | | 1,2-diamine |
| | phenyl)-2-{methyl[2- | 7.80 (s, 1H), 7.61-7.73 (m, 2H), | | |
| | (methylamino)ethyl] | 7.48-7.58 (m, 2H), 7.21 (d, | | |
| | amino}quinoxaline-6- | 1H), 3.87-4.01 (m, 2H), | | |
| | carboxamide | 3.15-3.24 (m, 5H), 2.56 (s, 3H), | | |
| | | 2.19 (s, 3H), 1.69 (s, 6H) | | |
| 98 | N-(5-{[3-(1-Cyano-1- | 10.29 (s, 1H), 10.02 (s, 1H), | 578 | (2-Morpholin-4- |
| | methylethyl)benzoyl] | 8.46 (s, 1H), 8.38 (s, 1H), | | ylethyl)amine |
| | amino}-2-methyl | 8.23-8.29 (m, 1H), 8.07-8.14 (m, 1H), | | |
| | phenyl)-2-[(2- | 7.99 (s, 1H), 7.86-7.92 (m, 1H), | | |
| | morpholin-4-ylethyl) | 7.80 (s, 1H), 7.59-7.72 (m, 2H), | | |
| | amino]quinoxaline-6- | 7.49-7.59 (m, 2H), | | |
| | carboxamide | 3.86-4.00 (m, 4H), 3.73-3.84 (m, 4H), | | |
| | | 3.45-3.58 (m, 2H), | | |
| | | 3.06-3.21 (m, 2H), 2.19 (s, 3H), 1.69 (s, | | |
| | | 6H) | | |
| 99 | N-(5-{[3-(1-Cyano-1- | 10.30 (s, 1H), 10.02 (s, 1H), | 564 | N,N,N′- |
| | methylethyl)benzoyl] | 8.76 (s, 1H), 8.48 (s, 1H), 8.10 (d, | | Trimethylpropane- |
| | amino}-2-methyl | 1H), 7.96-8.03 (m, 1H), | | 1,3-diamine |
| | phenyl)-2-[[3- | 7.85-7.92 (m, 1H), 7.80 (s, 1H), | | |
| | (dimethylamino) | 7.68 (d, 1H), 7.62 (d, 1H), | | |
| | propyl](methyl) | 7.49-7.57 (m, 2H), 7.21 (d, 1H), | | |
| | amino]quinoxaline-6- | 3.68-3.80 (m, 2H), 3.21 (s, 3H), | | |
| | carboxamide | 2.98-3.13 (m, 2H), 2.68-2.73 (m, | | |
| | | 6H), 2.19 (s, 3H), 1.91-2.07 (m, | | |
| | | 2H), 1.69 (s, 6H) | | |

### Preparation of Starting Materials

### Method 1

### tert-Butyl (4-methyl-3-nitrophenyl)carbamate

To a mixture of potassium carbonate (172.33 g, 1.25 mol) in water (700 ml) and THF (700 ml) at 0° C. was added a solution of 4-methyl-3-nitroaniline (63.25 g, 0.41 mol) in THF (700 ml) followed by the addition of di-tert-butyl dicarbonate (99.78 g, 0.46 mol) in THF (700 ml). The reaction mixture was then stirred under nitrogen and allowed to warm to 25° C. over 15 h. The solvent was removed under reduced pressure and the crude residue was purified by column chromatography; m/z 251 [M-H]⁻.

### Method 2

### tert-Butyl (3-amino-4-methylphenyl)carbamate

tert-Butyl (4-methyl-3-nitrophenyl)carbamate (Method 1; 31.54 g, 0.125 mol) and 10% Pd/C (1.71 g, 1.6 mmol) in methanol (200 ml) were shaken under 45 psi hydrogen for 90 min. The reaction mixture was filtered through diatomaceous earth and concentrated under reduced pressure giving 26.62 g of the title product (96%); NMR (300 MHz): 8.93 (s, 1H), 6.83 (s, 1H), 6.73 (d, 1H), 6.47 (dd, 1H), 4.74 (s, 1H), 1.95 (s, 3H), 1.45 (s, 9H).

### Method 3

### tert-Butyl {4-methyl-3-[(quinoxalin-6-ylcarbonyl)amino]phenyl}carbamate

A solution of tert-butyl (3-amino-4-methylphenyl)carbamate (Method 2; 50.10 g, 0.23 mol), quinoxaline-6-carboxylic acid (50.10 g, 0.23 mol) and diisopropylethylamine (70 ml, 0.68 mol) in DMF (575 ml) was treated with HATU (94.3 g, 0.25 mol). The reaction was stirred at 25° C. for 24 h. The reaction was quenched with H₂O and extracted with EtOAc. The organics were dried with NaCl (sat) and then Na₂SO₄ (s) and removed under reduced pressure. The resulting solid was recrystallized from DCM/hexanes affording the product as brown crystals; m/z 379.

### Method 4

### N-(5-Amino-2-methylphenyl)quinoxaline-6-carboxamide hydrochloride

To tert-butyl {4-methyl-3-[(quinoxalin-6-ylcarbonyl)amino]phenyl}carbamate (Method 3; 107.76 g, 0.29 mol) was added 4 M HCl in dioxane. The reaction was stirred at 25° C. for 24 h. Twice the volume of diethyl ether was added resulting in precipitation of the product which was collected by vacuum filtration to give 72.1 g (79%); m/z 279.

### Method 5

### Methyl 4-fluoro-3-methylbenzoate

To a stirring solution of 4-fluoro-3-methylbenzoic acid (5.0 g, 0.032 mol) and potassium carbonate (9.0 g 0.064 mol) in DMF (80 ml) was added iodomethane (2.4 ml, 0.038 mol). The reaction mixture was allowed to stir at 25° C. for 15 h. The DMF was removed under reduced pressure and the resulting residue was washed with EtOAc and H₂O. The organic layer was dried and the solvent was removed under reduced pressure; m/z 169.

### Method 6

### Methyl 3-(bromomethyl)-4-chlorobenzoate

A solution of methyl 4-chloro-3-methylbenzoate (2.50 g, 13.54 mmol) and N-bromosuccinimide (3.00 g, 16.93 mmol) in carbon tetrachloride (50 ml) was treated with azobisisobutyronitrile (500 mg). The solution was heated to 80° C. for 4 h. before being cooled to room temperature. The reaction mixture was filtered through diatomaceous earth and the filtrate was concentrated under reduced pressure. The product was purified by column chromatography utilizing an ISCO system (hexanes/EtOAc) giving 2.70 g of the title compound as a white solid (76%); m/z 264.

### Methods 7-11

The following compounds were prepared by the procedure of Method 6 using the appropriate SM and N-bromosuccinimide.

| Meth | Compound | m/z | SM |
|---|---|---|---|
| 7 | Methyl 3-(bromomethyl)-4-fluorobenzoate | 248 | Method 5 |
| 8 | Methyl 3-(bromomethyl)-5-methylbenzoate | 244 | Methyl 3,5-dimethylbenzoate |
| 9 | Methyl 3-(bromomethyl)-1-methyl-1H- | 234 | Methyl 1,3-dimethyl-1H- |
| | pyrazole-5-carboxylate | | pyrazole-5-carboxylate |
| 10 | Methyl 5-(bromomethyl)-1-methyl-1H- | 234 | Methyl 1,5-dimethyl-1H- |
| | pyrazole-3-carboxylate | | pyrazole-3-carboxylate |
| 11 | Methyl 5-(bromomethyl)-2-furoate | 220 | Methyl 5-methyl-2-furoate |

### Method 12

### 3-Cyanomethyl-benzoic acid methyl ester

A suspension of methyl-3-(bromomethyl)benzoate (13.5 g, 58.9 mmol) and sodium cyanide (4.33 g, 88.4 mmol) in DW (25 ml) and water (1 ml) was stirred at 75° C. for 5 hours. The reaction mixture was quenched with water (50 ml) and extracted with EtOAc. The combined organics were dried and concentrated under reduced pressure. The resulting residue was purified by column chromatography utilizing an ISCO system (hexane-EtOAc) to give 7.2 g (70%) of colourless oil. NMR: 7.90 (s, 1H), 7.86 (d, 1H), 7.60 (d, 1H), 7.50 (m, 1H), 4.10 (s, 2H), 3.80 (s, 3H); m/z 175.

### Methods 13-20

The following compounds were prepared by the procedure of Method 12 using the appropriate SM and sodium cyanide.

| Meth | Compound | m/z | SM |
|---|---|---|---|
| 13 | Methyl 4-chloro-3-(cyanomethyl)benzoate | 210 | Method 6 |
| 14 | Methyl 3-(cyanomethyl)-4-fluorobenzoate | 194 | Method 7 |
| 15 | Methyl 3-(cyanomethyl)-5-methylbenzoate | 190 | Method 8 |
| 16 | Methyl 3-(cyanomethyl)-1-methyl-1H- | 180 | Method 9 |
| | pyrazole-5-carboxylate | | |
| 17 | Methyl 5-(cyanomethyl)-1-methyl-1H- | 180 | Method 10 |
| | pyrazole-3-carboxylate | | |
| 18 | Methyl 5-(cyanomethyl)-2-furoate | 166 | Method 11 |
| 19 | [4-({[tert- | 392 | Method 117 |
| | Butyl(diphenyl)silyl]oxy}methyl)-2- | | |
| | thienyl]acetonitrile | | |
| 20 | Methyl 4-(cyanomethyl)thiophene-2- | 182 | Method 118 |
| | carboxylate | | |

### Method 21

### Dimethyl 5-(benzyloxy)isophthalate

To a mixture of dimethyl 5-hydroxyisophthalate (17.3 grams, 82.3 mmol) and potassium carbonate (22.7 grams, 164.6 mmol) in DMF (200 ml) was added benzyl bromide (10.8 ml, 90.5 mmol) and the reaction mixture was allowed to stir at 25° C. for 2 h. The reaction mixture was diluted with EtOAc (750 ml) and washed with water (200 ml). The organic phase was retained and washed with H₂O then brine and dried. The solvent was removed under reduced pressure to give the title compound (25.5 g, 91.1%); NMR (300 MHz): 8.23, (s, 1H), 7.78 (s, 2H), 7.25-7.40 (m, 5H), 3.87 (s, 6H).

### Method 22

### 3-(Benzyloxy)-5-(methoxycarbonyl)benzoic acid

To a solution of the dimethyl 5-(benzyloxy)isophthalate (Method 21; 24.7 g, 82.2 mmol) in 200 ml THF, 200 ml methanol and 50 ml water was added NaOH (2.96 grams, 74.0 mmol) and the reaction mixture was allowed to stir at 25° C. for 15 h. The reaction mixture was concentrated to one-third its original volume under reduced pressure and adjusted to pH 10 with 2 N NaOH. The reaction mixture was washed with EtOAc (75 ml) and the aqueous phase was retained. The aqueous phase was acidified with concentrated HCl to pH 2 and extracted with EtOAc, dried and the solvent was removed under reduced pressure to provide the title compound (12.5 g, 53.2% yield); NMR (300 MHz) 8.29 (s, 1H), 7.82 (s, 2H), 7.26-7.40 (m, 5H), 5.09 (s 2H), 3.88 (s, 3H).

### Method 23

### Methyl 3-(benzyloxy)-5-(hydroxymethyl)benzoate

To a stirring suspension of the 3-(benzyloxy)-5-(methoxycarbonyl)benzoic acid (Method 22; 11.5 g, 40.2 mmol) and triethylamine (13.5 ml, 96.5 mmol) in DCM (200 ml) at 0° C. was added isobutylchloroformate (1.05 ml, 48.2 mmol) over a period of 15 min. The reaction was allowed to warm to 25° C. The reaction mixture was filtered over diatomaceous earth and the solvent was removed under reduced pressure. To the resulting crude mixed anhydride in tetrahydrofuran (200 ml) and water (30 ml) was added sodium borohydride (2.0 g, 52.9 mmol) over 15 min. After stirring at 25° C. for 1 h, additional sodium borohydride (1 g, 26.4 mmol) was added and the reaction was stirred for 1 h. The reaction mixture was concentrated under reduced pressure to one quarter of its original volume then diluted with water (100 ml) and extracted with EtOAc (100 ml). The aqueous phase was extracted with EtOAc (50 ml) and the combined organic extracts were washed with brine (50 ml) then dried and the solvent was removed under reduced pressure. The resulting residue was purified by column chromatography utilizing an ISCO system (hexane-EtOAc) to give 2.9 g of the title compound; NMR (300 MHz): 7.14-7.66 (m, 8H), 5.03 (s, 2H), 4.63 (s, 2H), 3.83 (s, 3H).

### Method 24

### Methyl 3-(hydroxymethyl)-5-nitrobenzoate

Method 24 was prepared following the procedure as described in J. Med. Chem, 2003, Vol. 46, No. 19, 4050-4062; m/z 212.

### Method 25

### Methyl 3-{[(methylsulfonyl)oxy]methyl}-5-nitrobenzoate

To a solution of methyl 3-(hydroxymethyl)-5-nitrobenzoate (Method 24; 790 mg, 3.74 mmol) and triethylamine (680 μl, 4.87 mmol) in DCM was added methanesulfonyl chloride (435 μl, 5.62 mmol) at 0° C. The DCM was removed under reduced pressure and dissolved in EtOAc. The organic layer was washed with 10% HCl aqueous solution, brine, then dried to yield 1.06 g (98%); NMR, 300 MHz) 3.04 (s, 3H), 3.94 (s, 3H), 5.29 (s, 2H), 8.33 (s, 1H), 8.40 (s, 1H), 8.80 (s, 1H).

### Methods 26-27

The following compounds were prepared by the procedure of Method 25 using the appropriate SM and methanesulfonyl chloride.

| Meth | Compound | NMR | SM |
|---|---|---|---|
| 26 | Methyl 3-(benzyloxy)-5- | 7.61 (s, 2H), 7.25-7.88 (m, | Method |
| | {[(methylsulfonyl)oxy] | 6H), 5.15 (s, 2H), 5.05 (s, | 23 |
| | methyl}benzoate | 2H), 3.85 (s, 3H), 2.86 (s 3H) | |
| 27 | Methyl 3-bromo-5- | 8.16 (s, 1H), 7.99 (s, 1H), | Method |
| | {[(methylsulfonyl)oxy]- | 7.74 (s, 1H), 5.22 (s, 2H), | 125 |
| | methyl}benzoate | 3.93 (s, 3H), 3.03 (s, 3H) | |

### Method 28

### Methyl 3-(benzyloxy)-5-(cyanomethyl)benzoate

Methyl 3-(benzyloxy)-5-{[(methylsulfonyl)oxy]methyl}benzoate (Method 26; 2.14 g, 6.1 mmol) in anhydrous DMF (40 ml) was treated with sodium cyanide (0.45 g, 9.2 mmol) and the reaction mixture was allowed to stir at 25° C. for 1.5 h. The reaction was diluted with EtOAc (100 ml) and washed with water. The organic phase was retained and dried and the solvent was removed under reduced pressure. Chromatography (silica: 20% EtOAc/hexane) provided 0.5 grams of the title compound (30% yield); NMR (300 MHz): 7.61-7.63 (m, 2H), 7.26-7.37 (m, 6H), 5.03 (s, 2H), 3.84 (s, 3H), 3.67 (s, 2H).

### Methods 29-30

The following compounds were prepared by the procedure of Method 28 using the appropriate SM and sodium cyanide.

| Meth | Compound | m/z | SM |
|---|---|---|---|
| 29 | Methyl 3-(cyanomethyl)-5- | 221 | Method 25 |
| | nitrobenzoate | | |
| 30 | Methyl 3-bromo-5- | 255 | Method 27 |
| | (cyanomethyl)benzoate | | |

### Method 31

### 3-(1-Cyano-1-methylethyl)benzoic acid methyl ester

A solution of 3-cyanomethyl-benzoic acid methyl ester (Method 12; 7.2 g, 41.1 mmol) in anhydrous DMSO (80 ml) was treated with sodium hydride (60%, 4.9 g, 123.3 mmol). Methyl iodide was then added dropwise at 0° C. The reaction mixture was stirred at 25° C. for 12 h. The reaction mixture was then quenched with water (200 ml) and extracted with EtOAc. The combined organics were dried and concentrated under reduced pressure. The crude product was purified by column chromatography utilizing an ISCO system (hexane-EtOAc) to give 5.5 g (66%) of a colourless oil; NMR: 8.05 (s, 1H), 7.90 (d, 1H), 7.75 (d, 1H), 7.55 (m, 1H), 3.80 (s, 3H), 1.62 (s, 6H); m/z 203.

### Methods 32-45

The following compounds were prepared by the procedure of Method 31 using the appropriate SM and alkyl iodide.

| Meth | Compound | m/z | SM |
|---|---|---|---|
| 32 | Methyl 3-(1-cyanocyclobutyl)benzoate | 216 | Method 12 and 1,3- |
| | | | dibromopropane |
| 33 | Methyl 3-(4-cyanotetrahydro-2H-pyran-4- | 246 | Method 12 and 2-bromoethyl |
| | yl)benzoate | | ether |
| 34 | Methyl 3-(1-cyanocyclopropyl)benzoate | 202 | Method 12 and 1,2- |
| | | | dibromoethane |
| 35 | 2-Methyl-2-(2-thienyl)propanenitrile | 152 | 2-thienylacetonitrile and |
| | | | methyl iodide |
| 36 | Methyl 3-(benzyloxy)-5-(1-cyano-1- | 310 | Method 28 and methyl iodide |
| | methylethyl)benzoate | | |
| 37 | Methyl 3-(1-cyano-1-methylethyl)-5- | 249 | Method 29 and methyl iodide |
| | nitrobenzoate | | |
| 38 | Methyl 4-chloro-3-(1-cyano-1- | 238 | Method 13 and methyl iodide |
| | methylethyl)benzoate | | |
| 39 | Methyl 3-(1-cyano-1-methylethyl)-4- | 222 | Method 14 and methyl iodide |
| | fluorobenzoate | | |
| 40 | Methyl 3-(1-cyano-1-methylethyl)-5- | 218 | Method 15 and methyl iodide |
| | methylbenzoate | | |
| 41 | Methyl 5-(1-cyano-1-methylethyl)-1- | 208 | Method 17 and methyl iodide |
| | methyl-1H-pyrazole-3-carboxylate | | |
| 42 | Methyl 5-(1-cyano-1-methylethyl)-2- | 194 | Method 18 and methyl iodide |
| | furoate | | |
| 43 | 2-[4-({[tert- | 421 | Method 19 and methyl iodide |
| | Butyl(diphenyl)silyl]oxy}methyl)-2- | | |
| | thienyl]-2-methylpropanenitrile | | |
| 44 | Methyl 4-(1-cyano-1- | 210 | Method 20 and methyl iodide |
| | methylethyl)thiophene-2-carboxylate | | |
| 45 | Methyl 3-(1-cyano-1-methylethyl)-1- | 208 | Method 16 and methyl iodide |
| | methyl-1H-pyrazole-5-carboxylate | | |

### Method 46

### 2-(5-Formyl-2-thienyl)-2-methylpropanenitrile

A solution of 2-methyl-2-(2-thienyl)propanenitrile (Method 35; 260 mg, 1.71 mmol) in THF (5.8 ml) was cooled to −78° C. To the cooled reaction was added 1.26 ml of tert-butyl lithium (1.7 M solution in pentanes) dropwise. The resulting bright yellow mixture was allowed to stir for 1 h before anhydrous DMF (0.330 ml, 4.27 mmol) was added. The reaction was stirred for 6 h at −78° C. before being quenched by the addition of 25 ml of saturated aqueous NH₄Cl. The resulting mixture was extracted with EtOAc. The combined organic phase was washed with brine, dried with MgSO₄ (s), and the solvent was removed under reduced pressure giving 271 mg of the title compound (88%) as a colourless oil; m/z 180.

### Method 47

The following compound was prepared by the procedure of Method 46 using the appropriate SM

| Meth | Compound | m/z | SM |
|---|---|---|---|
| 47 | 4-({[tert-Butyl(diphenyl)silyl]oxy}methyl)- | 381 | Method 115 |
| | thiophene-2-carbaldehyde | | |

### Method 48

### 5-(1-Cyano-1-methylethyl)thiophene-2-carboxylic acid

A solution of 2-(5-formyl-2-thienyl)-2-methylpropanenitrile (Method 46; 0.271 g, 1.51 mmol) in 2-methyl-2-propanol (7.5 ml) and 2-methyl-2-butene (4.5 ml) was treated dropwise with NaClO₂ (1.22 g, 13.60 mmol) and NaH₂PO₄ (1.45 g, 10.57 mmol) in H₂O (7 ml). The reaction mixture was stirred for 30 min. at 25° C. then the solvent was removed under reduced pressure. The product was washed with saturated NaHCO₃ (aq) and extracted with EtOAc. The combined organic extracts were washed with brine (50 ml), dried with MgSO₄ (s), and the solvent was removed under reduced pressure giving 0.265 g of the title compound (90%) as a white solid; m/z 196.

### Methods 49-50

The following compound was prepared by the procedure of Method 48 using the appropriate SM.

| Meth | Compound | m/z | SM |
|---|---|---|---|
| 49 | 5-(1-Cyano-1-methylethyl)thiophene-3- | 196 | Method 120 |
| | carboxylic acid | | |
| 50 | 4-({[tert-Butyl(diphenyl)silyl]oxy}methyl)- | 397 | Method 47 |
| | thiophene-2-carboxylic acid | | |

### Method 51

### 2-Methyl-2-(6-methylpyridin-2-yl)propanenitrile

A solution of 2-fluoro-6-methylpyridine (1.00 g, 9.00 mmol) and 2-methylpropanenitrile in anhydrous toluene (30 ml) was treated with potassium hexamethyldisilazide (13.5 mmol) and the reaction was refluxed for 1 h. before being cooled to 25° C. The reaction was then quenched with saturated aqueous NH₄Cl (50 ml) and the mixture was extracted with EtOAc. The combined organic phase was dried with MgSO₄ (s) and the solvent was removed under reduced pressure. The product was purified on silica gel utilizing an ISCO system (hexanes/EtOAc 5:1) giving 0.990 g (70%) of the title compound as a colourless oil; m/z 162.

### Method 52

### 6-(1-Cyano-1-methylethyl)pyridine-2-carboxylic acid

A solution of 2-methyl-2-(6-methylpyridin-2-yl)propanenitrile (Method 51; 0.850 g, 5.30 mmol) in pyridine (50 ml) was treated with selenium dioxide (2.64 g, 23.87 mmol). The reaction was heated to reflux for 72 h. The pyridine was removed under reduced pressure and the resulting residue was washed with EtOAc (200 ml) and H₂O (100 ml). The organic phase was washed with 1 N HCl and then brine. The organic phase was dried with MgSO₄ (s) and the solvent was removed under reduced pressure. The product was purified on silica gel chromatography utilizing an ISCO system (EtOAc/MeOH 10:1) giving 0.313 g (32%) of the title compound as a white solid; m/z 191.

### Method 53

### 3-(1-Cyano-1-methylethyl)-N-(4-fluoro-3-nitrophenyl)benzamide

To a stirring solution of 3-(1-cyano-1-methylethyl)-benzoic acid (Method 73; 200 mg, 1.06 mmol) in DMF (5 ml) were added 4-fluoro-3-nitroaniline (174 mg, 1.06 mmol), HATU (603 mg, 1.59 mmol) and DIEA (0.55 ml, 3.15 mmol) and the reaction mixture was stirred for 10 hours at 25 C. The reaction mixture was partitioned between EtOAc and H₂O. The organics were washed with H₂O, brine and dried (MgSO₄ (s)). The solvent was removed under reduced pressure affording 330 mg (95%) of the title compound; m/z 328.

### Methods 54-56

The following compounds were prepared by the procedure of Method 53 using the appropriate SM and Method 73.

| Meth | Compound | m/z | SM |
|---|---|---|---|
| 54 | N-(4-Bromo-3-nitrophenyl)- | 389 | 4-bromo-3- |
| | 3-(1-cyano-1- | | nitroaniline |
| | methylethyl)benzamide | | |
| 55 | N-(3-Bromo-4-methyl-5-nitrophenyl)- | 403 | 3-bromo-4-methyl- |
| | 3-(1-cyano-1- | | 5-nitroaniline |
| | methylethyl)benzamide | | |
| 56 | 3-(1-Cyano-1-methylethyl)- | 324 | 4-methyl-3- |
| | N-(4-methyl-3- | | nitroaniline |
| | nitrophenyl)benzamide | | |

### Method 57

### N-(3-Amino-4-fluorophenyl)-3-(1-cyano-1-methylethyl)benzamide

To a solution of ammonium chloride (273 mg, 5.05 mmol) in H₂O (5 ml) were added 3-(1-cyano-1-methylethyl)-N-(4-fluoro-3-nitrophenyl)benzamide (Method 53; 330 mg, 1.01 mmol) in methanol (5 ml) and iron powder (283 mg, 5.05 mmol). The solution was stirred at 78° C. for one hour then filtered at 50° C. The filtrate was collected and the solvent was removed under reduced pressure. The residue was taken up in DCM and filtered. The filtrate was collected and the solvent was removed under reduced pressure affording 163 mg of the title compound (54.7%); m/z 298.

### Methods 58-60

The following compounds were prepared by the procedure of Method 57 using the appropriate SM and iron powder.

| Meth | Compound | m/z | SM |
|---|---|---|---|
| 58 | N-(3-Amino-4-bromophenyl)-3-(1-cyano-1- | 359 | Method 54 |
| | methylethyl)benzamide | | |
| 59 | N-(3-Amino-5-bromo-4-methylphenyl)-3-(1- | 372 | Method 55 |
| | cyano-1-methylethyl)benzamide | | |
| 60 | N-(3-Amino-4-methylphenyl)-3-(1-cyano-1- | 294 | Method 56 |
| | methylethyl)benzamide | | |

### Method 61

### 4-Chlorobenzene-1,3-diamine

To a solution of ammonium chloride (1.57 g, 29 mmol) in H₂O (10 ml) were added 2-chloro-5-nitroaniline (1.0 g, 5.8 mmol) and iron powder (1.62 g, 29 mmol). The solution was stirred at 78° C. for one hour then filtered at 50° C. The filtrate was collected and the solvent was removed under reduced pressure. The residue was taken up in DCM and filtered. The filtrate was collected and the solvent was removed under reduced pressure affording 337 mg of the title compound (41%); m/z 143.

### Method 62

The following compound was prepared by the procedure of Method 61 using the appropriate SM and iron powder.

| Meth | Compound | m/z | SM |
|---|---|---|---|
| 62 | 4-Methoxybenzene-1,3-diamine | 139 | 2-methoxy-5-nitroniline |

### Method 63

### N-(3-Amino-4-chlorophenyl)-3-(1-cyano-1-methylethyl)benzamide

To a stirring solution of 3-(1-cyano-1-methylethyl)-benzoic acid (Method 73; 268 mg, 1.41 mmol) in DMF (10 ml) were added 4-chlorobenzene-1,3-diamine (Method 61; 337 mg, 2.36 mmol), HATU (808 mg, 2.13 mmol) and DIEA (0.74 ml, 4.25 mmol) and the reaction mixture was stirred for 10 hours at 25° C. The reaction mixture was partitioned between EtOAc and H₂O. The organics were washed with H₂O, brine and dried (MgSO₄ (s)). The solvent was removed under reduced pressure affording 330 mg (98%) of the title compound; m/z 314.

### Method 64

The following compound was prepared by the procedure of Method 63 using the appropriate SM and Method 73.

| Meth | Compound | m/z | SM |
|---|---|---|---|
| 64 | N-(3-Amino-4-methoxyphenyl)-3-(1-cyano-1- | 310 | Method 62 |
| | methylethyl)benzamide | | |

### Method 65

### N-(3-Amino-1-methylphenyl)-3-(trifluoromethyl)benzamide hydrochloride

N-(4-Methyl-3-nitrophenyl)-3-(trifluoromethyl)benzamide (Method 103; 3.7 g, 11.41 mmol) and 10% palladium on carbon (370 mg) in methanol (20 ml) was shaken under 40 psi H₂ for 3 hours. The reaction mixture was then filtered over diatomaceous earth and the solvent was removed under reduced pressure. The residue was taken up in 30 ml 4 N HCl in dioxane and the solvent was removed under reduced pressure to afford the title compound (3.66 g, 97%); m/z 295.

### Method 66

### Methyl 3-(1-cyano-1-methylethyl)-5-hydroxybenzoate

Methyl 3-(benzyloxy)-5-(1-cyano-1-methylethyl)benzoate (Method 36; 0.200 g, 0.65 mmol) and 10% Pd on carbon (0.020 g) in methanol was shaken under 50 psi for 1 h. The reaction mixture was then filtered over diatomaceous earth and the solvent was removed under reduced pressure; NMR (300 MHz): 7.68 (s, 1H), 7.45 (s, 1H), 7.19 (s, 1H), 3.86 (s, 3H), 1.67 (s, 6H).

### Method 67

### Methyl 3-amino-5-(1-cyano-1-methylethyl)benzoate

Methyl 3-(1-cyano-1-methylethyl)-5-nitrobenzoate (Method 37; 0.068 g, 0.27 mmol) and 10% Pd on carbon (5 mg) in methanol was shaken under 50 psi for 3 h. The reaction mixture was then filtered over diatomaceous earth and the solvent was removed under reduced pressure; m/z 249.

### Method 68

### Methyl 3-(1-cyano-1-methylethyl)-5-(dimethylamino)benzoate

Methyl 3-amino-5-(1-cyano-1-methylethyl)benzoate (Method 67; 290 mg, 1.33 mmol) in MeCN (10 ml) was treated with potassium carbonate (550 mg, 3.99 mmol) and iodomethane (420 μl, 6.65 mmol). The solution was stirred at 80° C. for 15 h. The solvent was removed under reduced pressure and the resulting residue was taken up in EtOAc (100 ml), and washed with water. The organics were dried with NaCl (sat) and then Na₂SO₄ (s) and removed under reduced pressure to give 261 mg (80%) of crude orange oil; m/z 246.

### Method 69

### Methyl 3-[(tert-butoxycarbonyl)amino]-5-(1-cyano-1-methylethyl)benzoate

Di-tert-butyl dicarbonate (69 mg, 0.215 mmol) was added to a stirring solution of methyl 3-amino-5-(1-cyano-1-methylethyl)benzoate (Method 67; 57 mg, 0.261 mmol) and potassium carbonate (108 mg, 0.784 mmol) in THF:H₂O (3:1). The reaction mixture was allowed to stir for 15 h and the water layer was separated. The organic layer was reserved and the solvent was removed under reduced pressure. The product was purified on silica gel utilizing the Isco system (30% EtOAc in hexanes) to give the title compound (41 mg, 50%) as a white solid; m/z 318.

### Method 70

### Methyl 3-[bis(methylsulfonyl)amino]-5-(1-cyano-1-methylethyl)benzoate

To a solution of methyl 3-amino-5-(1-cyano-1-methylethyl)benzoate (Method 67; 350 mg, 1.60 mmol) and DIEA (0.838 ml, 4.8 mmol) in DCM was added methanesulfonyl chloride (0.310 ml, 4.0 mmol). The reaction mixture was allowed to stir for 1 h at 25° C. The solvent was removed under reduced pressure and the residue was taken up in EtOAc and washed with 10% HCl (aq). The organics were dried with NaCl (sat) and then Na₂SO₄ (s) and removed under reduced pressure to give the title compound (430 mg, 72%) as a yellow solid; NMR (300 MHz): 8.26 (s, 1H), 8.01 (s, 1H), 7.69 (s, 1H), 3.98 (s, 3H), 3.46 (s, 6H), 1.81 (s, 6H).

### Method 71

### Methyl 3-(acetylamino)-5-(1-cyano-1-methylethyl)benzoate

To a solution of methyl 3-amino-5-(1-cyano-1-methylethyl)benzoate (Method 67; 300 mg, 1.37 mmol) and triethylamine (0.210 ml, 1.51 mmol) in DCM was added acetyl chloride (0.108 ml, 1.51 mmol). The reaction mixture was allowed to stir for 1 h at 25° C. The solvent was removed under reduced pressure and the residue was taken up in EtOAc and washed with 10% HCl (aq). The organics were dried with NaCl (sat) and then Na₂SO₄ (s) and removed under reduced pressure to give the title compound (218 mg, 92%); nzlz 261.

### Method 72

### 3-Isopropylbenzoic acid

A solution of 1-bromo-3-isopropylbenzene (500 mg, 2.51 mmol) in pentane/ether (1:1; 8 ml) was treated with t-butyllithium (1.7 M in pentane, 3.0 ml) at −78° C. The mixture was stirred at −78° C. for 10 min and then CO₂ (g) was bubbled into the mixture for several minutes. The reaction was quenched with 10% HCl and extracted with EtOAc. The organic layer was dried with NaCl (sat) then Na₂SO₄ (s). The solvents were removed under reduced pressure to give a white solid (379 mg, 92%); m/z 166.

### Method 73

### 3-(1-Cyano-1-methylethyl)benzoic acid

A solution of 3-(1-cyano-1-methylethyl)benzoic acid methyl ester (Method 31; 5.5 g, 27.1 mmol) in 100 ml of THF/MeOH/H₂O (3:1:1) was treated with lithium hydroxide (1.95 g) in 20 ml water. The mixture was stirred at 25° C. for 12 h. The solvent was removed under reduced pressure and the resulting solution was diluted with water, then acidified with 10% HCl to pH=1-3. The resulting white solid (4.83 g, 94%) was filtered, washed with water and dried; NMR: 13.00 (s, 1H), 7.95 (s, 1H), 7.80 (d, 1H), 7.65 (d, 1H), 7.45 (m, 1H), 1.60 (s, 6H); m/z 189.

### Methods 74-102

The following compounds were prepared by the procedure of Method 73 using the appropriate SM and lithium hydroxide.

| Meth | Compound | m/z | SM |
|---|---|---|---|
| 74 | 3-(1-Cyanocyclobutyl)benzoic acid | 202 | Method 32 |
| 75 | 3-(4-Cyanotetrahydro-2H-pyran-4-yl)benzoic acid | 232 | Method 33 |
| 76 | 3-(1-Cyanocyclopropyl)benzoic acid | 188 | Method 34 |
| 77 | 3-(Benzyloxy)-5-(1-cyano-1-methylethyl)benzoic acid | 296 | Method 36 |
| 78 | 4-Chloro-3-(cyanomethyl)benzoic acid | 196 | Method 13 |
| 79 | 4-Chloro-3-(1-cyano-1-methylethyl)benzoic acid | 224 | Method 38 |
| 80 | 3-(1-Cyano-1-methylethyl)-4-fluorobenzoic acid | 208 | Method 39 |
| 81 | 3-(1-Cyano-1-methylethyl)-5-methylbenzoic acid | 204 | Method 40 |
| 82 | 3-(1-Cyano-1-methylethyl)-5-hydroxybenzoic acid | 206 | Method 66 |
| 83 | 3-(1-Cyano-1-methylethyl)-5-(dimethylamino)benzoic | 233 | Method 68 |
| | acid | | |
| 84 | 3-[(tert-Butoxycarbonyl)amino]-5-(1-cyano-1- | 305 | Method 69 |
| | methylethyl) benzoic acid | | |
| 85 | 3-(1-Cyano-1-methylethyl)-5- | 283 | Method 70 |
| | [(methylsulfonyl)amino]benzoic acid | | |
| 86 | 3-(Acetylamino)-5-(1-cyano-1-methylethyl)benzoic acid | 247 | Method 71 |
| 87 | 3-[(1-Hydroxycyclopentyl)ethynyl]benzoic acid | 231 | Method 110 |
| 88 | 3-(3-Cyclopentylprop-1-yn-1-yl)benzoic acid | 229 | Method 108 |
| 89 | 3-(Cyclopropylethynyl)benzoic acid | 187 | Method 109 |
| 90 | 3-[(2-Methoxyethyl)(methyl)amino]benzoic acid | 210 | Method 113 |
| 91 | 5-Piperidin-1-ylnicotinic acid | 207 | Method 112 |
| 92 | 3-Cyclopropylbenzoic acid | 163 | Method 114 |
| 93 | 3-(1-Cyano-1-methylethyl)-1-methyl-1H-pyrazole-5- | 194 | Method 45 |
| | carboxylic acid | | |
| 94 | 5-(1-Cyano-1-methylethyl)-1-methyl-1H-pyrazole-3- | 194 | Method 41 |
| | carboxylic acid | | |
| 95 | 5-(1-Cyano-1-methylethyl)-2-furoic acid | 180 | Method 42 |
| 96 | 4-(1-Cyano-1-methylethyl)thiophene-2-carboxylic acid | 196 | Method 44 |
| 97 | 3-Bromo-5-(methoxycarbonyl)benzoic acid | 259 | Dimethyl 5- |
| | | | bromoisophthalate |
| 98 | 3-(1-Cyano-1-methylethyl)-5-(3-hydroxyprop-1-yn-1- | 243 | Method 111 |
| | yl)benzoic acid | | |
| 99 | 3-Propyl-5-(1-cyano-1-methylethyl)benzoic acid | 245 | Method 126 |
| 100 | 3-(1-Cyano-1-methylethyl)-5-[3-(4-methylpiperazin-1- | 325 | Method 127 |
| | yl)prop-1-yn-1-yl]benzoic acid | | |
| 101 | 3-(Cyano-dimethyl-methyl)-5-(2-pyrrolidin-1-yl- | 302 | Method 129 |
| | ethoxy)benzoic acid | | |
| 102 | 3-(3,3-Dimethylbut-1-yn-1-yl)benzoic acid | 203 | Method 107 |

### Method 103

### N-(4-Methyl-3-nitrophenyl)-3-(trifluoromethyl)benzamide

3-(Trifluoromethyl)benzoyl chloride (2.70 g, 12.95 mmol) in 10 ml anhydrous DCM was added to 4-methyl-3-nitroaniline (1.9 g, 12.95 mmol), and TEA (5.4 ml, 38.85 mmol) in DCM (65 ml) and the reaction mixture was allowed to stir at 25° C. for 1 h. The resulting mixture was washed with 1 N HCl, water and brine. The organic extracts were dried and solvent was removed under reduced pressure to give the title compound as a pale yellow solid (3.70 g, 88%); m/z 325.

Method 104

### Methyl 2-oxo-1,2,3,4-tetrahydroquinoxaline-6-carboxylate

A mixture methyl 3-[(2-methoxy-2-oxoethyl)amino]-4-nitrobenzoate (Method 130; 0.90 g, 3.36 mmol) and 10% Pd on carbon (180 mg) in methanol (30 ml) was shaken under 40 psi H₂ for 30 min. The reaction mixture was then filtered over diatomaceous earth and the solvent was removed under reduced pressure; m/z 207.

### Method 105

### 2-Oxo-1,2-dihydroquinoxaline-6-carboxylic acid

Methyl 2-oxo-1,2,3,4-tetrahydroquinoxaline-6-carboxylate (Method 104; 0.730 g, 3.54 mmol) in 10 ml 1 N NaOH and 10 ml 3% H₂O₂ was stirred at 100° C. for 2 h. The reaction mixture was cooled to 25° C. and acidified to pH 4 with 1 N HCl. The product was collected by vacuum filtration to give 0.450 g (67%); m/z 191.

### Method 106

### 2-Chloroquinoxaline-6-carbonyl chloride

2-Oxo-1,2-dihydroquinoxaline-6-carboxylic acid (Method 105; 0.450 g, 2.37 mmol) in thionyl chloride (5 ml) and DMF (3 drops) was stirred at 90° C. for 3 h. The solvents were removed under reduced pressure and the resultant product was used without further purification; m/z 227.

### Method 107

### Ethyl 3-(3,3-dimethylbut-1-yn-1-yl)benzoate

Ethyl 3-bromobenzoate (0.500 g, 2.18 mmol), 3,3-dimethylbut-1-yne (0.27 g, 3.27 mmol) and triethylamine (1.53 ml, 10.9 mmol) in acetonitrile (8.70 ml) were treated with Pd(PPh₃)₄ (0.25 g, 0.21 mmol) and CuI (0.083 g, 0.436 miol). The reaction was warmed to 60° C. for 4 h. The reaction was then diluted with EtOAc, filtered through a pad of SiO₂, and concentrated in vacuo. The crude reaction product was purified by column chromatography utilizing an ISCO system (hexanes/EtOAc 10:1) giving 0.45 g of the title compound as a colourless oil (91%); m/z 231.

### Methods 108-111

The following compounds were prepared by the procedure of Method 107 using the appropriate starting materials

| Meth | Compound | M/z | SM |
|---|---|---|---|
| 108 | Ethyl 3-(3-cyclopentylprop-1-yn-1- | 256 | Prop-2-yn-1-ylcyclopentane and |
| | yl)benzoate | | ethyl 3-bromobenzoate |
| 109 | Ethyl 3-(cyclopropylethynyl) | 215 | Ethynylcyclopropane and ethyl 3- |
| | benzoate | | bromobenzoate |
| 110 | Ethyl 3-[(1-hydroxycyclopentyl) | 273 | 1-Ethynylcyclopentanol and ethyl 3- |
| | ethynyl]benzoate | | bromobenzoate |
| 111 | Methyl 3-(1-cyano-1-methylethyl)-5- | 258 | Prop-2-yn-1-ol and Method 131 |
| | (3-hydroxyprop-1-yn-1-yl)benzoate | | |

### Method 112

### Methyl 5-piperidin-1-ylnicotinate

Methyl 5-bromonicotinate (0.500 g, 2.31 mmol) and piperidine (0.305 g, 3.46 mmol) in toluene (5 ml) were treated with caesium carbonate (2.25 g, 6.93 mmol), palladium (II) acetate (52 mg, 0.23 mmol), and BINAP (0.287 g, 0.46 mmol). The reaction was heated to 80° C. for 8 h before being diluted with EtOAc, filtered through a pad of SiO₂, and concentrated in vacuo. The crude reaction product was purified by column chromatography utilizing an ISCO system (EtOAc) giving 0.376 g of the title compound as a colourless oil (74%); m/z 221.

### Method 113

The following compound was prepared by the procedure of Method 112 using the appropriate SM and ethyl 3-bromobenzoate

| Meth | Compound | m/z | SM |
|---|---|---|---|
| 113 | Ethyl 3-[(2-methoxyethyl)(methyl)- | 238 | (2-Methoxyethyl)- |
| | amino]benzoate | | methylamine |

### Method 114

### Methyl 3-cyclopropylbenzoate

Diethyl zinc (12.3 ml, 1M in hexanes) in DCM (20 ml) was cooled to 0° C. and then treated with trifluoroacetic acid (1.40 g, 12.3 mmol) by dropwise addition. The reaction was stirred at 0° C. for 20 min and CH₂I₂ (3.30 g, 12.3 mmol) was then added. The reaction mixture was stirred for 20 min before methyl 3-vinylbenzoate (1.00 g, 6.16 mmol) was added. The reaction was then allowed to warm to room temperature with stirring for 3 h. before being quenched by the addition of 50 ml of saturated aqueous NH₄Cl. The mixture was poured into a separatory funnel and the aqueous phase was further extracted with DCM. The combined organic extract was dried with MgSO₄ (s) and concentrated in vacuo to yield the crude reaction product which was purified on 120 g SiO₂ using hexanes/EtOAc 10:1 as eluent giving 1.01 g methyl 3-cyclopropylbenzoate as a colourless oil (94%), m/z 177.

### Method 115

### tert-Butyl(diphenyl)(3-thienylmethoxy)silane

A solution of 3-thienylmethanol (5.0 g, 43.8 mmol) and imidazole (8.94 g, 131.4 mmol) in DMF (86 ml) was treated with tert-butylchlorodiphenylsilane (15.0 g, 54.7 mmol) at 0° C. The reaction stirred for 6 h at 25° C. before being quenched by the addition of 250 ml saturated aqueous NH₄Cl. The resulting mixture was extracted with EtOAc. The combined organic phase was washed once with NaCl (sat) (100 ml), dried with MgSO₄ (s), and concentrated under reduced pressure. The crude reaction product was purified by column chromatography utilizing an ISCO system (hexanes/EtOAc 10:1) giving 14.8 g of the title compound as a colourless oil (96%); m/z 353.

### Method 116

### [4-({[tert-Butyl(diphenyl)silyl]oxy}methyl)-2-thienyl]methanol

4-({[tert-Butyl(diphenyl)silyl]oxy}methyl)thiophene-2-carbaldehyde (Method 47; 3.99 g, 10.48 mmol) was dissolved in MeOH (50 ml). With stirring, NaBH₄ (0.792 g, 20.96 mmol) was added in one portion. After 1 h, the reaction was carefully quenched with a solution of NH₄Cl (sat) (˜250 ml). The resulting mixture was extracted with EtOAc. The combined organic phase was washed with NaCl(sat) (250 ml), dried with MgSO₄ (s), and concentrated in vacuo giving the crude reaction product which was purified on 120 g SiO₂ using hexanes/EtOAc 5:2 as eluent giving 3.99 g of the title compound as a colourless oil (98%); m/z 384.

Method 117

### {[5-(Bromomethyl)-3-thienyl]methoxy}(tert-butyl)diphenylsilane

A solution of [4-({[tert-butyl(diphenyl)silyl]oxy}methyl)-2-thienyl]methanol (Method 116; 4.2 g, 10.98 mmol) in THF (5 ml) was treated with phosphorous tribromide (3.56 g, 13.17 mmol). The reaction was stirred for 1 h. at 25° before being quenched saturated aqueous NaHCO₃ (10 ml). The reaction mixture was extracted with EtOAc and the combined organic phase was dried with MgSO₄ (s) and concentrated under reduced pressure. The product was purified by column chromatography utilizing an ISCO system (hexanes/EtOAc 10:1) giving 3.70 g of the title compound as a yellow oil (76%); m/z 447.

### Method 118

The following compound was prepared by the procedure of Method 117 using the appropriate SM

| Meth | Compound | m/z | SM |
|---|---|---|---|
| 118 | Methyl 4-(bromomethyl)thiophene-2- | 236 | Method 121 |
| | carboxylate | | |

### Method 119

### 2-[4-(Hydroxymethyl)-2-thienyl]-2-methylpropanenitrile

Anhydrous THF (25 ml) was added to 2-[4-({[tert-butyl(diphenyl)silyl]oxy}methyl)-2-thienyl]-2-methylpropanenitrile (Method 43; 0.880 g, 2.10 mmol). A 1 M solution of tetrabutylammonium fluoride in THF (5.25 mmol) was added dropwise via syringe and the reaction was allowed to stir for 12 h at 25° C. before being quenched with NH₄Cl (sat). The reaction mixture was extracted with EtOAc and the combined organic phase was dried with MgSO₄ (s) and concentrated in vacuo. The product was purified by column chromatography utilizing an ISCO system (hexanes/EtOAc 2:1) giving 0.270 g of the title compound as a colourless oil (71%); m/z 182.

### Method 120

### 2-(4-Formyl-2-thienyl)-2-methylpropanenitrile

DMSO (0.277 g, 3.55 mmol) in DCM (10 ml) was cooled to −78° C. and treated with oxalyl chloride (0.225 g, 1.78 mmol). The reaction was allowed to stir for 30 min at −78° C. A 1 M solution of 2-[4-(hydroxymethyl)-2-thienyl]-2-methylpropanenitrile (Method 119; 0.270 g, 1.48 mmol) in DCM was then added dropwise via syringe and the reaction was allowed to stir for 30 min. Triethylamine (0.718 g, 7.40 mmol) was then added and the reaction was allowed to warm to 25° C. with stirring over 1 h before being quenched with NaHCO₃(sat). The reaction mixture was then extracted with EtOAc and the combined organic phase was dried with MgSO₄ (s) and concentrated in vacuo.

### Method 121

### Methyl 4-(hydroxymethyl)thiophene-2-carboxylate

A solution of 4-({[tert-butyl(diphenyl)silyl]oxy}methyl)thiophene-2-carboxylic acid (Method 50; 0.900 g, 2.27 mmol) in MeOH (50 ml) was treated with concentrated HCl (1.0 ml). The reaction was heated at reflux for 12 h and then concentrated under reduced pressure. The crude reaction product was washed with saturated aqueous NaHCO₃ (100 ml) and extracted with EtOAc. The organic phase was dried with MgSO₄ (s) and concentrated under reduced pressure. The product was purified by column chromatography utilizing an ISCO system (hexanes/EtOAc 3:1) giving 0.190 g of the title compound as a colourless oil (50%); m/z 173.

### Method 122

### 2-Methyl-2-(4-methylpyridin-2-yl)propanenitrile

A solution of 2-fluoro-4-methylpyridine (1.00 g, 9.00 mmol) and 2-methylpropanenitrile in toluene (30 ml) was treated with potassium hexamethyldisilazide (13.5 mmol) and the reaction was refluxed for 1 h before being cooled to 25° C. The reaction was then quenched with saturated aqueous NH₄Cl (50 ml) and the mixture was extracted with EtOAc. The combined organic phase was dried with MgSO₄ (s) and concentrated under reduced pressure. The product was purified by column chromatography utilizing an ISCO system (hexanes/EtOAc 5:1) giving 0.990 g of the title compound as a colourless oil (70%); m/z 162.

### Method 123

### 2-(1-Cyano-1-methylethyl)isonicotinic acid

A 50 ml three neck flask equipped with a reflux condenser was charged with a magnetic stir bar, 2-methyl-2-(4-methylpyridin-2-yl)propanenitrile (Method 122; 0.870 g, 5.43 mmol), and water (15 ml). The reaction mixture was heated to 60° C. and KMnO₄ (4.3 g, 27 mmol) was added. The reaction was heated to reflux for 2 h, and was then filtered through a bed of Celite. The pH was adjusted to 4 by the careful addition of 1N HCl and the aqueous phase was extracted with EtOAc. The organic phase was dried with MgSO₄ (s) and concentrated in vacuo to yield the crude reaction product which was purified by column chromatography utilizing an ISCO system (EtOAc/MeOH 10:1) giving 0.700 g of the title compound as a white solid (68%); m/z 191.

### Method 124

The following compound was prepared by the procedure of Method 123 using the appropriate SM

| Meth | Compound | m/z | SM |
|---|---|---|---|
| 124 | 3-tert-Butylbenzoic acid | 179 | 1-ter-Butyl-3-methylbenzene |

### Method 125

### Methyl 3-bromo-5-(hydroxymethyl)benzoate

A solution of 3-bromo-5-(methoxycarbonyl)benzoic acid (Method 97; 1.2 g, 4.6 mmol) in anhydrous THF (20 ml) was treated with BH₃-dimethyl sulfide (2.0 M in THF, 3.5 ml, 6.9 mmol) dropwise under nitrogen at 0° C. The mixture was stirred at 0° C. for 30 min then heated up to 60° C. for 12 hours. The reaction mixture was quenched with H₂O-acetic acid (1:2) (3 ml) and then diluted with EtOAc. The organics were washed with NaHCO₃ (sat) and then dried with NaCl (sat) followed by Na₂SO₄ (s). The solvents were removed under reduced pressure to give the desired product.

### Method 126

### Methyl 3-propyl-5-(1-cyano-1-methylethyl)benzoate

Methyl 3-(1-cyano-1-methylethyl)-5-(3-hydroxyprop-1-yn-1-yl)benzoate (Method 111; 78 mg, 0.30 mmol) in MeOH (3 ml) was treated with Pd/C (10 mg). The reaction mixture was stirred for 12 h under an atmosphere of hydrogen gas at 25° C. The mixture was filtered through celite, and the solvent was removed under reduced pressure to yield the product (26 mg, 34%); NMR (300 MHz): 7.89 (s, 1H), 7.79 (s, 1H), 7.48 (s, 1H), 3.88 (s, 3H), 2.62 (t, 2H), 1.75-1.59 (m, 8H), 0.91 (s, 3H).

### Method 127

### Methyl 3-(1-cyano-1-methylethyl)-5-[3-(4-methylpiperazin-1-yl)prop-1-yn-1-yl]benzoate

A solution of methyl 3-(1-cyano-1-methylethyl)-5-(3-hydroxyprop-1-yn-1-yl)benzoate (Method 111; 115 mg, 0.447 mmol) and triethylamine (81 μl, 0.581 mmol) in DCM was treated with methanesulfonyl chloride (52 μl, 0.671 mmol). The reaction mixture was stirred for 15 min at 25° C. The solvents were removed under reduced pressure and the residue was dissolved in EtOAc and washed with brine and then dried over Na₂SO₄(s). The solvents were removed under reduced pressure to give 149 mg of the desired intermediate. The intermediate was dissolved in DCM (3 ml) and treated with triethylamine (190 μl, 1.34 mmol) and N-methyl piperazine (0.5 ml, 4.5 mmol). The solvents were removed under reduced pressure and the product was purified by column chromatography utilizing an ISCO system (DCM/MeOH 10:1) giving 50 mg of the desired compound (33%); m/z 339.

### Method 128

### 3-[(Dimethylamino)sulfonyl]benzoic acid

A solution of 3-(chlorosulfonyl)benzoic acid (2.60 g, 12 mmol) in DCM (20 ml) was treated with dimethylamine (2.0 M in THF, 20 ml, 40 mmol, 3.3 equiv). After 30 min, the reaction was quenched with 10% HCl and extracted with EtOAc. The organics were washed with NaCl (sat) and then dried with Na₂SO₄ (s). The organics were then removed under reduced pressure to give 1.80 g, 65%; m/z 229.

### Method 129

### 3-(Cyano-dimethyl-methyl)-5-(2-pyrrolidin-1-yl-ethoxy)-benzoic acid methyl ester

A suspension of methyl 3-(1-cyano-1-methylethyl)-5-hydroxybenzoate (Method 66; 200 mg, 0.91 mmol), 1-(2-chloroethyl)pyrrolidine hydrochloride (233 mg, 1.37 mol, 1.5 eq), potassium carbonate (1.26 g, 9.13 mmol, 10 eq) and sodium iodide (137 mg, 0.91 mmol, 1 eq) in 10 ml of acetone was heated at reflux for 12 h. The salt was filtered off and the filtrate was concentrated under reduced pressure. The residue was purified by flash column chromatography (DCM/methanol) to yield 150 mg (57%) of a colorless oil. NMR: 7.65 (s, 1H), 7.40 (s, 1H), 7.30 (s, 1H), 4.15 (t, 2H), 3.90 (s, 3H), 2.90 (m, 2H), 2.62 (m, 4H), 1.65 (m, 10H); m/z 316.

### Method 130

### Methyl 3-[(2-methoxy-2-oxoethyl)amino]-4-nitrobenzoate

Glycine methyl ester hydrochloride (1.82 g, 14.5 mmol) was added to a stirring solution of methyl 3-fluoro-4-nitrobenzoate (0.72 g, 3.62 mmol) and DIEA (3.8 ml, 21.7 mmol) in acetonitrile (20 ml) and the reaction mixture was stirred at 80° C. for 4 h. The solvents were removed under reduced pressure and the product was purified on silica gel to give 0.90 g of the title compound (93%); m/z 269.

### Method 131

### Methyl 3-bromo-5-(1-cyano-1-methylethyl)benzoate

A solution of methyl 3-bromo-5-(cyanomethyl)benzoate (Method 30; 600 mg, 2.36 mmol) in anhydrous DMSO (12 ml) was treated with sodium hydride (60%, 284 mg, 7.09 mmol). Iodomethane (0.882 ml, 14.17 mmol) was then added dropwise at 0° C. The reaction mixture was stirred at 25° C. for 12 h. The reaction mixture was then quenched with water (200 ml) and extracted with EtOAc. The combined organics were dried and concentrated under reduced pressure. The crude product was purified by column chromatography utilizing an ISCO system (hexane-EtOAc) to give 635 mg (95%) of a clear oil; NMR (300 MHz): 7.92 (s, 1H), 7.83 (s, 1H), 7.55 (s, 1H), 3.94 (s, 3H), 1.76 (s, 6H).

1. A compound of formula (I): 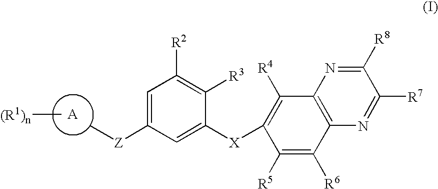
wherein:
Ring A is carbocyclyl or heterocyclyl; wherein if said heterocyclyl contains an —NH— moiety that nitrogen may be optionally substituted by a group selected from R⁹;
R¹ is a substituent on carbon and is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R¹⁰— or heterocyclyl-R¹¹—; wherein R¹ may be optionally substituted on carbon by one or more R¹²; and wherein if said heterocyclyl contains an —NH— moiety that nitrogen may be optionally substituted by a group selected from R¹³;
n is selected from 0-4; wherein the values of R¹ may be the same or different;
Z is —C(O)NH—, —NHC(O)— or —CH₂NH—;
R² is selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁴— or heterocyclyl-R¹⁵—; wherein R² may be optionally substituted on carbon by one or more R¹⁶; and wherein if said heterocyclyl contains an —NH— moiety that nitrogen may be optionally substituted by a group selected from R¹⁷;
R³ is selected from halo, hydroxy, methyl, methoxy or hydroxymethyl;
X is —NR¹⁸C(O)—, —NR¹⁹— or —NR²⁰CH₂—;
R⁴, R⁵, R⁶, R⁷ and R⁸ are independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₄alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₄alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R²¹— or heterocyclyl-R²²—; wherein R⁴, R⁵, R⁶, R⁷ and R⁸ independently of each other may be optionally substituted on carbon by one or more R²³; and wherein if said heterocyclyl contains an —NH— moiety that nitrogen may be optionally substituted by a group selected from R²⁴;
R¹⁸, R¹⁹ and R²⁰ are independently selected from hydrogen, C₁₋₆alkyl, C₁₋₆alkanoyl, C₁₋₆alkylsulphonyl, C₁₋₆alkoxycarbonyl, carbamoyl, N—(C₁₋₆alkyl)carbamoyl and N,N—(C₁₋₆alkyl)carbamoyl; wherein R¹⁸, R¹⁹ and R²⁰ independently of each other may be optionally substituted on carbon by one or more R²⁵;
R¹², R¹⁶, R²³ and R²⁵ are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₄alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R²⁶— or heterocyclyl-R²⁷—; wherein R¹², R¹⁶, R²³ and R²⁵ independently of each other may be optionally substituted on carbon by one or more R²⁸; and wherein if said heterocyclyl contains an —NH— moiety that nitrogen may be optionally substituted by a group selected from R²⁹,
R¹⁰, R¹¹, R¹⁴, R¹⁵, R²¹, R²², R²⁶ and R²⁷ are independently selected from a direct bond, —O—, —N(R³⁰)—, —C(O)—, —N(R³¹)C(O)—, —C(O)N(R³²)—, —S(O)ₛ—, —SO₂N(R³³)— or —N(R³⁴)SO₂—; wherein R³⁰, R³¹, R³², R³³ and R³⁴ is hydrogen or C₁₋₆alkyl and s is 0-2;
R⁹, R¹³, R¹⁷, R²⁴ and R²⁹ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
R²⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
or a pharmaceutically acceptable salt thereof;
with the proviso that said compound is not N-(5-{[3-(dimethylamino)benzoyl]amino}-2-methylphenyl)quinoxaline-6-carboxamide.

2. A compound of formula (I), or a pharmaceutically acceptable salt thereof, according to claim 1 wherein Ring A is phenyl, pyrazolyl, benzimidazolyl, pyridyl, thienyl, furyl, 2,3-dihydro-1,4-benzodioxinyl, 2,3-dihydro-1-benzofuranyl, pyrimidinyl, imidazolyl, indolyl, pyrrolyl or pyrazinyl; wherein said pyrrolyl, pyrazolyl or imidazolyl may be optionally substituted on nitrogen by a group selected from R⁹; wherein R⁹ is selected from C₁₋₆alkyl.

3. A compound of formula (I), or a pharmaceutically acceptable salt thereof, according to either claim 1 or claim 2 wherein R¹ is a substituent on carbon and is selected from halo, nitro, hydroxy, amino, sulphamoyl, C₁₋₆alkyl, C₂₋₆alkynyl, C₁₋₆alkoxy, C₁₋₆alkanoyl, N,N—(C₁₋₆alkyl)₂amino, C₁₋₆alkanoylamino, C₁₋₆alkylS(O)ₐ wherein a is 0 to 2, C₁₋₆alkoxycarbonylamino, N,N—(C₁₋₆alkyl)₂sulphamoyl, C₁₋₆alkylsulphonylamino, carbocyclyl-R¹⁰— or heterocyclyl-R¹¹—; wherein R¹ may be optionally substituted on carbon by one or more R¹²;
R¹² is selected from halo, cyano, hydroxy, C₁₋₆alkyl, C₁₋₆alkoxy, carbocyclyl-R²⁶— or heterocyclyl-R²⁷—; wherein R¹² may be optionally substituted on carbon by one or more R²⁸; and wherein if said heterocyclyl contains an —NH— moiety that nitrogen may be optionally substituted by a group selected from R²⁹;
R¹⁰, R¹¹, R²⁶ and R²⁷ are a direct bond;
R²⁹ is C₁₋₆alkyl;
R²⁸ is selected from hydroxy and methyl.

4. A compound of formula (I), or a pharmaceutically acceptable salt thereof, according to any one of claims 1-3 wherein n is selected from 0-2; wherein the values of R¹ may be the same or different.

5. A compound of formula (I), or a pharmaceutically acceptable salt thereof, according to any one of claims 1-4 wherein Z is —C(O)NH—.

6. A compound of formula (I), or a pharmaceutically acceptable salt thereof, according to any one of claims 1-4 wherein Z is —NHC(O)—.

7. A compound of formula (I), or a pharmaceutically acceptable salt thereof, according to any one of claims 1-4 wherein Z is —CH₂NH—.

8. A compound of formula (I), or a pharmaceutically acceptable salt thereof, according to any one of claims 1-7 wherein R² is selected from hydrogen or halo.

9. A compound of formula (I), or a pharmaceutically acceptable salt thereof, according to any one of claims 1-8 wherein R³ is selected from halo, methyl or methoxy.

10. A compound of formula (I), or a pharmaceutically acceptable salt thereof, according to any one of claims 1-9 wherein X is —NHC(O)—, —NH— or —NHCH₂—.

11. A compound of formula (I), or a pharmaceutically acceptable salt thereof, according to any one of claims 1-10 wherein R⁴, R⁵, R⁶, R⁷ and R⁸ are independently selected from hydrogen, halo, C₁₋₆alkyl, N—(C₁₋₆alkyl)amino, N,N—(C₁₋₆alkyl)₂amino or heterocyclyl-R²²—; wherein R⁴, R⁵, R⁶, R⁷ and R⁸ independently of each other may be optionally substituted on carbon by one or more R²³; wherein
R²³ is selected from hydroxy, amino, N—(C₁₋₆alkyl)amino, N,N—(C₁₋₆alkyl)₂amino or heterocyclyl-R²⁷—; and
R²² and R²⁷ are selected from a direct bond.

12. A compound of formula (I) wherein:
Ring A is phenyl, 1-methylpyrazol-3-yl, 1-methylpyrazol-5-yl, 1-t-butylpyrazol-5-yl, benzimidazol-2-yl, pyrid-2-yl, pyrid-3-yl, pyrid-4-yl, thien-2-yl, thien-3-yl, fur-2-yl, 2,3-dihydro-1,4-benzodioxin-5-yl, 2,3-dihydro-1-benzofuran-7-yl, pyrimidin-4-yl, pyrimidin-5-yl, 1-methylimidazol-2-yl, indol-4-yl, indol-7-yl, 1-methylpyrrol-2-yl or pyrazin-2-yl;
R¹ is a substituent on carbon and is selected from fluoro, chloro, iodo, nitro, hydroxy, amino, sulphamoyl, methyl, trifluoromethyl, cyanomethyl, 3,5-dimethylpyrazol-1-ylmethyl, ethyl, 1-methyl-1-cyanoethyl, propyl, isopropyl, t-butyl, (1-hydroxycyclopentyl)ethynyl, cyclopropylethynyl, 3-hydroxyprop-1-yn-1-yl, 3-(1-methylpiperazin-4-yl)prop-1-yn-1-yl, 3-(cyclopentyl)prop-1-yn-1-yl, 3,3-dimethylprop-1-yn-1-yl, benzyloxy, 2-pyrrolidin-1-ylethoxy, propoxy, isopropoxy, butoxy, isobutoxy, methylthio, difluoromethylthio, mesyl, dimethylamino, N-methyl-N-(2-methoxyethyl)amino, acetyl, N,N-dimethylsulphamoyl, acetylamino, t-butoxycarbonylamino, 2,2-dimethylpropionylamino, mesylamino, cyclopropyl, 1-cyanocyclopropyl, 1-cyanocyclobutyl, 1-cyano-tetrahydro-2H-pyran-4-yl, thien-2-yl, pyrrol-1-yl, 2,5-dimethylpyrrol-1-yl, pyrid-3-yl, 2-methylthiazol-4-yl, morpholino and piperidin-1-yl;
n is selected from 0-2; wherein the values of R¹ may be the same or different;
Z is —C(O)NH—, —NHC(O)— or —CH₂NH—;
R¹ is selected from hydrogen or bromo;
R³ is selected from fluoro, chloro, bromo, methyl or methoxy;
X is —NHC(O)—, —NH— or —NHCH₂—;
R⁴, R⁵, R⁶, R⁷ and R⁸ are independently selected from hydrogen, chloro, methyl, 3-(piperidin-1-yl)propylamino, 2-hydroxyethylamino, 2-(dimethylamino)ethylamino, 2-(morpholino)ethylamino, methylamino, N-methyl-N-ethylamino, N-methyl-N-(2-methylaminoethyl)amino, morpholino, 3-aminopropylamino or N-methyl-N-(3-direthylaminopropyl)amino;
or a pharmaceutically acceptable salt thereof; with the proviso that said compound is not N-(5-{[3-(dimethylamino)benzoyl]amino}-2-methylphenyl)quinoxaline-6-carboxamide.

13. A compound of formula (I) selected from:
N-(5-{[3-(1-cyano-1-methylethyl)-5-(3-hydroxyprop-1-yn-1-yl)benzoyl]amino}-2-methylphenyl)quinoxaline-6-carboxamide;
N-(5-{[3-(1-cyano-1-methylethyl)benzoyl]amino}-2-methylphenyl)quinoxaline-6-carboxamide;
N-[5-({3-(1-cyano-1-methylethyl)-5-[3-(4-methylpiperazin-1-yl)prop-1-yn-1-yl]benzoyl}amino)-2-methylphenyl]quinoxaline-6-carboxamide;
N-(5-{[3-(benzyloxy)-5-(1-cyano-1-methylethyl)benzoyl]amino}-2-methylphenyl) quinoxaline-6-carboxamide;
N-{5-[(3-tert-butylbenzoyl)amino]-2-methylphenyl}quinoxaline-6-carboxamide;
N-(5-{[3-(1-cyano-1-methylethyl)-5-propylbenzoyl]amino}-2-methylphenyl)quinoxaline-6-carboxamide;
N-(5-{[3-(1-cyano-1-methylethyl)-5-(2-pyrrolidin-1-ylethoxy)benzoyl]amino}-2-methylphenyl)quinoxaline-6-carboxamide;
N-(5-{[3-(1-cyano-1-methylethyl)-5-methylbenzoyl]amino}-2-methylphenyl)quinoxaline-6-carboxamide;
N-{5-[(3,5-di-tert-butylbenzoyl)amino]-2-methylphenyl}quinoxaline-6-carboxamide; and
N-(5-{[3-(1-cyanocyclobutyl)benzoyl]amino}-2-methylphenyl)quinoxaline-6-carboxamide;
or a pharmaceutically acceptable salt thereof.

14. A process for preparing a compound of formula (I) or a pharmaceutically acceptable salt thereof which process, wherein variable are, unless otherwise specified, as defined in claim 1, comprises of:
Process a) for compounds of formula (I) wherein Z is —C(O)NH—; reacting an amine of the formula (II) 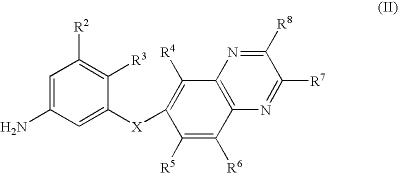
with an acid of formula (III): 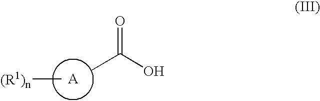
or an activated acid derivative thereof;
Process b) for compounds of formula (I) wherein X is —NR¹⁸C(O)— and R¹⁸ is hydrogen or C₁₋₆alkyl; reacting an amine of formula (IV): 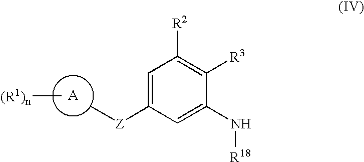
with an acid of formula (V): 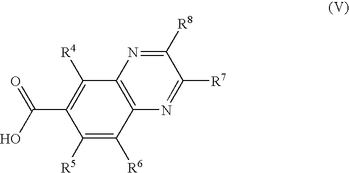
or an activated acid derivative thereof;
Process c) for compounds of formula (I) wherein Z is —CH₂NH—; reacting an amine of the formula (II) with a compound of formula (VI): 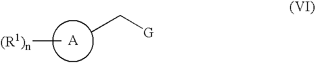
wherein G is a displaceable group;
Process d) for compounds of formula (I) wherein Z is —CH₂NH—; reacting an amine of the formula (VII): 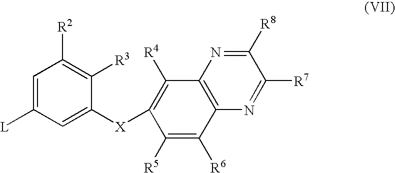
wherein L is a displaceable group; with a compound of formula (VIII): 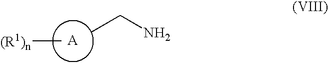
Process e) for compounds of formula (I) wherein X is —NR¹⁹— and R¹⁹ is hydrogen or C₁₋₆alkyl; reacting an amine of formula (IX): 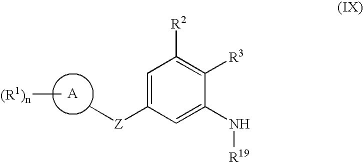
with a compound of formula (X): 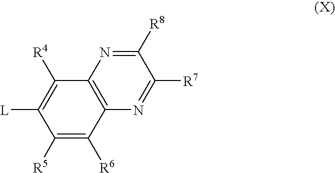
wherein L is a displaceable group
Process f) for compounds of formula (I) wherein X is —NR¹⁹— and R¹⁹ is hydrogen or C₁₋₆alkyl; reacting an amine of formula (IX): 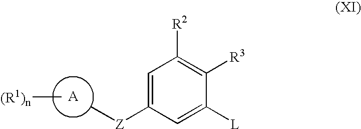
wherein L is a displaceable group; with a compound of formula (XII): 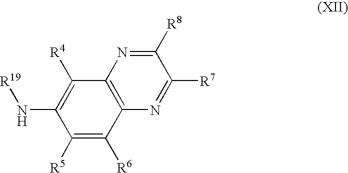
Process g) for compounds of formula (I) wherein X is —NR²⁰CH₂—R²⁰ is hydrogen or C₁₋₆alkyl; reacting an amine of formula (XIII): 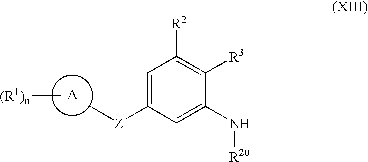
with a compound of formula (XIV): 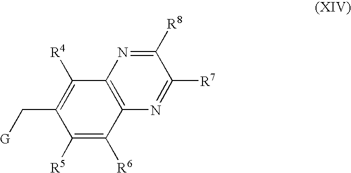
wherein G is a displaceable group;
Process h) for compounds of formula (I) wherein X is —NR²⁰CH₂— wherein R²⁰ is hydrogen or C₁₋₆alkyl; reacting an amine of formula (XIII) with a compound of formula (XVI): 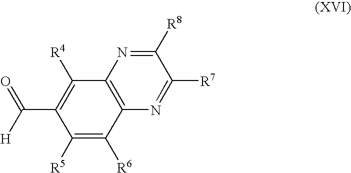
wherein L is a displaceable group
Process i) for compounds of formula (I) wherein Y is —CH₂—; reacting an amine of the formula (II) with a compound of formula (XVII): 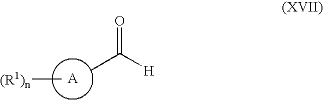
Process j) for compounds of formula (I) where Z is —NHC(O)— reacting a compound of formula (XVIII): 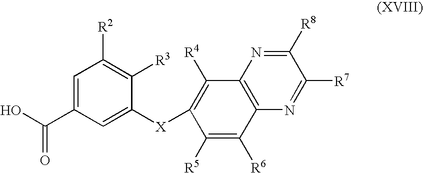
or an activated derivative thereof; with a compound of formula (XIX): 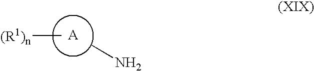
and thereafter if necessary:
i) converting a compound of the formula (I) into another compound of the formula (I);
ii) removing any protecting groups;
iii) forming a pharmaceutically acceptable salt.

15. A pharmaceutical composition which comprises a compound of the formula (I), or a pharmaceutically acceptable salt thereof, as claimed in any one of claims 1-13, in association with a pharmaceutically-acceptable diluent or carrier.

16. A compound of the formula (I), or a pharmaceutically acceptable salt thereof, as claimed in any one of claims 1-13, for use as a medicament.

17. The use of a compound of the formula (I), or a pharmaceutically acceptable salt thereof, as claimed in any one of claims 1-13, in the manufacture of a medicament for use in the production of a B-Raf inhibitory effect in a warm-blooded animal such as man.

18. The use of a compound of the formula (I), or a pharmaceutically acceptable salt thereof, as claimed in any one of claims 1-13, in the manufacture of a medicament for use in the production of an anti-cancer effect in a warm-blooded animal such as man.

19. The use of a compound of the formula (I), or a pharmaceutically acceptable salt thereof, as claimed in any one of claims 1-13, in the manufacture of a medicament for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

20. A method for producing a B-Raf inhibitory effect in a warm-blooded animal, such as man, in need of such treatment which comprises administering to said animal an effective amount of a compound of formula (I), or a pharmaceutically acceptable salt thereof, as claimed in any one of claims 1-13.

21. A method for producing an anti-cancer effect in a warm-blooded animal, such as man, in need of such treatment which comprises administering to said animal an effective amount of a compound of formula (I), or a pharmaceutically acceptable salt thereof, as claimed in any one of claims 1-13.

22. A method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof, as claimed in any one of claims 1-13.

23. A pharmaceutical composition which comprises a compound of the formula (I), or a pharmaceutically acceptable salt thereof, as claimed in any one of claims 1-13, in association with a pharmaceutically-acceptable diluent or carrier for use in the production of a B-Raf inhibitory effect in a warm-blooded animal such as man.

24. A pharmaceutical composition which comprises a compound of the formula (I), or a pharmaceutically acceptable salt thereof, as claimed in any one of claims 1-13, in association with a pharmaceutically-acceptable diluent or carrier for use in the production of an anti-cancer effect in a warm-blooded animal such as man.

25. A pharmaceutical composition which comprises a compound of the formula (I), or a pharmaceutically acceptable salt thereof, as claimed in any one of claims 1-13, in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries in a warm-blooded animal such as man.
